SEQUENCE LISTING (57 sequences)

SEQ ID NO: 1 - DNA, 2433 bp, Homo sapiens
  LRRC8A coding sequence
atgattccggtgacagagctccgctactttgcggacacgcagccagcataccggatcctgaagccgtggtgggatgtgttcacagactacatctctatcgtcatgctgatgattgccgtcttcggggggacgctgcaggtcacccaagacaagatgatctgcctgccttgtaagtgggtcaccaaggactcctgcaatgattcgttccggggctgggcagcccctggcccggagcccacctaccccaactccaccattctgccgacccctgacacgggccccacaggcatcaagtatgacctggaccggcaccagtacaactacgtggacgctgtgtgctatgagaaccgactgcactggtttgccaagtacttcccctacctggtgcttctgcacacgctcatcttcctggcctgcagcaacttctggttcaaattcccgcgcaccagctcgaagctggagcactttgtgtctatcctgctgaagtgcttcgactcgccctggaccacgagggccctgtcggagacagtggtggaggagagcgaccccaagccggccttcagcaagatgaatgggtccatggacaaaaagtcatcgaccgtcagtgaggacgtggaggccaccgtgcccatgctgcagcggaccaagtcacggatcgagcagggtatcgtggaccgctcagagacgggcgtgctggacaagaaggagggggagcaagccaaggcgctgtttgagaaggtgaagaagttccggacccatgtggaggagggggacattgtgtaccgcctctacatgcggcagaccatcatcaaggtgatcaagttcatcctcatcatctgctacaccgtctactacgtgcacaacatcaagttcgacgtggactgcaccgtggacattgagagcctgacgggctaccgcacctaccgctgtgcccaccccctggccacactcttcaagatcctggcgtccttctacatcagcctagtcatcttctacggcctcatctgcatgtatacactgtggtggatgctacggcgctccctcaagaagtactcgtttgagtcgatccgtgaggagagcagctacagcgacatccccgacgtcaagaacgacttcgccttcatgctgcacctcattgaccaatacgacccgctctactccaagcgcttcgccgtcttcctgtcggaggtgagtgagaacaagctgcggcagctgaacctcaacaacgagtggacgctggacaagctccggcagcggctcaccaagaacgcgcaggacaagctggagctgcacctgttcatgctcagtggcatccctgacactgtgtttgacctggtggagctggaggtcctcaagctggagctgatccccgacgtgaccatcccgcccagcattgcccagctcacgggcctcaaggagctgtggctctaccacacagcggccaagattgaagcgcccgcgctggccttcctgcgtgagaacctgcgggcgctgcacatcaagttcaccgacatcaaggagatcccgctgtggatctatagcctgaagacactggaggagctgcacctgacgggcaacctgagcgcggagaacaaccgctacatcgtcatcgacgggctgcgggagctcaaacgcctcaaggtgctgcggctcaagagcaacctaagcaagctgccacaggtggtcacagatgtgggcgtgcacctgcagaagctgtccatcaacaatgagggcaccaagctcatcgtcctcaacagcctcaagaagatggcgaacctgactgagctggagctgatccgctgtgacctggagcgcatcccccactccatcttcagcctccacaacctgcaggagattgacctcaaggacaacaacctcaagaccatcgaggagatcatcagcttccagcacctgcaccgcctcacctgccttaagctgtggtacaaccacatcgcctacatccccatccagatcggcaacctcaccaacctggagcgcctctacctgaaccgcaacaagatcgagaagatccccacccagctcttctactgccgcaagctgcgctacctggacctcagccacaacaacctgaccttcctccctgccgacatcggcctcctgcagaacctccagaacctagccatcacggccaaccggatcgagacgctccctccggagctcttccagtgccggaagctgcgggccctgcacctgggcaacaacgtgctgcagtcactgccctccagggtgggcgagctgaccaacctgacgcagatcgagctgcggggcaaccggctggagtgcctgcctgtggagctgggcgagtgcccactgctcaagcgcagcggcttggtggtggaggaggacctgttcaacacactgccacccgaggtgaaggagcggctgtggagggctgacaaggagcaggcctga

SEQ ID NO: 2 - protein, 810 aa, Homo sapiens
  LRRC8A protein
MIPVTELRYFADTQPAYRILKPWWDVFTDYISIVMLMIAVFGGTLQVTQDKMICLPCKWVTKDSCNDSFRGWAAPGPEPTYPNSTILPTPDTGPTGIKYDLDRHQYNYVDAVCYENRLHWFAKYFPYLVLLHTLIFLACSNFWFKFPRTSSKLEHFVSILLKCFDSPWTTRALSETVVEESDPKPAFSKMNGSMDKKSSTVSEDVEATVPMLQRTKSRIEQGIVDRSETGVLDKKEGEQAKALFEKVKKFRTHVEEGDIVYRLYMRQTIIKVIKFILIICYTVYYVHNIKFDVDCTVDIESLTGYRTYRCAHPLATLFKILASFYISLVIFYGLICMYTLWWMLRRSLKKYSFESIREESSYSDIPDVKNDFAFMLHLIDQYDPLYSKRFAVFLSEVSENKLRQLNLNNEWTLDKLRQRLTKNAQDKLELHLFMLSGIPDTVFDLVELEVLKLELIPDVTIPPSIAQLTGLKELWLYHTAAKIEAPALAFLRENLRALHIKFTDIKEIPLWIYSLKTLEELHLTGNLSAENNRYIVIDGLRELKRLKVLRLKSNLSKLPQVVTDVGVHLQKLSINNEGTKLIVLNSLKKMANLTELELIRCDLERIPHSIFSLHNLQEIDLKDNNLKTIEEIISFQHLHRLTCLKLWYNHIAYIPIQIGNLTNLERLYLNRNKIEKIPTQLFYCRKLRYLDLSHNNLTFLPADIGLLQNLQNLAITANRIETLPPELFQCRKLRALHLGNNVLQSLPSRVGELTNLTQIELRGNRLECLPVELGECPLLKRSGLVVEEDLFNTLPPEVKERLWRADKEQA

SEQ ID NO: 3 - RNA, 4368 nt, Homo sapiens
  LRRC8A mRNA transcript variant 2 (NM_019594)
acttccggggcggaggaggctgagtggtgcagtgagggacaaacaaaaggaggcgccggagcagcgctgcggccggcggcgggacggagcggccggggcctggggctgcctgccgggcggccgggcgcggcgagcccagggaggcagcgtccatggagcaaaaggaatgccaggatcctgcacaggcagacgcgggccagcctcagcaccgacagccgacgcgcagatagcagagccatccttggggttgaaccatgattccggtgacagagctccgctactttgcggacacgcagccagcataccggatcctgaagccgtggtgggatgtgttcacagactacatctctatcgtcatgctgatgattgccgtcttcggggggacgctgcaggtcacccaagacaagatgatctgcctgccttgtaagtgggtcaccaaggactcctgcaatgattcgttccggggctgggcagcccctggcccggagcccacctaccccaactccaccattctgccgacccctgacacgggccccacaggcatcaagtatgacctggaccggcaccagtacaactacgtggacgctgtgtgctatgagaaccgactgcactggtttgccaagtacttcccctacctggtgcttctgcacacgctcatcttcctggcctgcagcaacttctggttcaaattcccgcgcaccagctcgaagctggagcactttgtgtctatcctgctgaagtgcttcgactcgccctggaccacgagggccctgtcggagacagtggtggaggagagcgaccccaagccggccttcagcaagatgaatgggtccatggacaaaaagtcatcgaccgtcagtgaggacgtggaggccaccgtgcccatgctgcagcggaccaagtcacggatcgagcagggtatcgtggaccgctcagagacgggcgtgctggacaagaaggagggggagcaagccaaggcgctgtttgagaaggtgaagaagttccggacccatgtggaggagggggacattgtgtaccgcctctacatgcggcagaccatcatcaaggtgatcaagttcatcctcatcatctgctacaccgtctactacgtgcacaacatcaagttcgacgtggactgcaccgtggacattgagagcctgacgggctaccgcacctaccgctgtgcccaccccctggccacactcttcaagatcctggcgtccttctacatcagcctagtcatcttctacggcctcatctgcatgtatacactgtggtggatgctacggcgctccctcaagaagtactcgtttgagtcgatccgtgaggagagcagctacagcgacatccccgacgtcaagaacgacttcgccttcatgctgcacctcattgaccaatacgacccgctctactccaagcgcttcgccgtcttcctgtcggaggtgagtgagaacaagctgcggcagctgaacctcaacaacgagtggacgctggacaagctccggcagcggctcaccaagaacgcgcaggacaagctggagctgcacctgttcatgctcagtggcatccctgacactgtgtttgacctggtggagctggaggtcctcaagctggagctgatccccgacgtgaccatcccgcccagcattgcccagctcacgggcctcaaggagctgtggctctaccacacagcggccaagattgaagcgcccgcgctggccttcctgcgcgagaacctgcgggcgctgcacatcaagttcaccgacatcaaggagatcccgctgtggatctatagcctgaagacactggaggagctgcacctgacgggcaacctgagcgcggagaacaaccgctacatcgtcatcgacgggctgcgggagctcaaacgcctcaaggtgctgcggctcaagagcaacctaagcaagctgccacaggtggtcacagatgtgggcgtgcacctgcagaagctgtccatcaacaatgagggcaccaagctcatcgtcctcaacagcctcaagaagatggcgaacctgactgagctggagctgatccgctgtgacctggagcgcatcccccactccatcttcagcctccacaacctgcaggagattgacctcaaggacaacaacctcaagaccatcgaggagatcatcagcttccagcacctgcaccgcctcacctgccttaagctgtggtacaaccacatcgcctacatccccatccagatcggcaacctcaccaacctggagcgcctctacctgaaccgcaacaagatcgagaagatccccacccagctcttctactgccgcaagctgcgctacctggacctcagccacaacaacctgaccttcctccctgccgacatcggcctcctgcagaacctccagaacctagccatcacggccaaccggatcgagacgctccctccggagctcttccagtgccggaagctgcgggccctgcacctgggcaacaacgtgctgcagtcactgccctccagggtgggcgagctgaccaacctgacgcagatcgagctgcggggcaaccggctggagtgcctgcctgtggagctgggcgagtgcccactgctcaagcgcagcggcttggtggtggaggaggacctgttcaacacactgccacccgaggtgaaggagcggctgtggagggctgacaaggagcaggcctgagcgaggccggcccagcacagcaagcagcaggaccgctgcccagtcctcaggcccggaggggcaggcctagcttctcccagaactcccggacagccaggacagcctcgtggctgggcaggagcctggggccgcttgtgagtcaggccagagcgagaggacagtatctgtggggctggccccttttctccctctgagactcacgtcccccagggcaagtgcttgtggaggagagcaagtctcaagagcgcagtatttggataatcagggtctcctccctggaggccagctctgccccaggggctgagctgccaccagaggtcctgggaccctcactttagttcttggtatttatttttctccatctcccacctccttcatccagataacttatacattcccaagaaagttcagcccagatggaaggtgttcagggaaaggtgggctgccttttccccttgtccttatttagcgatgccgccgggcatttaacacccacctggacttcagcagagtggtccggggcgaaccagccatgggacggtcacccagcagtgccgggctgggctctgcggtgcggtccacgggagagcaggcctccagctggaaaggccaggcctggagcttgcctcttcagtatttgtggcagttttagttttttgtttttttttttttaatcaaaaaacaatttttttaaaaaaaaagctttgaaaatggatggtttgggtattaaaaagaaaaaaaaaacttaaaaaaaaaaagacactaacggccagtgagttggagtctcagggcagggtggcagtttcccttgagcaaagcagccagacgttgaactgtgtttcctttccctgggcgcagggtgcagggtgtcttccggatctggtgtgaccttggtccaggagttctatttgttcctggggagggaggtttttttgtttgttttttgggtttttttggtgtcttgttttctttctcctccatgtgtcttggcaggcactcatttctgtggctgtcggccagagggaatgttctggagctgccaaggagggaggagactcgggttggctaatccccggatgaacggtgctccattcgcacctcccctcctcgtgcctgccctgcctctccacgcacagtgttaaggagccaagaggagccacttcgcccagactttgtttccccaccgcctgcggcatgggtgtgtccagtgccaccgctggcctccgctgcttccatcagccctgtcgccacctggtccttcatgaagagcagacacttagaggctggtcgggaatggggaggtcgcccctgggagggcaggcgttggttccaagccggttcccgtccctggcgcctggagtgcacacagcccagtcggcacctggtggctggaagccaccctgctttagatcactcgggtccccaccttagaagggtccccgccttagatcaatcacgtggacactaaggcacgttttagagtctcttgtcttaatgattatgtccatccgtctgtccgtccatttgtgttttctgcgtcgtgtcattggatataatcctcagaaataatgcacactagcctctgacaaccatgaagcaaaaatccgttacatgtgggtctgaacttgtagactcggtcacagtatcaaataaaatctataacagaaaaaaaaaaaaaaaa

SEQ ID NO: 4 - RNA, 4637 nt, Homo sapiens
  LRRC8A mRNA transcript variant 1 (NM_001127244)
gtgcttcagaaaccaggagtttccgcctcggctcccccatgtccccttgtcatcccctgggtccccccagatcctcacccctccacacacactctcccgttcctagaggttccacctctgggctctctccttgccatttctttttcaaagatttccttagctgtcttctcctgagttcctggtccccttctcttcccttgcacccctccttcctatcgttccgatgggggcagtgccctgacttgggggcaggatccccggctaggctcttggggcctttctgggatgggatatttgggaagaccggtccggaatctaagaacccagaccctgtcccagtcctgtgcattcaggtgggcccgagggcgaggcgagatccagtgaggtccaggcctggtgcagccctagggaggcagcgtccatggagcaaaaggaatgccaggatcctgcacaggcagacgcgggccagcctcagcaccgacagccgacgcgcagatagcagagccatccttggggttgaaccatgattccggtgacagagctccgctactttgcggacacgcagccagcataccggatcctgaagccgtggtgggatgtgttcacagactacatctctatcgtcatgctgatgattgccgtcttcggggggacgctgcaggtcacccaagacaagatgatctgcctgccttgtaagtgggtcaccaaggactcctgcaatgattcgttccggggctgggcagcccctggcccggagcccacctaccccaactccaccattctgccgacccctgacacgggccccacaggcatcaagtatgacctggaccggcaccagtacaactacgtggacgctgtgtgctatgagaaccgactgcactggtttgccaagtacttcccctacctggtgcttctgcacacgctcatcttcctggcctgcagcaacttctggttcaaattcccgcgcaccagctcgaagctggagcactttgtgtctatcctgctgaagtgcttcgactcgccctggaccacgagggccctgtcggagacagtggtggaggagagcgaccccaagccggccttcagcaagatgaatgggtccatggacaaaaagtcatcgaccgtcagtgaggacgtggaggccaccgtgcccatgctgcagcggaccaagtcacggatcgagcagggtatcgtggaccgctcagagacgggcgtgctggacaagaaggagggggagcaagccaaggcgctgtttgagaaggtgaagaagttccggacccatgtggaggagggggacattgtgtaccgcctctacatgcggcagaccatcatcaaggtgatcaagttcatcctcatcatctgctacaccgtctactacgtgcacaacatcaagttcgacgtggactgcaccgtggacattgagagcctgacgggctaccgcacctaccgctgtgcccaccccctggccacactcttcaagatcctggcgtccttctacatcagcctagtcatcttctacggcctcatctgcatgtatacactgtggtggatgctacggcgctccctcaagaagtactcgtttgagtcgatccgtgaggagagcagctacagcgacatccccgacgtcaagaacgacttcgccttcatgctgcacctcattgaccaatacgacccgctctactccaagcgcttcgccgtcttcctgtcggaggtgagtgagaacaagctgcggcagctgaacctcaacaacgagtggacgctggacaagctccggcagcggctcaccaagaacgcgcaggacaagctggagctgcacctgttcatgctcagtggcatccctgacactgtgtttgacctggtggagctggaggtcctcaagctggagctgatccccgacgtgaccatcccgcccagcattgcccagctcacgggcctcaaggagctgtggctctaccacacagcggccaagattgaagcgcccgcgctggccttcctgcgcgagaacctgcgggcgctgcacatcaagttcaccgacatcaaggagatcccgctgtggatctatagcctgaagacactggaggagctgcacctgacgggcaacctgagcgcggagaacaaccgctacatcgtcatcgacgggctgcgggagctcaaacgcctcaaggtgctgcggctcaagagcaacctaagcaagctgccacaggtggtcacagatgtgggcgtgcacctgcagaagctgtccatcaacaatgagggcaccaagctcatcgtcctcaacagcctcaagaagatggcgaacctgactgagctggagctgatccgctgtgacctggagcgcatcccccactccatcttcagcctccacaacctgcaggagattgacctcaaggacaacaacctcaagaccatcgaggagatcatcagcttccagcacctgcaccgcctcacctgccttaagctgtggtacaaccacatcgcctacatccccatccagatcggcaacctcaccaacctggagcgcctctacctgaaccgcaacaagatcgagaagatccccacccagctcttctactgccgcaagctgcgctacctggacctcagccacaacaacctgaccttcctccctgccgacatcggcctcctgcagaacctccagaacctagccatcacggccaaccggatcgagacgctccctccggagctcttccagtgccggaagctgcgggccctgcacctgggcaacaacgtgctgcagtcactgccctccagggtgggcgagctgaccaacctgacgcagatcgagctgcggggcaaccggctggagtgcctgcctgtggagctgggcgagtgcccactgctcaagcgcagcggcttggtggtggaggaggacctgttcaacacactgccacccgaggtgaaggagcggctgtggagggctgacaaggagcaggcctgagcgaggccggcccagcacagcaagcagcaggaccgctgcccagtcctcaggcccggaggggcaggcctagcttctcccagaactcccggacagccaggacagcctcgtggctgggcaggagcctggggccgcttgtgagtcaggccagagcgagaggacagtatctgtggggctggccccttttctccctctgagactcacgtcccccagggcaagtgcttgtggaggagagcaagtctcaagagcgcagtatttggataatcagggtctcctccctggaggccagctctgccccaggggctgagctgccaccagaggtcctgggaccctcactttagttcttggtatttatttttctccatctcccacctccttcatccagataacttatacattcccaagaaagttcagcccagatggaaggtgttcagggaaaggtgggctgccttttccccttgtccttatttagcgatgccgccgggcatttaacacccacctggacttcagcagagtggtccggggcgaaccagccatgggacggtcacccagcagtgccgggctgggctctgcggtgcggtccacgggagagcaggcctccagctggaaaggccaggcctggagcttgcctcttcagtatttgtggcagttttagttttttgtttttttttttttaatcaaaaaacaatttttttaaaaaaaaagctttgaaaatggatggtttgggtattaaaaagaaaaaaaaaacttaaaaaaaaaaagacactaacggccagtgagttggagtctcagggcagggtggcagtttcccttgagcaaagcagccagacgttgaactgtgtttcctttccctgggcgcagggtgcagggtgtcttccggatctggtgtgaccttggtccaggagttctatttgttcctggggagggaggtttttttgtttgttttttgggtttttttggtgtcttgttttctttctcctccatgtgtcttggcaggcactcatttctgtggctgtcggccagagggaatgttctggagctgccaaggagggaggagactcgggttggctaatccccggatgaacggtgctccattcgcacctcccctcctcgtgcctgccctgcctctccacgcacagtgttaaggagccaagaggagccacttcgcccagactttgtttccccaccgcctgcggcatgggtgtgtccagtgccaccgctggcctccgctgcttccatcagccctgtcgccacctggtccttcatgaagagcagacacttagaggctggtcgggaatggggaggtcgcccctgggagggcaggcgttggttccaagccggttcccgtccctggcgcctggagtgcacacagcccagtcggcacctggtggctggaagccaccctgctttagatcactcgggtccccaccttagaagggtccccgccttagatcaatcacgtggacactaaggcacgttttagagtctcttgtcttaatgattatgtccatccgtctgtccgtccatttgtgttttctgcgtcgtgtcattggatataatcctcagaaataatgcacactagcctctgacaaccatgaagcaaaaatccgttacatgtgggtctgaacttgtagactcggtcacagtatcaaataaaatctataacagaaaaaaaaaaaaaaaa

SEQ ID NO: 5 - RNA, 4261 nt, Homo sapiens
  LRRC8A mRNA transcript variant 3 (NM_001127245)
acttccggggcggaggaggctgagtggtgcagtgagggacaaacaaaaggaggcgccggagcagcgctgcggccggcggcgggacggagcggccggggcctggggctgcctgccgggcggccgggcgcggcgagcccaggttgaaccatgattccggtgacagagctccgctactttgcggacacgcagccagcataccggatcctgaagccgtggtgggatgtgttcacagactacatctctatcgtcatgctgatgattgccgtcttcggggggacgctgcaggtcacccaagacaagatgatctgcctgccttgtaagtgggtcaccaaggactcctgcaatgattcgttccggggctgggcagcccctggcccggagcccacctaccccaactccaccattctgccgacccctgacacgggccccacaggcatcaagtatgacctggaccggcaccagtacaactacgtggacgctgtgtgctatgagaaccgactgcactggtttgccaagtacttcccctacctggtgcttctgcacacgctcatcttcctggcctgcagcaacttctggttcaaattcccgcgcaccagctcgaagctggagcactttgtgtctatcctgctgaagtgcttcgactcgccctggaccacgagggccctgtcggagacagtggtggaggagagcgaccccaagccggccttcagcaagatgaatgggtccatggacaaaaagtcatcgaccgtcagtgaggacgtggaggccaccgtgcccatgctgcagcggaccaagtcacggatcgagcagggtatcgtggaccgctcagagacgggcgtgctggacaagaaggagggggagcaagccaaggcgctgtttgagaaggtgaagaagttccggacccatgtggaggagggggacattgtgtaccgcctctacatgcggcagaccatcatcaaggtgatcaagttcatcctcatcatctgctacaccgtctactacgtgcacaacatcaagttcgacgtggactgcaccgtggacattgagagcctgacgggctaccgcacctaccgctgtgcccaccccctggccacactcttcaagatcctggcgtccttctacatcagcctagtcatcttctacggcctcatctgcatgtatacactgtggtggatgctacggcgctccctcaagaagtactcgtttgagtcgatccgtgaggagagcagctacagcgacatccccgacgtcaagaacgacttcgccttcatgctgcacctcattgaccaatacgacccgctctactccaagcgcttcgccgtcttcctgtcggaggtgagtgagaacaagctgcggcagctgaacctcaacaacgagtggacgctggacaagctccggcagcggctcaccaagaacgcgcaggacaagctggagctgcacctgttcatgctcagtggcatccctgacactgtgtttgacctggtggagctggaggtcctcaagctggagctgatccccgacgtgaccatcccgcccagcattgcccagctcacgggcctcaaggagctgtggctctaccacacagcggccaagattgaagcgcccgcgctggccttcctgcgcgagaacctgcgggcgctgcacatcaagttcaccgacatcaaggagatcccgctgtggatctatagcctgaagacactggaggagctgcacctgacgggcaacctgagcgcggagaacaaccgctacatcgtcatcgacgggctgcgggagctcaaacgcctcaaggtgctgcggctcaagagcaacctaagcaagctgccacaggtggtcacagatgtgggcgtgcacctgcagaagctgtccatcaacaatgagggcaccaagctcatcgtcctcaacagcctcaagaagatggcgaacctgactgagctggagctgatccgctgtgacctggagcgcatcccccactccatcttcagcctccacaacctgcaggagattgacctcaaggacaacaacctcaagaccatcgaggagatcatcagcttccagcacctgcaccgcctcacctgccttaagctgtggtacaaccacatcgcctacatccccatccagatcggcaacctcaccaacctggagcgcctctacctgaaccgcaacaagatcgagaagatccccacccagctcttctactgccgcaagctgcgctacctggacctcagccacaacaacctgaccttcctccctgccgacatcggcctcctgcagaacctccagaacctagccatcacggccaaccggatcgagacgctccctccggagctcttccagtgccggaagctgcgggccctgcacctgggcaacaacgtgctgcagtcactgccctccagggtgggcgagctgaccaacctgacgcagatcgagctgcggggcaaccggctggagtgcctgcctgtggagctgggcgagtgcccactgctcaagcgcagcggcttggtggtggaggaggacctgttcaacacactgccacccgaggtgaaggagcggctgtggagggctgacaaggagcaggcctgagcgaggccggcccagcacagcaagcagcaggaccgctgcccagtcctcaggcccggaggggcaggcctagcttctcccagaactcccggacagccaggacagcctcgtggctgggcaggagcctggggccgcttgtgagtcaggccagagcgagaggacagtatctgtggggctggccccttttctccctctgagactcacgtcccccagggcaagtgcttgtggaggagagcaagtctcaagagcgcagtatttggataatcagggtctcctccctggaggccagctctgccccaggggctgagctgccaccagaggtcctgggaccctcactttagttcttggtatttatttttctccatctcccacctccttcatccagataacttatacattcccaagaaagttcagcccagatggaaggtgttcagggaaaggtgggctgccttttccccttgtccttatttagcgatgccgccgggcatttaacacccacctggacttcagcagagtggtccggggcgaaccagccatgggacggtcacccagcagtgccgggctgggctctgcggtgcggtccacgggagagcaggcctccagctggaaaggccaggcctggagcttgcctcttcagtatttgtggcagttttagttttttgtttttttttttttaatcaaaaaacaatttttttaaaaaaaaagctttgaaaatggatggtttgggtattaaaaagaaaaaaaaaacttaaaaaaaaaaagacactaacggccagtgagttggagtctcagggcagggtggcagtttcccttgagcaaagcagccagacgttgaactgtgtttcctttccctgggcgcagggtgcagggtgtcttccggatctggtgtgaccttggtccaggagttctatttgttcctggggagggaggtttttttgtttgttttttgggtttttttggtgtcttgttttctttctcctccatgtgtcttggcaggcactcatttctgtggctgtcggccagagggaatgttctggagctgccaaggagggaggagactcgggttggctaatccccggatgaacggtgctccattcgcacctcccctcctcgtgcctgccctgcctctccacgcacagtgttaaggagccaagaggagccacttcgcccagactttgtttccccaccgcctgcggcatgggtgtgtccagtgccaccgctggcctccgctgcttccatcagccctgtcgccacctggtccttcatgaagagcagacacttagaggctggtcgggaatggggaggtcgcccctgggagggcaggcgttggttccaagccggttcccgtccctggcgcctggagtgcacacagcccagtcggcacctggtggctggaagccaccctgctttagatcactcgggtccccaccttagaagggtccccgccttagatcaatcacgtggacactaaggcacgttttagagtctcttgtcttaatgattatgtccatccgtctgtccgtccatttgtgttttctgcgtcgtgtcattggatataatcctcagaaataatgcacactagcctctgacaaccatgaagcaaaaatccgttacatgtgggtctgaacttgtagactcggtcacagtatcaaataaaatctataacagaaaaaaaaaaaaaaaa

SEQ ID NO: 6 - DNA, 3501 bp, Artificial Sequence
  LRRC8A_reg seq#1
gcctgtctgaggagatctaacaattgcgtcactcagacacagtccccctgggcttaggaattggcagaggagagagaaatgacttctggagtccagacggcaggtttacaggctggaggggttggtggaagacctgagtgacacaaacagtaggtatctcaccacttattcctcctggcacaggacccagattcaaaaaccacaatctcactcccatacattaaaggaacacacaacagataggcagatagcctcgcgggagccttggaggaggtggcatcagctttacaggtggggaaactgaggcttagagaggggaaatgacttactcgaagtcccagaaaagcaccaggaggcccccagctgcaagctggggaatgagggcataggatgagcattttaaaaattatgccgggaggccgggcgcggtggctcacgcctgtaatcccagcactttgggaggccgaggtgggcggatcacaaggtcaggagatagagaccatcctggctaacacggtgaaaccccgtctctactaaaaatacaaaaaattagccaggcgtggtggcgggtgcctgtagtcccagctacttgggaggctgaggcaggagaatggagtgaacccgggaggtggagcttgcagtgagccgagattgcaccactgcactccagcctgggcgacagagtgagactccgtctcaaaaaaaaaaaaaggaattatgcagggaactggcagtgccatccaggagtgggatgtggccccaggtctgttctggcaggagttacagtgactgcctcatggctgtgaaggctcctgcattcctttatcgcccccacctggattataaaatcaggcgccctactctttggagcttggagaggttagtgtggcgttctggtggttggcacctaatggaggcattctccgggaggaaagattcctgctgaagccagactgaatgggtctaacttgtatattctccaatcctgttggtcaccctcagtttcttagtggacaccaaggccaagtccagggtaggagcccatctctccttgagtctccagcctcatcatttctccttctggacatcctagtttccagtcacattgaaagtcccttccttggagaagccccttctgactccacccaggctaagccacatgtcccctcccaggtggtcccaagacctgggcttccgccctcaaagtactcagcacgggggcgctcaactccaaggacaggaactatgtgcttgacctgttctccaccagatcctcagcctctggcagctcacccagcacagagtaggtcccccaggcattagcacagatgatcctgggaagctaagggagaggcctgggagcccagcagccttcctagggagggagggagacctgtaggaacccccttctggcaactttcttaatctctctgtgcctcacttatctgtaaaatgagacttatctgcgtgtaaaatacaaagtataatacgtgggaacattcactgtctacaactgtggttctggttgttcattttacaggcggggacatggaggcccacggagtacctggcaggcccacagtccacaggttggaaagaggtgcccaagccctggactttaagcctgggctctgaccttcaacgtttgcttttcacaccacacatcatgtcaataaatagttactggatgcctgttgtgtgccaggccccaagacgggtgttgcgtgactgacaacagaaaaagcatctcagtggcgagggataagctagatcatgggcattagtgtaaatctcccagggttcaaattccagcttctccacttcctggctatgatttttttttttttttagacggagtcttgctctgttgcccaggctgaagtgcagtggcccaatctcagctcactgcaagctctgcctcccaggttcatgccattctcctgcctcagcctcccgagtagctgggaccacaggcgctcgccaccacacccggctaatttttttgtatttttagtagagacggggtttcaccgtgttagccaggatggtctcgatctactgacctcgtgatccgcccgcctcagcctcccaaagtgctgggattacaggtgtgaaccaccgcgcccggccctggctatgatattgctaagtttcctaacctctctaaggttcttttctcttctattaaaagggaaaaataagacctcccaacagaagtgaaactaggcattggcttaacacgtgatggatgggagccacgactatcttcatcatcttatttttaattatctatctgtgcacatggtagtaaatttctcgccctggcccaggtacccaagaccccaattctgtcggagacaaccgcaattaacaacttcttgtgcatcttatcagacagctggcgcgcacacccgcagatacgcccaccctcaggcgtccttctacgcaaagatggatagcacccatcaacgctgttttttctttttttagacggagtctcgctctgtcgcccaggctggagtgcagcggcgcgatctcggctcactgcaagctccacctcccgagttcatgccattctcctgcctcagcctccagagtagctgggactacaggcgcccaccaccacgcccggctgatttttttgtatttttagtagagatgaggtttcaccaagttagccaggatggtctcgatctcctgacctcgtgatccgcccgcctcggcctcccaaagtgctgggattacagacgtgagccaccgcaaccggcccaacgctgtttttactgttgtttccttttttaacctaaggtatcttagaagtcaccctacattagtgcatattaaacattgttcgtcttaatggctgcacagtattccactgtatggattattttttaatttggttgccggtactgttatcagccagggaatttgcgaggacccagcgggatttatggagctgcagagttggagtctggggacccaattccaaccccagatctgtccagaattctgttgtctcgggtatcatctccaccggcgcggtcgtgggagggggatttgggtgcagacaggaccagccccagtgtccgagcgagaatcagcgagcagcacgcgcagttgattcccgcgggcggctcaatcattctgtgcagccactccgttatacttagtagcatcaaactcattaagcactctagcacaaccaagctcggaaccgttagccgaccctcccagcctcggcacctgaccttggggaagtggcgctccgacacagctactcaccctcacctgggccgcttcacccctcgttcccaagtacaccccgtaggttgcagcagtccctgtccctttaagggggccgagcccggctccgctacttccgccccgaagcagcagggcgctagcgcggaggcgagagcgggagaaagcgccgctagaattctcctcataaagatggcgacgccctggccgccgcgttcgcgcccggcggtgacgtca

SEQ ID NO: 7 - DNA, 3501 bp, Artificial Sequence
  LRRC8A_reg seq#2
atgagcattttaaaaattatgccgggaggccgggcgcggtggctcacgcctgtaatcccagcactttgggaggccgaggtgggcggatcacaaggtcaggagatagagaccatcctggctaacacggtgaaaccccgtctctactaaaaatacaaaaaattagccaggcgtggtggcgggtgcctgtagtcccagctacttgggaggctgaggcaggagaatggagtgaacccgggaggtggagcttgcagtgagccgagattgcaccactgcactccagcctgggcgacagagtgagactccgtctcaaaaaaaaaaaaaggaattatgcagggaactggcagtgccatccaggagtgggatgtggccccaggtctgttctggcaggagttacagtgactgcctcatggctgtgaaggctcctgcattcctttatcgcccccacctggattataaaatcaggcgccctactctttggagcttggagaggttagtgtggcgttctggtggttggcacctaatggaggcattctccgggaggaaagattcctgctgaagccagactgaatgggtctaacttgtatattctccaatcctgttggtcaccctcagtttcttagtggacaccaaggccaagtccagggtaggagcccatctctccttgagtctccagcctcatcatttctccttctggacatcctagtttccagtcacattgaaagtcccttccttggagaagccccttctgactccacccaggctaagccacatgtcccctcccaggtggtcccaagacctgggcttccgccctcaaagtactcagcacgggggcgctcaactccaaggacaggaactatgtgcttgacctgttctccaccagatcctcagcctctggcagctcacccagcacagagtaggtcccccaggcattagcacagatgatcctgggaagctaagggagaggcctgggagcccagcagccttcctagggagggagggagacctgtaggaacccccttctggcaactttcttaatctctctgtgcctcacttatctgtaaaatgagacttatctgcgtgtaaaatacaaagtataatacgtgggaacattcactgtctacaactgtggttctggttgttcattttacaggcggggacatggaggcccacggagtacctggcaggcccacagtccacaggttggaaagaggtgcccaagccctggactttaagcctgggctctgaccttcaacgtttgcttttcacaccacacatcatgtcaataaatagttactggatgcctgttgtgtgccaggccccaagacgggtgttgcgtgactgacaacagaaaaagcatctcagtggcgagggataagctagatcatgggcattagtgtaaatctcccagggttcaaattccagcttctccacttcctggctatgatttttttttttttttagacggagtcttgctctgttgcccaggctgaagtgcagtggcccaatctcagctcactgcaagctctgcctcccaggttcatgccattctcctgcctcagcctcccgagtagctgggaccacaggcgctcgccaccacacccggctaatttttttgtatttttagtagagacggggtttcaccgtgttagccaggatggtctcgatctactgacctcgtgatccgcccgcctcagcctcccaaagtgctgggattacaggtgtgaaccaccgcgcccggccctggctatgatattgctaagtttcctaacctctctaaggttcttttctcttctattaaaagggaaaaataagacctcccaacagaagtgaaactaggcattggcttaacacgtgatggatgggagccacgactatcttcatcatcttatttttaattatctatctgtgcacatggtagtaaatttctcgccctggcccaggtacccaagaccccaattctgtcggagacaaccgcaattaacaacttcttgtgcatcttatcagacagctggcgcgcacacccgcagatacgcccaccctcaggcgtccttctacgcaaagatggatagcacccatcaacgctgttttttctttttttagacggagtctcgctctgtcgcccaggctggagtgcagcggcgcgatctcggctcactgcaagctccacctcccgagttcatgccattctcctgcctcagcctccagagtagctgggactacaggcgcccaccaccacgcccggctgatttttttgtatttttagtagagatgaggtttcaccaagttagccaggatggtctcgatctcctgacctcgtgatccgcccgcctcggcctcccaaagtgctgggattacagacgtgagccaccgcaaccggcccaacgctgtttttactgttgtttccttttttaacctaaggtatcttagaagtcaccctacattagtgcatattaaacattgttcgtcttaatggctgcacagtattccactgtatggattattttttaatttggttgccggtactgttatcagccagggaatttgcgaggacccagcgggatttatggagctgcagagttggagtctggggacccaattccaaccccagatctgtccagaattctgttgtctcgggtatcatctccaccggcgcggtcgtgggagggggatttgggtgcagacaggaccagccccagtgtccgagcgagaatcagcgagcagcacgcgcagttgattcccgcgggcggctcaatcattctgtgcagccactccgttatacttagtagcatcaaactcattaagcactctagcacaaccaagctcggaaccgttagccgaccctcccagcctcggcacctgaccttggggaagtggcgctccgacacagctactcaccctcacctgggccgcttcacccctcgttcccaagtacaccccgtaggttgcagcagtccctgtccctttaagggggccgagcccggctccgctacttccgccccgaagcagcagggcgctagcgcggaggcgagagcgggagaaagcgccgctagaattctcctcataaagatggcgacgccctggccgccgcgttcgcgcccggcggtgacgtcacttccggggcggaggaggctgagtggtgcagtgagggacaaacaaaaggaggcgccggagcagcgctgcggccggcggcgggacggagcggccggggcctggggctgcctgccgggcggccgggcgcggcgagcccaggtgagtggacggggtggggaaaggggcgcgagctgtcacctctcgaaacccacttacacacgccctcggccagctgcccggcccggggcgcccggggcatctggggctctgtctccgggccggccccctggggacctcccgcggtgttcggcctacctctgggctccccagtgcccggagtctccggggcaccaccttctcgccggctccgcggcgcgcgagccccctcccaggcacccctgtgcctccttg

SEQ ID NO: 8 - DNA, 20 bp, Artificial Sequence
  keratin 1 (KRT1) fw primer
tgagccgcattctgaacgag

SEQ ID NO: 9 - DNA, 20 bp, Artificial Sequence
  keratin 1 (KRT1) rv primer
gatgactgcgatccagagga

SEQ ID NO: 10 - DNA, 20 bp, Artificial Sequence
  keratin 10 (KRT10) fw primer
ggtgggagttatggaggcag

SEQ ID NO: 11 - DNA, 23 bp, Artificial Sequence
  keratin 10 (KRT10) rv primer
cgaactttgtccaagtaggaagc

SEQ ID NO: 12 - DNA, 21 bp, Artificial Sequence
  filaggrin (FLG) fw primer
gcactcgtcatgcagagactt

SEQ ID NO: 13 - DNA, 19 bp, Artificial Sequence
  filaggrin (FLG) rv primer
gaccctcggtttccactgt

SEQ ID NO: 14 - DNA, 21 bp, Artificial Sequence
  loricrin (LOR) fw primer
ctcctgtgggttgtggaaaga

SEQ ID NO: 15 - DNA, 21 bp, Artificial Sequence
  loricrin (LOR) rv primer
tggaaccacctccataggaac

SEQ ID NO: 16 - DNA, 21 bp, Artificial Sequence
  keratin 5 (KRT5) fw primer
aggagttggaccagtcaacat

SEQ ID NO: 17 - DNA, 21 bp, Artificial Sequence
  keratin 5 (KRT5) rv primer
tggagtagtagcttccactgc

SEQ ID NO: 18 - DNA, 20 bp, Artificial Sequence
  keratin 14 (KRT14) fw primer
tgagccgcattctgaacgag

SEQ ID NO: 19 - DNA, 20 bp, Artificial Sequence
  keratin 14 (KRT14) rv primer
gatgactgcgatccagagga

SEQ ID NO: 20 - DNA, 20 bp, Artificial Sequence
  S100 calcium binding protein A8 (S100A8) fw primer
atgccgtctacagggatgac

SEQ ID NO: 21 - DNA, 21 bp, Artificial Sequence
  S100 calcium binding protein A8 (S100A8) rv primer
actgaggacactcggtctcta

SEQ ID NO: 22 - DNA, 23 bp, Artificial Sequence
  S100 calcium binding protein A9 (S100A9) fw primer
ggtcatagaacacatcatggagg

SEQ ID NO: 23 - DNA, 19 bp, Artificial Sequence
  S100 calcium binding protein A9 (S100A9) rv primer
ggcctggcttatggtggtg

SEQ ID NO: 24 - DNA, 19 bp, Artificial Sequence
  serpin family B member 3 (SERPINB3) fw primer
cgcggtctcgtgctatctg

SEQ ID NO: 25 - DNA, 21 bp, Artificial Sequence
  serpin family B member 3 (SERPINB3) rv primer
atccgaatcctactacagcgg

SEQ ID NO: 26 - DNA, 21 bp, Artificial Sequence
  serpin family B member 4 (SERPINB4) fw primer
ctgggtggaaagtcaaacgaa

SEQ ID NO: 27 - DNA, 23 bp, Artificial Sequence
  serpin family B member 4 (SERPINB4) rv primer
tgtcgtatcattgccaatagtcc

SEQ ID NO: 28 - DNA, 21 bp, Artificial Sequence
  peptidase inhibitor 3 (PI3) fw primer
cacgggagttcctgttaaagg

SEQ ID NO: 29 - DNA, 21 bp, Artificial Sequence
  peptidase inhibitor 3 (PI3) rv primer
tctttcaagcagcggttaggg

SEQ ID NO: 30 - DNA, 23 bp, Artificial Sequence
  lipocalin 2 (LCN2) fw primer
gaagtgtgactactggatcagga

SEQ ID NO: 31 - DNA, 20 bp, Artificial Sequence
  lipocalin 2 (LCN2) rv primer
accactcggacgaggtaact

SEQ ID NO: 32 - DNA, 22 bp, Artificial Sequence
  transglutaminase 1 (TGM1) fw primer
atcatcggcaagtttcagttca

SEQ ID NO: 33 - DNA, 23 bp, Artificial Sequence
  transglutaminase 1 (TGM1) rv primer
tcccgtagtaaattctcccagac

SEQ ID NO: 34 - DNA, 19 bp, Artificial Sequence
  keratin 16 (KRT16) fw primer
gaccggcggagatgtgaac

SEQ ID NO: 35 - DNA, 19 bp, Artificial Sequence
  keratin 16 (KRT16) rv primer
ctgctcgtactggtcacgc

SEQ ID NO: 36 - DNA, 22 bp, Artificial Sequence
  defensin Beta 1 (DEFB1) fw primer
agacttgtgctgctattagccg

SEQ ID NO: 37 - DNA, 21 bp, Artificial Sequence
  defensin Beta 1 (DEFB1) rv primer
gggcagtcccataaccacata

SEQ ID NO: 38 - DNA, 21 bp, Artificial Sequence
  involucrin (IVL) fw primer
gactgctgtaaagggactgcc

SEQ ID NO: 39 - DNA, 22 bp, Artificial Sequence
  involucrin (IVL) rv primer
cattcccagttgctcatctctc

SEQ ID NO: 40 - DNA, 23 bp, Artificial Sequence
  S100 calcium binding protein A7 (S100A7) fw primer
acgtgatgacaagattgacaagc

SEQ ID NO: 41 - DNA, 21 bp, Artificial Sequence
  S100 calcium binding protein A7 (S100A7) rv primer
gcgaggtaatttgtgcccttt

SEQ ID NO: 42 - DNA, 20 bp, Artificial Sequence
  beta-Actin fw primer
gtggggcgccccaggcacca

SEQ ID NO: 43 - DNA, 24 bp, Artificial Sequence
  beta-Actin rv primer
ctccttaatgtcacgcacgatttc

SEQ ID NO: 44 - DNA, 21 bp, Artificial Sequence
  LRRC8A fw primer
cctgccttgtaagtgggtcac

SEQ ID NO: 45 - DNA, 21 bp, Artificial Sequence
  LRRC8A rv primer
cacagcgtccacgtagttgta

SEQ ID NO: 46 - DNA, 21 bp, Artificial Sequence
  LRRC8B fw primer
ctggcatagaaagcccaactt

SEQ ID NO: 47 - DNA, 22 bp, Artificial Sequence
  LRRC8B rv primer
cgatttcaagagtgatgtgggt

SEQ ID NO: 48 - DNA, 22 bp, Artificial Sequence
  LRRC8C fw primer
ctggggaagtgttttgactctc

SEQ ID NO: 49 - DNA, 21 bp, Artificial Sequence
  LRRC8C rv primer
ggaccagattggatggtgttg

SEQ ID NO: 50 - DNA, 22 bp, Artificial Sequence
  LRRC8D fw primer
gtggtctgtttgccagtattgc

SEQ ID NO: 51 - DNA, 23 bp, Artificial Sequence
  LRRC8D rv primer
cccaaaggaaatgtcgtttgttg

SEQ ID NO: 52 - DNA, 20 bp, Artificial Sequence
  LRRC8E fw primer
caagcagttcacggaacagc

SEQ ID NO: 53 - DNA, 22 bp, Artificial Sequence
  LRRC8E rv primer
gggcctctgataagttctcctg

SEQ ID NO: 54 - DNA, 20 bp, Artificial Sequence
  sgRNA-G-LRRC8A#1
gctgcgtgtccgcaaagtag

SEQ ID NO: 55 - DNA, 20 bp, Artificial Sequence
  sgRNA-G-LRRC8A#4
ccggcaccagtacaactacg

SEQ ID NO: 56 - DNA, 18 bp, Artificial Sequence
  Seq_LRRC8A_24839 fw primer
tggtttcccagccaagtg

SEQ ID NO: 57 - DNA, 20 bp, Artificial Sequence
  Seq_LRRC8A_965 rv primer
gcgggaatttgaaccagaag

(19) United States
(12) Patent Application Publication
(10) Pub. No.: US 2020/0393448 A1
(43) Pub. Date: Dec. 17, 2020

(54) THE VOLUME-REGULATED ANION CHANNEL PROTEIN LRRC8A FOR USE IN ALTERING EPIDERMAL KERATINOCYTE DIFFERENTIATION

(71) Applicant: B.R.A.I.N. Biotechnology Research and Information Network AG, Zwingenberg (DE)

(72) Inventors: Torsten ERTONGUR-FAUTH, Darmstadt (DE); Janina TROTHE, Bovenden (DE); Claudia BÜRGER, Frankfurt (DE)

(21) Appl. No.: 16/969,541

(22) PCT Filed: Feb. 15, 2019
(86) PCT No.: PCT/EP2019/053820

§ 371 Date: Aug. 12, 2020

(30) Foreign Application Priority Data
Feb. 16, 2018 (EP) 18157265.2

(51) Int. Cl. G01N 33/50; A61K 38/17; A61P 17/06; C12N 9/22; A61K 8/64; A61Q 19/00

(52) U.S. Cl. CPC G01N 33/5044; A61K 38/1709; A61P 17/06; G01N 2800/202; A61K 8/64; A61Q 19/00; G01N 2333/4742; C12N 9/22

(57) ABSTRACT

The present invention relates to the leucine-rich repeat-containing protein 8A (LRRC8A), and/or an activator of LRRC8A, for use in the treatment and/or prevention of a skin condition associated with an altered differentiation of keratinocytes. Preferably, the skin condition associated with an altered differentiation of keratinocytes is psoriasis or dermatitis, preferably atopic dermatitis. The present invention further relates to a method of identifying a compound capable of altering the differentiation of keratinocytes, the method comprising the steps of (a) contacting keratinocytes with a test compound and determining the amount of LRRC8A protein or LRRC8A transcript in said keratinocytes; and (b) comparing the amount of LRRC8A protein or LRRC8A transcript determined in step (a) with the amount of LRRC8A protein or LRRC8A transcript in a control not contacted with said test compound, wherein a change in the amount of LRRC8A protein or LRRC8A transcript after contacting the keratinocytes with the test compound indicates that the test compound is capable of altering the differentiation of keratinocytes. Furthermore, the present invention relates to a method of identifying a compound capable of altering the differentiation of keratinocytes, the method comprising the steps of (a) contacting keratinocytes with a test compound and determining the activity of (a) VRAC(s) comprising LRRC8A in said keratinocytes; and (b) comparing the activity determined in step (a) with the activity in a control not contacted with said test compound, wherein a change in the activity of (a) VRAC(s) comprising LRRC8A after contacting the keratinocytes with the test compound indicates that the test compound is capable of altering the differentiation of keratinocytes. The present invention further relates to an inhibitor of the leucine-rich repeat-containing protein 8A (LRRC8A) for use in the treatment and/or prevention of a skin condition selected from skin injury and impaired wound healing, as well as to a cosmetic method for alleviating the effects of a skin condition on the appearance of the skin of an affected individual, the method comprising topically administering an effective amount of (i) leucine-rich repeat-containing protein 8A (LRRC8A); (ii) an activator of LRRC8A; (iii) LRRC8A and an activator of LRRC8A; or (iv) an inhibitor of LRRC8A.

### RELATED PATENT APPLICATION

This patent application is a 35 U.S.C. 371 national phase patent application of PCT/EP2019/053820 filed on Feb. 15, 2019, entitled “THE VOLUME-REGULATED ANION CHANNEL PROTEIN LRRC8A FOR USE IN ALTERING EPIDERMAL KERATINOCYTE DIFFERENTIATION”, naming Torsten Ertongur-Fauth et al. as inventors, and designated by attorney docket no. AA2153 PCT which claims priority to European Application No. 18157265.2 filed on Feb. 16, 2018, entitled “THE VOLUME-REGULATED ANION CHANNEL PROTEIN LRRC8A FOR USE IN ALTERING EPIDERMAL KERATINOCYTE DIFFERENTIATION,” naming Torsten Ertongur-Fauth et al. as inventors, and designated by attorney docket no. AA2153 EP. The entire content of the foregoing patent applications is incorporated herein by reference, including all text, tables and drawings.

### SEQUENCE LISTING

The instant application contains a Sequence Listing which has been submitted electronically in ASCII format and is hereby incorporated by reference in its entirety. Said ASCII copy is named Sequence Listing and is 47 kilobytes in size.

The present invention relates to the leucine-rich repeat-containing protein 8A (LRRC8A), and/or an activator of LRRC8A, for use in the treatment and/or prevention of a skin condition associated with an altered differentiation of keratinocytes. Preferably, the skin condition associated with an altered differentiation of keratinocytes is psoriasis or dermatitis, preferably atopic dermatitis. The present invention further relates to a method of identifying a compound capable of altering the differentiation of keratinocytes, the method comprising the steps of (a) contacting keratinocytes with a test compound and determining the amount of LRRC8A protein or LRRC8A transcript in said keratinocytes; and (b) comparing the amount of LRRC8A protein or LRRC8A transcript determined in step (a) with the amount of LRRC8A protein or LRRC8A transcript in a control not contacted with said test compound, wherein a change in the amount of LRRC8A protein or LRRC8A transcript after contacting the keratinocytes with the test compound indicates that the test compound is capable of altering the differentiation of keratinocytes. Furthermore, the present invention relates to a method of identifying a compound capable of altering the differentiation of keratinocytes, the method comprising the steps of (a) contacting keratinocytes with a test compound and determining the activity of (a) VRAC(s) comprising LRRC8A in said keratinocytes; and (b) comparing the activity determined in step (a) with the activity in a control not contacted with said test compound, wherein a change in the activity of (a) VRAC(s) comprising LRRC8A after contacting the keratinocytes with the test compound indicates that the test compound is capable of altering the differentiation of keratinocytes. The present invention further relates to an inhibitor of the leucine-rich repeat-containing protein 8A (LRRC8A) for use in the treatment and/or prevention of a skin condition selected from skin injury and impaired wound healing, as well as to a cosmetic method for alleviating the effects of a skin condition on the appearance of the skin of an affected individual, the method comprising topically administering an effective amount of (i) leucine-rich repeat-containing protein 8A (LRRC8A); (ii) an activator of LRRC8A; (iii) LRRC8A and an activator of LRRC8A; or (iv) an inhibitor of LRRC8A.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The main protective function of the human skin is achieved by the epidermis, which is composed of several layers of differentiating keratinocytes. To maintain homeostasis of the healthy epidermis, keratinocytes develop gradually from proliferating basal cells into spinous, granular and corneal layers. This differentiation process involves ordered gene expression changes that lead to drastic morphological and functional changes of the keratinocytes. This balance between keratinocyte proliferation and differentiation is tightly regulated in healthy skin, but is disturbed in skin diseases such as psoriasis or atopic dermatitis.

The human epidermis not only forms an important barrier against detrimental environmental influences, it also plays a fundamental role in water homeostasis of the skin: it contributes to maintaining the hydration state of the body by preventing trans-epidermal water loss and it protects against environmental osmotic fluctuations¹. However, the epidermal barrier function is impaired in certain diseases such as psoriasis, atopic dermatitis or eczema and the underlying epidermal keratinocytes become a direct target of osmotic stress².

Psoriasis is a chronic inflammatory skin disease presenting with red scaly plaques, mostly on the head, trunk and extensor sites of arms and legs and is associated with a physical and psychological burden. Symptoms include pain, itch, and bleeding. The severity of the disease is often increased by co-morbid diseases, such as metabolic syndrome or cardiovascular diseases³. The Psoriasis Area and Severity Index (PASI) score is used to quantify disease severity by estimating the degree of erythema, infiltration or thickness, scaling and the extent of lesions. A maximum of 72 can be reached and a PASI above 10 is considered moderate-to-severe.

Various in vitro psoriasis models are known and are commonly employed to study signaling pathways, transcriptional regulation, inflammation, differentiation and proliferation on the molecular and cellular level in psoriasis⁴⁻⁶. In addition, these models can also be used to judge the effect of new compounds or therapies for the treatment of psoriasis. Typically, such models are generated by treating normal human keratinocytes (NHK) with a cocktail of psoriatic cytokines, including e.g. TNF-α and IL-17, thereby inducing the aberrant differentiation of keratinocytes characteristic of psoriasis. Normalization of said aberrant differentiation, for example after treatment with relevant compounds, is typically analysed based on changes in the expression levels of known differentiation markers. Thus, an increased expression of the markers keratin 1 (KRT1), keratin 10 (KRT10), filaggrin (FLG) or loricrin (LOR) as compared to their expression level in in vitro psoriatic model keratinocytes that were not treated with said compound, is typically considered indicative of a normalization of aberrant differentiation.

Psoriasis affects multiple cell types such as T-cells, neutrophils, macrophages and keratinocytes in the skin. Further complexity arises from the different severities of the disease and in-between-patient-variations. Despite these complexities, a set of genes commonly affected in psoriatic keratinocytes and in in vitro keratinocyte models of psoriasis can be extracted as marker genes from published transcriptome studies^{5,7}. This set of genes include genes of the S100A group (e.g. S100 calcium-binding proteins S100A8 and S100A9), CXCL genes (e.g. chemokine CXC motif ligands CXCL1 or CXCL8/IL-8), small proline-rich protein 2 group (e.g. SPRR2C or SPRR2D), serpin peptidase inhibitors Glade B (e.g. SERPINB3 or SERPINB4), skin-derived peptidase inhibitor 3 (PI3), lipocalin 2 (LCN2) and transglutaminase type I (TGM1), all of which are upregulated in psoriasis. In addition, the expression of keratins KRT1 and KRT10 has been found to be reduced, while the markers for hyperproliferation KRT6 and KRT16 are upregulated in psoriatic epidermis⁸.

So far, five types of psoriasis have been reported: plaque psoriasis (also known as psoriasis vulgaris); guttate (droplet), which is characterized by scaly teardrop-shaped spots; inverse psoriasis, that is usually found in folds of skin; pustular psoriasis, which can either take the form of palmoplantar pustulosis (pustular psoriasis of the palms and soles), or generalised pustular psoriasis (a rare and serious form of psoriasis); and erythrodermic psoriasis, which is a rare but very serious complication of psoriasis³.

Patients with psoriasis suffering from mild disease are typically treated with topical therapies using agents such as corticosteroids, vitamin D analogues, topical retinoids and calcineurin inhibitors. For moderate-to-severe psoriasis, for example psoriasis affecting large surface areas, a well-established treatment regimen consists of a combination of topical agents and phototherapy or systemic drugs^{9,10}. Systemic drugs include methotrexate, ciclosporin, acitretin and, in some countries, fumaric acid esters, which are given orally. In addition, several biologics have been developed in the past decade, which mainly consist of antibodies that target TNF-α, IL-17A or IL-12/IL-23³. However, a major drawback of using these drugs is that they either need intravenous infusion or sub-cutaneous injection.

Also eczema, and in particular atopic dermatitis (AD), are skin disorders in which keratinocyte proliferation is enhanced, whereas differentiation is disturbed, as can be observed for example by increased KRT6 and reduced KRT10 expression in lesional skin of AD¹¹.

Patients suffering from eczema and AD are typically advised to routinely use emollients during bathing to hydrate the affected skin. In addition, patients receive topical treatment with steroids, such as e.g. hydrocortisone, or with calcineurin inhibitors that lead to down-stream inhibition of cytokine expression in T-cells and, thereby, reducing skin inflammation. For moderate-to-severe AD, antibodies targeting interleukin signaling can be used. Newer strategies, which are currently being tested in clinical trials, focus on targeting the JAK-STAT pathway by using both orally and topically applied small-molecule JAK-1/2 inhibitors¹². However, the JAK-STAT pathway is a conserved master regulator of immunity and, thus, the suitability of strategies that target such a central play will have to be critically reviewed, for example in large safety and efficacy trials that will have to be performed before these strategies can reach the market¹³.

Despite the fact that a lot of effort is currently being invested into the characterisation of these diseases, it is remarkable that no novel substances have been developed for topical treatment of psoriasis in the last years. In addition, all newly introduced agents have been mainly analogues, derivatives or new formulations of already known agents¹⁴.

Accordingly, there is still a need to provide novel approaches for the treatment of these skin disorders and for alleviating the effects of these skin conditions on the appearance of the skin of an affected individual. Moreover, methods for the identification of novel agents are urgently required. Such methods would represent valuable research tools and would offer tremendous value to the field.

This need is addressed by the provision of the embodiments characterised in the claims.

Accordingly, the present invention relates to the leucine-rich repeat-containing protein 8A (LRRC8A), and/or an activator of LRRC8A, for use in the treatment and/or prevention of a skin condition associated with an altered differentiation of keratinocytes.

As used herein, the term “leucine-rich repeat-containing protein 8A” refers to a protein belonging to the leucine-rich repeat family of proteins, which are involved in diverse biological processes, for example lymphocyte development and cell volume regulation¹⁵. The leucine-rich repeat-containing protein 8A is abbreviated herein as LRRC8A. This leucine-rich repeat family of proteins is a family of ion channel proteins that includes LRRC8A to LRRC8F, all of which share a conserved domain structure encompassing four transmembrane domains and a C-terminal domain containing 17 leucine-rich repeats¹⁶. Six subunits of this protein family are required to form a functional volume-regulated anion channel (VRAC) and LRRC8A has been described to assemble into heteromeric complexes with at least one additional LRRC8 subunit^{17,18.} Accordingly, it is also envisaged in accordance with the present invention that LRRC8A is provided for the inventive use in combination with at least one further LRRC8 subunit selected from LRRC8A, LRRC8B, LRRC8C, LRRC8D, LRRC8E and LRRC8F. Further encompassed is that LRRC8A is provided as an LRRC8 complex of six subunits comprising at least one subunit that is LRRC8A.

Human LRRC8A is represented, for example, by the RefSeq Gene ID 56262, as updated on Nov. 23, 2017 and the UniProtKB accession number Q8IWT6, as updated on Nov. 22, 2017. Human LRRC8A is also shown in SEQ ID NOs: 1 and 2.

The term “activator”, as used herein, is defined as a compound inducing or enhancing the expression and/or activity of a target molecule, i.e. of LRRC8A. Preferably, the activator mediates one or more of the following effects: (i) the expression, i.e. transcription and/or translation, of the gene encoding LRRC8A is induced or increased, and (ii) LRRC8A performs its function, such as e.g. its biochemical and/or cellular function, with increased efficiency in the presence of the activator.

Compounds falling in class (i) include compounds interacting with the transcriptional machinery and/or with the promoter of the LRRC8A gene and/or with expression control elements remote from the promoter such as enhancers. Also included are antisense constructs and constructs for performing RNA interference (e.g. siRNA, shRNA, miRNA) well known in the art (see, e.g. Zamore (2001) Nat. Struct. Biol. 8(9), 746; Tuschl (2001) Chembiochem. 2(4), 239), targeted to molecules that e.g. inhibit LRRC8A expression. Compounds falling in class (i) include compounds that have a directly activating effect on LRRC8A expression but also molecules that are indirectly activating, e.g. by interacting for example with molecules that regulate LRRC8A expression. It will be appreciated that a molecule having an indirect effect on LRRC8A expression can, per se, be a positive (i.e. activating) or negative (i.e. inhibiting) regulator of its target molecule, as long as the overall effect on LRRC8A is that of activation of LRRC8A.

Compounds of class (ii) increase the biological activity of the protein to be activated. Biological activity denotes in particular any known function of LRRC8A including functions elucidated in accordance with the present invention. Non-limiting examples of said function include its VRAC activity as e.g. described in Example 5 below as well as its activity as a differentiation regulator of keratinocytes, as e.g. described in Example 6 below. In addition, it has been shown that LRRC8A interacts with the PI3K/AKT pathway in lymphocytes via a GRB2-GAB2 complex and the lymphocyte specific receptor tyrosine kinase (LCK). The constitutive association of LRRC8A with the GRB2-GAB2-LCK complex activates AKT via LCK-ZAP-70-GAB2-PI3K, whereas in the absence of LRRC8A, the activation of AKT decreases¹⁹. LRRC8A also plays a role in adipocytes, where an increase in adipocyte size is linked with an increase in insulin signaling by titrating the activity of the insulin-PI3K-AKT2-GLUT4 signaling pathway via LRRC8A. In detail, the complex of insulin receptor (IR) linked to GRB2 and insulin receptor substrate (IRS) acts as a negative regulator. Co-immunoprecipitation experiments have shown that LRRC8A is also residing in this insulin signaling complex and that this interaction of LRRC8A with GRB2 is mediated by the C-terminal leucine rich repeat domain (LRR) of LRRC8A²⁰. Binding of LRRC8A via its LRR domain to GRB2 disrupts the negative inhibition of insulin receptor signaling. Thus, LRRC8A has been shown to also function as an important component of various PI3K pathways in different cell types via the interaction of its LRR domain with GRB2. All these functions of LRRC8A can be tested for by the skilled person either on the basis of common general knowledge or on the basis of the teachings of this specification, optionally in conjunction with the teachings of the documents cited herein.

Also compounds of class (ii) include compounds that have a directly activating effect on LRRC8A but also molecules that are indirectly activating, e.g. by interacting for example with molecules that regulate LRRC8A activity. Again, it will be appreciated that a molecule having an indirect effect on LRRC8A can be a positive (i.e. activating) or negative (i.e. inhibiting) regulator, as long as the overall effect on LRRC8A is an activation. As a non-limiting example, two recent studies suggested that an intracellular pH change as well as mechanical membrane stretching, which is sensed by angiotensin II AT1 receptors AT1R, leads to activation of the NADPH oxidase (NOX) enzyme complex, which then leads to activation of LRRC8A^{21,22}. Thus, the results from these two studies suggest that LRRC8A mediated VRAC activity can be activated by compounds that activate the NOX complex, while NOX inhibitors would result in a decrease in LRRC8A-mediated VRAC activity.

In accordance with the present invention, it is preferred that the activator acts directly on LRRC8A, more preferably it directly increases the transcription and/or translation of LRRC8A.

Both naturally occurring as well as artificial transcriptional regulators of the LRRC8A gene and the LRRC8A genomic locus can be employed as activators of LRRC8A in accordance with the present invention, as well as naturally occurring or artificial regulators of the LRRC8A protein activity. Stimulation or overexpression of said regulators of the LRRC8A gene, the LRRC8A genomic locus, or the LRRC8A protein activity represents a suitable means in order to activate the expression and/or activity of LRRC8A. Preferably, the activator is provided as a nucleic acid molecule, as a small molecule, or as a proteinaceous compound, such as e.g. an antibody or an antibody mimetic or peptide aptamer.

Activators provided as nucleic acid molecules can, for example, be activators that are encoded by a nucleic acid molecule, which can e.g. be incorporated into an expression vector comprising regulatory elements, such as keratinocyte-specific promoters. The activator can also be provided as an activating nucleic acid molecule in form of e.g. programmable sequence-specific genome editing tools such as Zinc-finger nucleases (ZNFs) and transcriptional activator-like effector nucleases (TALENs), as well as CRISPR-Cas9- and CRISPR-Cpf1-based methods, as described e.g. in Wang et al.²³.

CRISPR/Cas9, as well as CRISPR-Cpf1, technologies are applicable in nearly all model organisms and can be used for knock out mutations, chromosomal deletions, editing of DNA sequences and regulation of gene expression. The regulation of the gene expression can be manipulated by the use of a catalytically dead Cas9 enzyme (dCas9) that is conjugated with a transcriptional repressor to repress transcription or with a transcriptional activator for activation of transcription of a specific gene. Similarly, catalytically inactive, “dead” Cpf1 nuclease (CRISPR from Prevotella and Francisella-1) can be fused to synthetic transcriptional repressors or activators to down- or upregulate endogenous promoters²⁴. The exemplary approaches described in the following can, thus, also be carried out with Cpf1 instead of dCas9.

For the activation of gene transcription, e.g. of LRRC8A gene expression, dCas9 is genetically fused with the C-terminal VP64 trans-activation domain. To further improve the potency of dCas9-VP64-mediated gene activation, an advanced system has been developed. For that system, Konermann et al.²⁵ engineered the single-guide RNA (sgRNA), which directs the Cas9 to defined regions in the genome. Two hairpin aptamers were appended, which selectively bind dimerized MS2 bacteriophage coat proteins. In addition, MS2 proteins are fused to p65 and HSF1 transactivation domains, which together form a MS2-p65-HSF1 complex. Taken together, the MS2-p65-HSF1 fusion proteins bind to the hair pin aptamer of the sgRNA, which in turn is incorporated in the dCas9-VP64 fusion protein and forms the final dCas9-SAM complex. The dCsSAM complex gets recruited to the target gene promoter via the designed sgRNA and enhances the recruitment of multiple transcription factors around the promotor, which finally leads to increased expression of the target gene²⁵.

By designing sgRNAs that specifically target the regulatory DNA region upstream of the transcriptional start site of LRRC8A, such as e.g. the LRRC8A promoter, it is thus possible to recruit the dCas9-SAM complex to specific regulatory DNA regions of psoriatic keratinocytes, thereby leading to the activation of LRRC8A gene expression. Two transcriptional start sites of LRRC8A are presently annotated, which lead to the formation of three different LRRC8A mRNA variants (NM_019594 (SEQ ID NO:3), NM_001127244 (SEQ ID NO:4), NM_001127245; SEQ ID NO:5), which, however, all lead to the formation of the same LRRC8A protein. Since not only one but two transcriptional start sites are mapped, two regulatory DNA sequences that lie 3500 bp upstream of the transcriptional start sites can also be defined and relied on for designing sgRNAs that recruit the dCas9-SAM complex. These regulatory sequences, provided herein as SEQ ID NO: 6 and SEQ ID NO:7, contain 3500 bp upstream of the transcriptional start site, as well as the first nucleotide coding for the mRNA transcript.

Alternatively, the DNA-binding domain of zinc finger nucleases (ZFNs) or transcription activator-like effector nucleases (TALENs) can be designed to specifically recognize the LRRC8A promoter region or its 5′-UTR. Fusion constructs of such DNA-binding domains with transcriptional activator domains, such as those described above with regard to CRISPR/Cas9 and CRISPR_Cpf1 can, thus, also be employed to enhance LRRC8A gene expression in psoriatic keratinocytes.

Activators provided as inhibiting nucleic acid molecules that target a regulatory molecule involved in LRRC8A expression are also envisaged herein. Such molecules, which reduce or abolish the expression of a regulatory molecule include, without being limiting, meganucleases, zinc finger nucleases and transcription activator-like (TAL) effector (TALE) nucleases. Such methods are described e.g. in Silva, G et al., 2011, Miller, J C et al. 2011 or Klug, A. 2010²⁶⁻²⁸.

A “small molecule” according to the present invention may be, for example, an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small molecule has a molecular weight of less than about 2000 amu, or less than about 1000 amu such as less than about 500 amu, and even more preferably less than about 250 amu. The size of a small molecule can be determined by methods well-known in the art, e.g., mass spectrometry. The small molecules may be designed, for example, based on the crystal structure of the target molecule, where sites presumably responsible for the biological activity, can be identified and verified in in vivo assays such as in vivo high-throughput screening (HTS) assays.

The term “antibody” as used in accordance with the present invention comprises polyclonal and monoclonal antibodies, as well as derivatives or fragments thereof, which still retain binding specificity. Antibody fragments or derivatives comprise, inter alia, Fab or Fab′ fragments as well as Fd, F(ab′)₂, Fv or scFv fragments; see for example Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999. The term “antibody” also includes embodiments such as chimeric (human constant domain, non-human variable domain), single chain and humanized (human antibody with the exception of non-human CDRs) antibodies.

Various techniques for the production of antibodies are well known in the art and described, e.g. in Harlow and Lane (1988) and (1999), loc. cit. In addition, the antibodies can be produced as peptidomimetics. Further, techniques described for the production of single chain antibodies (see, inter alia, U.S. Pat. No. 4,946,778) can be adapted to produce single chain antibodies specific for the target of this invention. Also, transgenic animals or plants (see, e.g., U.S. Pat. No. 6,080,560) may be used to express (humanized) antibodies specific for the target of this invention. Most preferably, the antibody is a monoclonal antibody, such as a human or humanized antibody. For the preparation of monoclonal antibodies, any technique which provides antibodies produced by continuous cell line cultures can be used. Examples for such techniques are described, e.g. in Harlow and Lane (1988) and (1999), loc. cit. and include the hybridoma technique originally developed by Köhler and Milstein Nature 256 (1975), 495-497, the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Surface plasmon resonance as employed in the BIAcore system can be used to increase the efficiency of phage antibodies which bind to an epitope of the target protein (Schier, Human Antibodies Hybridomas 7 (1996), 97-105; Malmborg, J. Immunol. Methods 183 (1995), 7-13). It is also envisaged in the context of this invention that the term “antibody” comprises antibody constructs which may be expressed in cells, e.g. antibody constructs which may be transfected and/or transduced via, inter alia, viruses or plasmid vectors.

As used herein, the term “antibody mimetics” refers to compounds which, like antibodies, can specifically bind antigens, but which are not structurally related to antibodies. Antibody mimetics are usually artificial peptides or proteins with a molar mass of about 3 to 20 kDa. For example, an antibody mimetic may be selected from the group consisting of affibodies, adnectins, anticalins, DARPins, avimers, nanofitins, affilins, Kunitz domain peptides and Fynomers®. These polypeptides are well known in the art and are described briefly herein below.

The term “affibody”, as used herein, refers to a family of antibody mimetics which is derived from the Z-domain of staphylococcal protein A. Structurally, affibody molecules are based on a three-helix bundle domain which can also be incorporated into fusion proteins. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “adnectin” (also referred to as “monobody”), as used herein, relates to a molecule based on the 10^{th} extracellular domain of human fibronectin Ill (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity can be genetically engineered by introducing modifications in specific loops of the protein.

The term “anticalin”, as used herein, refers to an engineered protein derived from a lipocalin (Beste G, Schmidt F S, Stibora T, Skerra A. (1999) Proc Natl Acad Sci USA. 96(5):1898-903; Gebauer and Skerra (2009) Curr Opinion in Chemical Biology 13:245-255). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

As used herein, the term “DARPin” refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “avimer”, as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity can be selected, for example, by phage display techniques. The binding specificity of the different A-domains contained in an avimer may, but does not have to be identical (Weidle U H, et al., (2013), Cancer Genomics Proteomics; 10(4):155-68).

A “nanofitin” (also known as affitin) is an antibody mimetic protein that is derived from the DNA binding protein Sac7d of Sulfolobus acidocaldarius. Nanofitins usually have a molecular weight of around 7 kDa and are designed to specifically bind a target molecule by randomising the amino acids on the binding surface (Mouratou B, Behar G, Paillard-Laurance L, Colinet S, Pecorari F., (2012) Methods Mol Biol.; 805:315-31).

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity is effected, for example, by phage display or ribosome display techniques. Depending on the scaffold, affilins have a molecular weight of approximately 10 or 20 kDa. As used herein, the term affilin also refers to di- or multimerised forms of affilins (Weidle U H, et al., (2013), Cancer Genomics Proteomics; 10(4):155-68).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDA and domains with the required target specificity can be selected by display techniques such as phage display (Weidle et al., (2013), Cancer Genomics Proteomics; 10(4):155-68).

As used herein, the term “Fynomer®” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

Another example of proteinaceous compounds are peptide aptamers. Aptamers per se are nucleic acid molecules or peptide molecules that bind a specific target molecule. Aptamers are usually created by selecting them from a large random sequence pool, but natural aptamers also exist in riboswitches. Aptamers can be combined with ribozymes to self-cleave in the presence of their target molecule (Osborne et. al. (1997), Current Opinion in Chemical Biology, 1:5-9; Stull & Szoka (1995), Pharmaceutical Research, 12, 4:465-483).

Aptamers offer the utility for biotechnological and therapeutic applications as they offer molecular recognition properties that rival those of the commonly used biomolecules, in particular antibodies. In addition to their discriminatory recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamers' inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

The term “peptide” as used herein describes a group of molecules consisting of up to 30 amino acids, whereas the term “polypeptide” (also referred to as “protein”) as used herein describes a group of molecules consisting of more than 30 amino acids. The group of peptides and polypeptides are referred to together by using the term “(poly)peptide”.

Activators provided as nucleic acid molecules further include nucleic acid aptamers, siRNA, shRNA, miRNA, ribozymes, or antisense nucleic acid molecules.

Aptamers have been described herein above. Nucleic acid aptamers are nucleic acid species that normally consist of (usually short) strands of oligonucleotides. Typically, they have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics).

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of the respective target after introduction into the respective cells.

A ribozyme (from ribonucleic acid enzyme, also called RNA enzyme or catalytic RNA) is an RNA molecule that catalyses a chemical reaction. Many natural ribozymes catalyse either their own cleavage or the cleavage of other RNAs, but they have also been found to catalyse the aminotransferase activity of the ribosome. Non-limiting examples of well-characterised small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in recent years. The hammerhead ribozymes are characterised best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: A region of interest of the RNA, which contains the GUC (or CUC) triplet, is selected. Two oligonucleotide strands, each usually with 6 to 8 nucleotides, are taken and the catalytic hammerhead sequence is inserted between them. The best results are usually obtained with short ribozymes and target sequences.

A recent development, also useful in accordance with the present invention, is the combination of an aptamer, recognizing a small compound, with a hammerhead ribozyme. The conformational change induced in the aptamer upon binding the target molecule can regulate the catalytic function of the ribozyme.

The term “antisense nucleic acid molecule”, as used herein, refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

The function of any of the activators referred to in the present invention may be identified and/or verified by using high throughput screening assays (HTS). Preferably, the level of activity is at least 10% higher than the activity in the absence of the activator; more preferably, the level of activity is at least 20% higher, such as at least 30% higher, more preferably at least 40% higher than the activity in the absence of the activator. Yet more preferred are activators enhancing the level of activity to at least 50%, at least 100%, at least 200% or at least 500% higher than the activity in the absence of the activator.

The efficiency of the activator can be quantified by comparing the level of activity in the presence of the activator to that in the absence of the activator. For example, as an activity measure may be used: a change in amount of mRNA formed, a change in amount of protein formed, a change in activity of volume-regulated anion channels (VRACs) comprising LRRC8A, a change in activity of PI3K-Akt-signalling and/or a change in the cellular phenotype or in the phenotype of an organism, for example based on the expression of specific marker genes as detailed further below.

Means and methods to determine the amount of LRRC8A expression in a sample can be carried out on the nucleic acid level or on the amino acid level. Methods for determining the expression of a protein on the nucleic acid level include, but are not limited to, northern blotting, PCR, RT-PCR or real time RT-PCR, microarray analysis and RNA sequencing, all of which are well known in the art, as for example in Molecular Biology of the cell, 5th edition, 2007, Chapter 8, Garland Science, by Bruce Alberts et al. Methods for the determination of the expression of a protein on the amino acid level, which are also well known in the art (e.g. Alberts et al. Molecular Biology of the cell, 5th edition, 2007, Chapter 8, Garland Science), include but are not limited to western blotting or polyacrylamide gel electrophoresis in conjunction with protein staining techniques such as Coomassie Brilliant blue, silver-staining, as well as antibody staining.

Means and methods to determine a change in activity of volume-regulated anion channels (VRACs) comprising LRRC8A include, without being limiting, measuring iodide influx, measuring ion currents, measuring the release of VRAC substrates, measuring cell volume changes, measuring cell stiffness, impedance measurements, cell size determination, or electrical sensing zone method. These methods have been described in the art, e.g. in ^{18,29-31} and are discussed in more detail herein below.

In accordance with the present invention, the LRRC8A and/or the activator of LRRC8A is for use in the treatment and/or prevention of a skin condition associated with an altered differentiation of keratinocytes. Preferably, the LRRC8A and/or the activator of LRRC8A is for use in the treatment and/or prevention of a skin condition in humans.

As mentioned, the human skin is composed of several layers of differentiating keratinocytes. The homeostasis of the healthy epidermis is maintained by a tightly regulated balance between keratinocyte proliferation and differentiation. Disturbance of this balance, for example in form of an altered differentiation of keratinocytes, leads to skin disorders such as psoriasis and atopic dermatitis. In accordance with the present invention, the term “altered differentiation of keratinocytes” relates to a disturbed differentiation pattern, i.e. a pattern that is not observed in the healthy skin. Whether the differentiation of keratinocytes is altered can be determined by the skilled person without further ado, for example by comparing the differentiation with that of keratinocytes known to be from a sample of healthy skin, or by comparing the differentiation pattern with published data on differentiation in healthy skin or with the data relating to healthy skin provided herein below in the appended examples.

In an alternative embodiment, the present invention relates to a method of treatment and/or prevention of a skin condition associated with an altered differentiation of keratinocytes, the method comprising administering the leucine-rich repeat-containing protein 8A (LRRC8A), and/or an activator of LRRC8A to a subject in need thereof. All definition and preferred embodiments provided herein with regard to the LRRC8A, and/or an activator of LRRC8A, for use in accordance with the invention apply mutatis mutandis to this method of treatment.

In accordance with the present invention, it was surprisingly found that the leucine-rich repeat-containing protein 8A (LRRC8A) plays an important role in the differentiation of keratinocytes.

LRRC8A was the first component of volume-regulated anion channels (VRACs) that has been identified and it represents an essential component of VRACs with biophysical and pharmacological properties of native VRACs^{18,31} VRACs play an important role in a process called “regulatory volume decrease (RVD)”, a process by which cells restore their initial cell volume in response to osmotic stress. Via an increase of extracellular osmolytes an osmotic gradient is established which provides the driving force to move water back into the extracellular space, thereby leading to a cell volume decrease³²⁻³⁴.

Cell volume regulation is an integral part of many physiological processes such as apoptosis, trans-epithelial ion transport, migration and proliferation^{32,35,36}. However, the role of regulated volume changes during the differentiation of keratinocytes has not been elucidated in much detail yet. Despite the fact that keratinocytes undergo obvious morphological and cell size changes when they move from the basal to the granular level³⁷, it is not known whether this involves any actively controlled regulatory mechanisms such as RVD and activation of VRACs. Some, but interestingly not all, studies suggest that hypotonic and hypertonic stress can differentially affect proliferation of HaCaT cells³⁸⁻⁴⁰, which is also accompanied with gene expression changes of differentiation markers such as involucrin, fillagrin and transglutaminase^{38,39}.

Also the function of the VRAC component LRRC8A in keratinocytes has not been studied in the art. So far, it was not known whether LRRC8A is a major component of VRACs and nothing was known with regard to the role of LRRC8A in physiological processes such as RVD and keratinocyte differentiation. In accordance with the present invention, it is shown for the first time that LRRC8A is a major component of VRACs and that it mediates part of RVD in keratinocytes. It is further unexpectedly shown that LRRC8A plays a significant role in normal as well as in aberrant, pathological keratinocyte differentiation. In particular, it was shown that LRRC8A expression is downregulated in the epidermis of psoriasis patients. During terminal differentiation of healthy skin, basal keratinocytes develop into corneal keratinocytes. This process is based on tightly controlled changes between proliferation and differentiation. In basal keratinocytes, proliferation is high whereas differentiation is low. When keratinocytes further develop into the different keratinocyte layers, proliferation decreases, whereas differentiation further progresses. During this process, LRRC8A is not constant; its protein level changes: it is found that LRRC8A is preferentially localized in basal layers and declines towards the outer, more differentiated keratinocyte layers in the human skin. Further it is shown that LRRC8A expression is dynamically regulated and dependent on the differentiation stage: LRRC8A first increases until it reaches its maximum and then declines at the latest stage of differentiation. This dynamic change of LRRC8A expression is characteristic for healthy keratinocyte development.

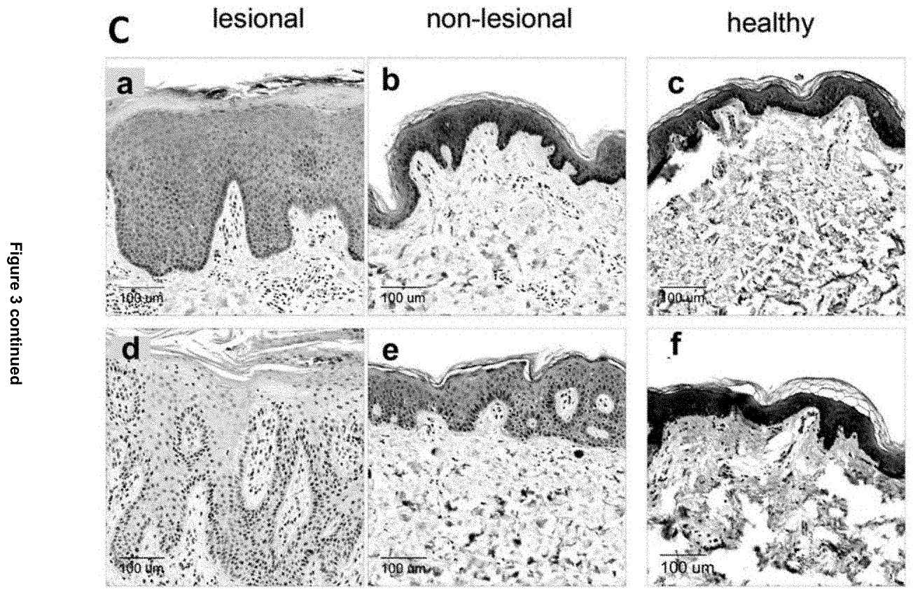

In diseased skin, such as in psoriasis, the equilibrium between proliferation and differentiation is disturbed. Differentiation is abnormal and slower, whereas proliferation is faster, which finally leads to abnormally formed keratinocyte layers and to the diseased skin condition. It is found herein that during terminal differentiation of keratinocytes in psoriasis, LRRC8A expression is also abnormally changed. First, in basal stages, LRRC8A expression is reduced and does not increase as fast as in healthy skin. At later stages, LRRC8A levels stay higher and do not decrease as compared to healthy skin. Furthermore, it is found herein that in lesional skin of psoriasis patients, LRRC8A expression is reduced, as shown in FIG. 3C. Accordingly, the LRRC8A expression pattern is disturbed in psoriatic keratinocytes, thereby suggesting that LRRC8A is important for ordered keratinocyte differentiation.

Importantly, as shown in Example 6, modulation of LRRC8A activity via reducing LRRC8A gene dosage enables the manipulation of this differentiation process. When LRRC8A levels were reduced, keratinocytes started to develop abnormally. Their gene expression pattern surprisingly showed striking similarities to psoriasis, leading to the conclusion that LRRC8A is required for normal differentiation. More specifically, it is found that at early stages of differentiation, when LRRC8A levels are too low, e.g. in case of psoriasis, or when LRRC8A are completely absent, e.g. in case of LRRC8A knock-out, cells were not able to undergo their normal differentiation program.

These findings suggest that for the treatment of skin conditions such as psoriasis, LRRC8A activity/expression levels have to be restored to normal levels, i.e. by increasing LRRC8A activity/expression. This finding is surprising as in transcriptome-wide studies^{5,7,41-43} LRRC8A levels in psoriatic skin were described to be higher compared to healthy skin. Based on these observations, one would have concluded that a suitable treatment of psoriasis requires a reduction—and not an increase—in LRRC8A level/activity. In accordance with the present invention it was found that such a conclusion would not have been valid. The finding of an overall higher total LRRC8A level in psoriatic skin in these transcriptome-wide studies can be explained by the fact that total gene expression levels in whole skin were measured. Importantly, however, this approach overlooked the dynamic changes in LRRC8A levels that occur during differentiation and that were discovered here for the first time. In other words, the findings of these transcriptome-wide studies ignore that LRRC8A levels are initially lower in the first stages of differentiation, i.e. at the basal state, in psoriatic compared to healthy keratinocytes and also increase too slowly in psoriatic compared to healthy keratinocytes. It is only at the latest stages, i.e. the corneal layer, that the level of LRRC8A is higher in psoriatic keratinocytes as compared to healthy keratinocytes.

In addition, the findings of these transcriptome-wide studies are further misleading as they only measured LRRC8A mRNA levels and not the actual amount of LRRC8A protein that is formed. In examples below, on the other hand, LRRC8A protein was directly detected by immunohistological analysis using LRRC8A antibody and it was clearly shown that LRRC8A protein levels are reduced in lesional skin of psoriasis patients.

Thus, it was surprisingly found in accordance with the present invention that treatment of psoriasis does not require decreasing but increasing LRRC8A levels/activity in order to restore the normal LRRC8A levels in the basal and proceeding keratinocytes. Basal and further matured keratinocytes with almost normal LRRC8A level can then start to develop their normal terminal differentiation program leading to less psoriatic keratinocyte layers.

In accordance with the present invention, the LRRC8A and/or the activator can be used in combination with one or more additional compounds selected from LRRC8B and/or an activator of LRRC8B; LRRC8C and/or an activator of LRRC8C; LRRC8D and/or an activator of LRRC8D; LRRC8E and/or an activator of LRRC8E; and LRRC8F and/or an activator of LRRC8F.

Depending on the build-up of the specific LRRC8A-containing VRAC to be targeted, the skilled person can make the appropriate choice of an additional compound being a different subtype of LRRC8 or an additional compound being targeted to a different subtype of LRRC8, without further ado. Thus, if the VRAC, for example, is known to consist of LRRC8A and LRRC8B, then either LRRC8B or an activator of LRRC8B (or both) can be chosen as an additional compound in accordance with this preferred embodiment.

More preferably, the LRRC8A and/or the activator in accordance with the present invention is the only active compound that targets LRRC8. In other words, in this preferred embodiment, LRRC8A and/or the activator of LRRC8A is used without the additional use of a compound selected from LRRC8B and/or an activator of LRRC8B; LRRC8C and/or an activator of LRRC8C; LRRC8D and/or an activator of LRRC8D; LRRC8E and/or an activator of LRRC8E; and LRRC8F and/or an activator of LRRC8F.

In a preferred embodiment of the LRRC8A and/or the activator for use according to the invention, the skin condition associated with an altered differentiation of keratinocytes is a condition characterised by enhanced epidermal proliferation.

As detailed herein above, the homeostasis of the healthy epidermis relies on a tightly regulated balance between keratinocyte proliferation and differentiation. In accordance with the preferred embodiment, the skin condition is not only characterized by changes in the differentiation of keratinocytes, but also by an enhanced epidermal proliferation. More preferably, the skin condition is a condition characterised by enhanced proliferation of keratinocytes.

The term “enhanced proliferation” as used herein, relates to an increased rate of cell growth and division as compared to the rate of cell growth and division observed in healthy cells. Whether proliferation is enhanced can be determined by the skilled person without further ado, e.g. by comparing affected and unaffected skin samples or by comparing the rate of proliferation with published or pre-determined data for healthy samples.

In a further preferred embodiment of the LRRC8A and/or the activator for use according to the invention, the skin condition associated with an altered differentiation of keratinocytes is psoriasis or dermatitis, preferably atopic dermatitis.

All of the skin conditions described herein are well known to the skilled person and are defined in accordance with the prior art and the common general knowledge of the skilled person.

The term “psoriasis”, as used herein, includes all of the five main types of psoriasis, namely plaque psoriasis, guttate psoriasis, inverse psoriasis, pustular psoriasis, and erythrodermic psoriasis. Histologically, psoriasis is characterized by a thickened, irregular stratum corneum with parakeratosis, epidermal thickening with acanthosis and an absence of the granular layer. This is caused by hyperproliferating keratinocytes that are unable to properly initiate the epidermal differentiation program⁴⁴, which results in the delocalization of the differentiation marker involucrin (IVL) into the spinous and granular layer, as well as reduced expression of the differentiation markers keratins (KRT) and filaggrin (FLG). In addition more Ki-67 positive nuclei can be detected in the basal layer, indicating proliferation.

The term “dermatitis” is a medical term used to describe any type of skin inflammation. There are various subtypes of dermatitis, which are often characterized by dry, irritated skin. The term “eczema” relates to those subtypes of dermatitis that are characterized by itchy skin as an additional symptom. Generally, in the affected skin, the barrier function is impaired making the skin susceptible to allergens and environmental stressors. As a consequence this can lead to skin sensitization and inflammation⁴⁵. The term “dermatitis”, as used herein, includes atopic dermatitis, allergic contact dermatitis, irritant contact dermatitis, and stasis dermatitis, with atopic dermatitis (AD) being the most common form that usually begins in childhood.

AD is a form of eczema that is characterized by itchy, red, swollen, and cracked skin. A skin biopsy taken from a site with acute atopic dermatitis is characterized by intercellular oedema, perivascular infiltrates primarily of lymphocytes, and retention of the nuclei of the keratinocytes as they ascend into the stratum corneum, so-called parakeratosis. Chronic sites are dominated by a thickened stratum corneum, so-called hyperkeratosis, a thickened stratum spinosum (acanthosis), but sparse lymphocytic infiltrates⁴⁶.

In an even more preferred embodiment of the activator of LRRC8A for use according to the invention, the activator increases the expression of LRRC8A and/or the activity of volume-regulated anion channels (VRACs) comprising LRRC8A.

According to this preferred embodiment, the activator increases the expression of LRRC8A and/or the activity of volume-regulated anion channels (VRACs) comprising LRRC8A. An activator is considered to increase the expression of LRRC8A if the amount of LRRC8A is higher in the presence of the activator than in the absence of the activator. Preferably, the amount of LRRC8A is at least 10% higher in the presence of the activator than in the absence of the activator; more preferably, the amount of LRRC8A is at least 20%, such as at least 30%, more preferably at least 40% higher in the presence of the activator than in the absence of the activator. Yet more preferably, the amount of LRRC8A is at least 50%, at least 100%, at least 200% or at least 500% higher in the presence of the activator than in the absence of the activator. With “amount of LRRC8A”, as used with regard to this embodiment, the amount of LRRC8A mRNA or protein is referred to, and preferably the amount of LRRC8A protein.

Means and methods for determining the amount of expression of LRRC8A have been provided herein above.

The activator can further increase the activity of VRACs comprising LRRC8A. It will be appreciated that this activator is limited to an “activator of LRRC8A”, i.e. its action on increasing the activity of VRACs comprising LRRC8A necessarily is via an activation of LRRC8A.

An activator is considered to increase the activity of VRACs comprising LRRC8A, if the activity of VRACs comprising LRRC8A is higher in the presence of the activator than in the absence of the activator. Preferably, the activity of VRACs comprising LRRC8A is at least 10% higher in the presence of the activator than in the absence of the activator; more preferably, the activity of VRACs comprising LRRC8A is at least 20% higher, such as at least 30% higher, more preferably at least 40% higher in the presence of the activator than in the absence of the activator. Yet more preferably, the activity of VRACs comprising LRRC8A is at least 50%, at least 100%, at least 200% or at least 500% higher in the presence of the activator than in the absence of the activator.

In accordance with this preferred embodiment, an activator can either act on the expression of LRRC8A, or on the activity of LRRC8A with regard to its role as a component of VRACs, or both.

Thus, increasing the expression of LRRC8A is one means by which the activator of the invention can act. Based on the data provided herein with regard to LRRC8A knock-down (see Example 6), it is stipulated that the presence of increased amounts of LRRC8A leads to a correction in the aberrant differentiation pattern observed in the recited skin disorders.

Alternatively, or additionally, the activator can increase the activity of the LRRC8A protein. As discussed herein above, LRRC8A has recently been identified as an essential component of volume-regulated anion channels, i.e. VRACs, with biophysical and pharmacological properties of native VRACs^{18,31}. Cell volume regulation is an integral part of many physiological processes such as apoptosis, transepithelial ion transport, migration and proliferation^{32,35,36}. However, the role of regulated volume changes during the differentiation of keratinocytes has not been elucidated in much detail yet. Despite the fact that keratinocytes undergo obvious morphological and cell size changes when they move from the basal to the granular level³⁷, it is not known whether this involves any actively controlled regulatory mechanisms such as RVD and activation of VRACs. Some, but interestingly not all, studies suggest that hypotonic and hypertonic stress can differentially affect proliferation of HaCaT cells³⁸⁻⁴⁰ which is also accompanied with gene expression changes of differentiation markers such as involucrin, fillagrin and transglutaminase^{38,39}.

As is shown in the data provided herein (see Example 5), LRRC8A knock-down in HaCaT cells led to a decrease in VRAC activity, in particular, the regulatory volume decrease (RVD) was drastically reduced in LRRC8A knock-out cells compared to HaCaT wildtype cells. Based on these observations, it is stipulated that an increase of LRRC8A protein activity leads to a higher activity of VRACs comprising LRRC8A.

In another preferred embodiment of the activator of LRRC8A for use in accordance with the present invention, the activator is
(i) a vector encoding, in expressible form, LRRC8A; or
(ii) a regulator of gene expression that up-regulates the expression of endogenously present LRRC8A.

According to option (i), the activator is a vector encoding LRRC8A in expressible form, i.e. in a form that enables the expression of the LRRC8A protein encoded by a corresponding nucleic acid molecule. Expression of a nucleic acid molecule can for example be ensured by employing regulatory elements. Regulatory elements/sequences are well known to those skilled in the art and include, without being limiting, regulatory sequences ensuring the initiation of transcription, internal ribosomal entry sites (IRES) (Owens, Proc. Natl. Acad. Sci. USA 98 (2001), 1471-1476) and optionally regulatory elements ensuring termination of transcription and stabilisation of the transcript.

Non-limiting examples for regulatory elements ensuring the initiation of transcription comprise a translation initiation codon, enhancers such as e.g. the SV40-enhancer, insulators and/or promoters, such as for example the cytomegalovirus (CMV) promoter, SV40-promoter, RSV (Rous sarcoma virus)-promoter, the lacZ promoter, chicken beta-actin promoter, CAG-promoter (a combination of chicken beta-actin promoter and cytomegalovirus immediate-early enhancer), the gai10 promoter, human elongation factor 1α-promoter, AOX1 promoter, GAL1 promoter CaM-kinase promoter, the lac, trp or tac promoter, the lacUV5 promoter, the Autographa californica multiple nuclear polyhedrosis virus (AcMNPV) polyhedral promoter or a globin intron.

Non-limiting examples for regulatory elements ensuring transcription termination include the V40-poly-A site, the tk-poly-A site or the SV40, lacZ or AcMNPV polyhedral polyadenylation signals, which are to be included downstream of the nucleic acid sequence to be expressed. Additional regulatory elements may include translational enhancers, Kozak sequences and intervening sequences flanked by donor and acceptor sites for RNA splicing, nucleotide sequences encoding secretion signals or, depending on the expression system used, signal sequences capable of directing the expressed polypeptide to a cellular compartment. Moreover, elements such as origin of replication, drug resistance genes, regulators (as part of an inducible promoter) may also be included.

Furthermore, additional sequences such as e.g. selectable markers may be introduced together with the nucleic acid molecule encoding LRRC8A. The co-transfection with a selectable marker such as dhfr, gpt, G418, neomycin, hygromycin allows the identification and isolation of the transfected cells. The dhfr (dihydrofolate reductase) marker is useful to develop cell lines that carry several hundred or even several thousand copies of the gene of interest. Another useful selection marker is the enzyme glutamine synthase (GS). Using these markers, the cells are grown in selective medium and the cells with the highest resistance are selected.

In accordance with this preferred embodiment, the nucleic acid molecule encoding LRRC8A as well as potential regulatory sequences and additional sequences are comprised in an expression vector. Preferably, the vector is a plasmid, cosmid, virus, bacteriophage or another vector used conventionally e.g. in genetic engineering. Non-limiting examples include prokaryotic plasmid vectors, such as the pET-series of expression vectors (Novagen), the pUC-series, pBluescript (Stratagene) or pCRTOPO (Invitrogen), lambda gt11, pJOE, the pBBR1-MCS series, pJB861, pBSMuL, pBC2, pUCPKS, pTACT1 and vectors compatible with expression in mammalian cells like E-027 pCAG Kosak-Cherry (L45a) vector system, pREP (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, plZD35, Okayama-Berg cDNA expression vector pcDV1 (Pharmacia), pRc/CMV, pcDNA1, pcDNA3 (Invitrogen), pSPORT1 (GIBCO BRL), pGEMHE (Promega), pLXIN, pSIR (Clontech), pIRES-EGFP (Clontech), pEAK-10 (Edge Biosystems) pTriEx-Hygro (Novagen) and pCINeo (Promega).

The coding sequences inserted into the vector can be synthesized by standard methods. Ligation of the coding sequences to transcriptional regulatory elements can be carried out using established methods. For vector modification techniques, see Sambrook and Russel, 2001. As a non-limiting example, the nucleic acid sequence provided herein as SEQ ID NO:1 can be employed for insertion into a vector and expression of LRRC8A. As a further example, any of the nucleic acid sequences provided herein as SEQ ID NOs:3 to 5 may also be employed.

Alternatively, the activator can also be a regulator of gene expression that up-regulates the expression of endogenously present LRRC8A. Such a regulator can be a naturally occurring regulator that is already present in the cell and that normally regulates expression of LRRC8A. In addition, or alternatively, regulators can be employed that are not normally present in keratinocytes and/or that do not normally act as LRRC8A regulators in keratinocytes. Such regulators of gene expression that up-regulates the expression of endogenously present LRRC8A include, without being limiting, (i) CRISPR-Cas9-based regulators; (ii) CRISPR-Cpf1-based regulators; (iii) programmable sequence-specific genome editing nucleases selected from zinc-finger nucleases (ZNFs) and transcriptional activator-like effector nucleases (TALENs); (iv) meganucleases; (v) small molecules; (vi) antibodies or antibody mimetics; (vii) aptamers; and (viii) inhibitory nucleic acid molecules selected from siRNA, shRNA, miRNA, ribozymes and antisense nucleic acid molecules. Preferably, the regulator is selected from CRISPR-Cas9- and CRISPR-Cpf1-based regulators and programmable sequence-specific genome editing nucleases, such as e.g. zinc-finger nucleases (ZNFs), or transcriptional activator-like effector nucleases (TALENs). Details with regard to these regulators have been provided herein above.

The present invention further relates to a method of identifying a compound capable of altering the differentiation of keratinocytes, the method comprising the steps of (a) contacting keratinocytes with a test compound and determining the amount of LRRC8A protein or LRRC8A transcript in said keratinocytes; and (b) comparing the amount of LRRC8A protein or LRRC8A transcript determined in step (a) with the amount of LRRC8A protein or LRRC8A transcript in a control not contacted with said test compound, wherein a change in the amount of LRRC8A protein or LRRC8A transcript after contacting the keratinocytes with the test compound indicates that the test compound is capable of altering the differentiation of keratinocytes.

In accordance with the first step (a), keratinocytes are contacted with a test compound.

Depending on the type of compound to be identified, the keratinocytes can be healthy cells, i.e. cells that were not obtained from affected tissue of a patient having a skin disorder associated with an altered differentiation of keratinocytes, such as e.g. psoriasis or eczema. Alternatively, the cells can be obtained from affected tissue of a patient having such a skin disorder, or from a cell model reflecting such a skin disorder. For example, various in vitro psoriasis models are known and have been described, e.g. in⁴⁻⁶. Typically, such cell culture models are generated by treating normal (i.e. healthy) human keratinocytes (NHK) with a cocktail of psoriatic cytokines, including e.g. TNF-α and IL-17, thereby inducing the aberrant differentiation of keratinocytes characteristic of psoriasis. These known models can, for example, be used in the method of the present invention.

Healthy cells are of particular relevance in order to obtain basic information on whether a particular compound is capable of altering the differentiation of keratinocytes, whereas diseased cells or cells serving as a model for a disorder associated with an altered differentiation of keratinocytes can be employed in order to investigate said disorder in more detail and to identify potential therapeutically or cosmetically useful compound.

The keratinocytes can be primary cells obtained from an organism of interest or may be an established keratinocyte cell line. Furthermore, the keratinocytes can be cells in individualized form, such as e.g. in a cell culture, or cells comprised within a three-dimensional (3D) structure, such as a tissue biopsy or 3D skin models. Such 3D skin models for psoriasis have being developed recently and are for example commercially available from MatTek Corporation, Ashland, Mass., USA. These models are composed of healthy keratinocytes and diseased fibroblasts isolated from psoriatic lesions of patients. Preferably, such models are amended to include psoriatic epidermal keratinocytes, as it was recently described that the use of such psoriatic epidermal keratinocytes would more closely resemble psoriatic conditions⁴⁷. Since psoriasis involves the complex cross-talk between various cell types and cytokines, 3D skin models that additionally contain immune cells are of great value. The first 3D skin equivalent that achieved to include different T cell populations allowed to study migration of immune cells and secretion of pro-inflammatory cytokines⁴⁸. Such 3D models could also be used to analyze the potential beneficial effects of compounds that act via a modulation of LRRC8A for the treatment of differentiation defects such as psoriasis.

Measurements of LRRC8A protein levels as well as of LRRC8A transcript level can be accomplished in several ways, as described above.

In a second step (step (b)), the thus determined amounts of LRRC8A protein or transcript are compared to the amounts present in a control that was not contacted with the test compound. It will be appreciated that the amount of LRRC8A protein or transcript in a control can be determined prior to carrying out the method of the invention (for example as a step (a-0) prior to step (a)), or in parallel thereto (for example parallel to step (a) or after step (a) and before step (b)). Furthermore, this step can be carried out once to provide a reference value for future use, or may be carried out each time the method is carried out. The amounts determined in step (b) are then compared to this reference value in order to determine whether the amount of LRRC8A protein or transcript has changed upon contacting the keratinocytes with the test compound.

In accordance with the present invention, the finding of a change in the amount of LRRC8A protein or transcript indicates that the test compound is capable of altering the differentiation of keratinocytes. The term “altering”, as used herein with regard to the differentiation of keratinocytes, relates to a change in the rate of differentiation of said cells, both with regard to (i) the overall degree of differentiation, as well as (ii) the time course of differentiation. The term “overall degree of differentiation” relates to the amount of keratinocytes that reach full differentiation. This can be determined, for example, based on the number of undifferentiated keratinocytes that developed into fully differentiated keratinocytes after a specific amount of time. It will be appreciated that differentiation is considered to be altered if the overall degree of differentiation is either increased or decreased.

The term “time course of differentiation”, on the other hand, relates to the speed at which a certain amount of cells reach partial or full differentiation. For example, a defined number of undifferentiated keratinocytes can be treated with the test compound at time point 0, and the differentiation is then assessed after 1 hour, 2 hours, 5 hours, 12 hours and so on. It will be appreciated that the speed at which the keratinocytes differentiate can be increased, it can be decreased, and there can be a combination of both, i.e. an initially change in one direction (either increase or decrease), followed by a change in the other direction. All three aspects are considered to represent an altered differentiation. Furthermore, in healthy epidermal skin cells, differentiation is reciprocally linked to proliferation. Thus, an increased differentiation is typically accompanied by decreased proliferation, while a decreased differentiation is accompanied by an increased proliferation. Accordingly, any change in the rate of differentiation can also be determined, at least in healthy keratinocytes, indirectly via a determination of the proliferation rate of cells.

Said method of the invention can, for example, be accomplished in a high-throughput manner. Robotic equipment for that purpose is known in the art and available from a number of suppliers. Cells are normally grown in wells of plates containing arrays of 96, 384, 1536 or more wells. Transfer of the well-plates from incubators, addition of test compounds, optional washing steps as well determining the read-out is performed in an automated fashion without requiring user interference using hundreds of thousands to millions of compounds in days to weeks.

Once a compound of interest has been identified, it can be further developed into a pharmaceutical or cosmetic agent, such as by reducing its toxicity, prolonging shelf life and so on.

The present invention further relates to a method of identifying a compound capable of altering the differentiation of keratinocytes, the method comprising the steps of (a) contacting keratinocytes with a test compound and determining the activity of (a) VRAC(s) comprising LRRC8A in said keratinocytes; and (b) comparing the activity determined in step (a) with the activity in a control not contacted with said test compound, wherein a change in the activity of (a) VRAC(s) comprising LRRC8A after contacting the keratinocytes with the test compound indicates that the test compound is capable of altering the differentiation of keratinocytes.

The definitions and preferred embodiments provided herein above with regard to the method of the invention based on measurements of the amount of LRRC8A protein or LRRC8A transcript apply mutatis mutandis to this method of the present invention based on measurements of the activity of (a) VRAC(s) comprising LRRC8A.

Means and methods of determining the activity of (a) VRAC(s) comprising LRRC8A are well known in the art^{18,29-31.} Preferably, the activity of (a) VRAC(s) comprising LRRC8A is determined by one or more methods selected from measuring iodide influx, measuring ion currents, measuring the release of VRAC substrates, measuring cell volume changes, measuring cell stiffness, impedance measurements, cell size determination, or an electrical sensing zone method. More preferably, the activity of (a) VRAC(s) comprising LRRC8A is determined by measuring iodide influx using fluorescent halide-sensitive proteins (hsYFP) or ion-sensitive small-molecule dyes, measuring ion currents using electrophysiological methods such as patch-clamp technologies, measuring released VRAC substrates such as neurotransmitters (e.g. GABA), amino acids (e.g. [³H]-D-serine) and radio-labelled osmolytes (such as [³H]-taurine, [¹⁴C]-D-aspartate, myo-inositol), measuring cell volume changes by volume-sensitive fluorescent dyes such as calcein, 3D volume measurements using conventional, confocal or atomic force microscopy, measuring cell stiffness by optical magnetic twisting cytometry (OMTC), impedance measurements, cell size determination by flow cytometry (FACS), or an electrical sensing zone method (Coulter principle).

In a preferred embodiment of the methods of the invention, the method further comprises determining the expression level of at least one marker selected from keratin 1 (KRT1), keratin 10 (KRT10), involucrin (IVL), filaggrin (FLG), loricrin (LOR), keratin 4 (KRT4), keratin 15 (KRT15), transglutaminase 1 (TGM1), S100 calcium binding protein A7 (S100A7), S100 calcium binding protein A8 (S100A8), S100 calcium binding protein A9 (S100A9), C-X-C motif chemokine ligand 1 (CXCL1), C-X-C motif chemokine ligand 8 (CXCL8), small proline rich protein 2C (SPRR2C), small proline rich protein 2D (SPRR2D), serpin family B member 3 (SERPINB3), serpin family B member 4 (SERPINB4), peptidase inhibitor 3 (PI3), lipocalin 2 (LCN2), keratin 6A (KRT6A), keratin 16 (KRT16), beta-defensin 1 (DEFB1) and marker of proliferation Ki-67 (MK167).

All of the markers referred to herein are defined in accordance with the pertinent prior art. The descriptions provided below are based on the information available from: Human gene database “GeneCards”, Weizmann Institute of Science, Version v4.6.1 Build 19, see the World Wide Web at genecards.org/.

Several of the above listed markers, namely keratin 1 (KRT1), keratin 10 (KRT10), keratin 4 (KRT4), keratin 15 (KRT15), keratin 6A (KRT6A) and keratin 16 (KRT16), belong to the keratin gene family. Keratins are heteropolymeric structural proteins which form the intermediate filament. These filaments, along with actin microfilaments and microtubules, compose the cytoskeleton of epithelial cells and are, thus, responsible for the structural integrity of epithelial cells. Keratins are subdivided into cytokeratins and hair keratins. Most of the type I cytokeratins consist of acidic proteins which are arranged in pairs of heterotypic keratin chains. The type II cytokeratins consist of basic or neutral proteins which are arranged in pairs of heterotypic keratin chains co-expressed during differentiation of simple and stratified epithelial tissues.

Keratin 1 (KRT1) is a type II cytokeratin that is encoded in humans by the KRT1 gene. KRT1 is specifically expressed in the spinous and granular layers of the epidermis with family member KRT10. Mutations in the KRT1 and KRT10 genes have been associated with bullous congenital ichthyosiform erythroderma.

Keratin 10 (KRT10) is a type I (acidic) cytokeratin that is encoded in humans by the KRT10 gene. Mutations in the KRT10 gene are associated with epidermolytic hyperkeratosis.

Keratin 4 (KRT4) is a type II cytokeratin that is encoded in humans by the KRT4 gene. KRT4 is specifically expressed in differentiated layers of the mucosal and esophageal epithelia with family member KRT13. Mutations in the KRT4 and KRT13 genes have been associated with white sponge nevus, characterized by oral, esophageal, and anal leukoplakia.

Keratin 15 (KRT15) is a type I cytokeratin that is encoded in humans by the KRT15 gene. Diseases associated with KRT15 include central centrifugal cicatricial alopecia and eccrine sweat gland neoplasm.

Keratin 6A (KRT6A) is a type II cytokeratin that is encoded in humans by the KRT6A gene. So far, six isoforms of human type II keratin-6 (K6) have been identified; the multiplicity of the genes is attributed to successive gene duplication events. KRT6A encodes the most abundant of these isoforms, representing about 77% of all forms found in epithelia. These genes are expressed with family members KRT16 and/or KRT17 in the filiform papillae of the tongue, the stratified epithelial lining of oral mucosa and esophagus, the outer root sheath of hair follicles, and the glandular epithelia. Mutations in the KRT6A, KRT16 and KRT17 genes have been associated with pachyonychia congenita. In addition, peptides from the C-terminal region of KRT6A have antimicrobial activity against bacterial pathogens.

Keratin 16 (KRT16) is a type I cytokeratin that is encoded in humans by the KRT16 gene. KRT16 has been coexpressed with keratin 14 in a number of epithelial tissues, including esophagus, tongue, and hair follicles. Mutations in the KRT16 gene are associated with type 1 pachyonychia congenita, non-epidermolytic palmoplantar keratoderma and unilateral palmoplantar verrucous nevus.

Involucrin (IVL) is a protein that is encoded in humans by the IVL gene. Involucrin, a component of the keratinocyte crosslinked envelope, is found in the cytoplasm and crosslinked to membrane proteins by transglutaminase. Diseases associated with IVL include porokeratosis.

Fillagrin (FLG) is a protein that is encoded in humans by the FLG gene. FLG is an intermediate filament-associated protein that aggregates keratin intermediate filaments in mammalian epidermis. It is initially synthesized as a polyprotein precursor, profilaggrin, which is localized in keratohyalin granules, and is subsequently proteolytically processed into individual functional filaggrin molecules. Mutations in the FLG gene are associated with ichthyosis vulgaris and atopic dermatitis.

Loricrin (LOR) is a protein that is encoded in humans by the LOR gene. Loricrin is a major protein component of the cornified cell envelope found in terminally differentiated epidermal cells. Mutations in the LOR gene are associated with Vohwinkel's syndrome and progressive symmetric erythrokeratoderma, both inherited skin diseases.

The markers S100 calcium binding protein A8 (S100A8), S100 calcium binding protein A9 (S100A9) and S100 calcium binding protein A7 (S100A7) are members of the S100 family of proteins containing 2 EF-hand calcium-binding motifs. S100 proteins are localized in the cytoplasm and/or nucleus of a wide range of cells, and involved in the regulation of a number of cellular processes such as cell cycle progression and differentiation. S100 genes include at least 13 members.

S100A8 is a protein that is encoded in humans by the S100A8 gene. S100A8 is believed to function in the inhibition of casein kinase and as a cytokine. Altered expression of this protein is associated with cystic fibrosis. Multiple transcript variants encoding different isoforms have been found for this gene.

S100A9 is a protein that is encoded in humans by the S100A9 gene. S100A9 is believed to function in the inhibition of casein kinase and altered expression of this protein is associated with cystic fibrosis. This antimicrobial protein exhibits antifungal and antibacterial activity.

S100A7 is a protein that is encoded in humans by the S100A7 gene. S100A7 differs from the other S100 proteins of known structure in its lack of calcium binding ability in one EF-hand at the N-terminus. The protein is overexpressed in hyperproliferative skin diseases, exhibits antimicrobial activities against bacteria and induces immunomodulatory activities.

C-X-C motif chemokine ligand 1 (CXCL1) is a protein that is encoded in humans by the CXCL1 gene. CXCL1 is a member of the CXC subfamily of chemokines. The encoded protein is a secreted growth factor that signals through the G-protein coupled receptor, CXC receptor 2. This protein plays a role in inflammation and as a chemoattractant for neutrophils. Aberrant expression of CXCL1 is associated with the growth and progression of certain tumors. A naturally occurring processed form of CXCL1 has increased chemotactic activity. Alternate splicing results in coding and non-coding variants of this gene.

C-X-C motif chemokine ligand 8 (CXCL8) is a protein that is encoded in humans by the CXCL8 gene. CXCL8 is a member of the CXC chemokine family and is a major mediator of the inflammatory response. The encoded protein is secreted primarily by neutrophils, where it serves as a chemotactic factor by guiding the neutrophils to the site of infection. This chemokine is also a potent angiogenic factor. The CXCL8 gene is believed to play a role in the pathogenesis of bronchiolitis, a common respiratory tract disease caused by viral infection.

The small proline rich protein 2C (SPRR2C) is a pseudogene.

The small proline rich protein 2D (SPRR2D) is a protein that is encoded in humans by the SPRR2D gene. SPRR2D is a cross-linked envelope protein of keratinocytes. It is a keratinocyte protein that first appears in the cell cytosol, but ultimately becomes cross-linked to membrane proteins by transglutaminase resulting in the formation of an insoluble envelope beneath the plasma membrane.

The serpin family B member 3 (SERPINB3) is a protein that is encoded in humans by the SERPINB3 gene. SERPINB3 is believed to act as a papain-like cysteine protease inhibitor to modulate the host immune response against tumor cells. It also functions as an inhibitor of UV-induced apoptosis via suppression of the activity of c-Jun NH(2)-terminal kinase (JNK1). Diseases associated with SERPINB3 include squamous cell carcinoma and anus cancer. An important paralog of this gene is SERPINB4.

The serpin family B member 4 (SERPINB4) is a protein that is encoded in humans by the SERPINB4 gene. SERPINB4 is a member of the serpin family of serine protease inhibitors and is highly expressed in many tumor cells. SERPINB4, along with SERPINB3, can be processed into smaller fragments that aggregate to form an autoantigen in psoriasis, probably by causing chronic inflammation.

Peptidase inhibitor 3 (PI3) is a protein that is encoded in humans by the PI3 gene. PI3 is an elastase-specific inhibitor that functions as an antimicrobial peptide against Gram-positive and Gram-negative bacteria, and fungal pathogens. The protein contains a WAP-type four-disulfide core (WFDC) domain, and is thus a member of the WFDC domain family. Most WFDC gene members are localized in two chromosomal clusters: centromeric and telomeric. The PI3 gene belongs to the centromeric cluster. Expression of PI3 is upregulated by bacterial lipopolysaccharides and cytokines.

Lipocalin 2 (LCN2) is a protein that is encoded in humans by the LCN2 gene. LCN2 is a protein that belongs to the lipocalin family. Members of this family transport small hydrophobic molecules such as lipids, steroid hormones and retinoids. LCN2 is a neutrophil gelatinase-associated lipocalin and plays a role in innate immunity by limiting bacterial growth as a result of sequestering iron-containing siderophores. The presence of LCN2 in blood and urine is an early biomarker of acute kidney injury. LCN2 is thought to be involved in multiple cellular processes, including maintenance of skin homeostasis, and suppression of invasiveness and metastasis. Mice lacking the LCN2 gene are more susceptible to bacterial infection than wild type mice.

“Transglutamase 1 (TGM1) is a protein that is encoded in humans by the TGM1 gene. TGM1 is an enzyme that catalyzes the addition of an alkyl group from an alkylamine to a glutamine residue of a protein, forming an alkylglutamine in the protein. This protein alkylation leads to crosslinking of proteins and catenation of polyamines to proteins. Mutations in the TGM1 gene have been associated with autosomal recessive lamellar ichthyosis (LI) and nonbullous congenital ichthyosiform erythroderma.

Defensin beta 1 (DEFB1) is a protein that is encoded in humans by the DEFB1 gene. Defensins form a family of microbicidal and cytotoxic peptides made by neutrophils. Members of the defensin family are highly similar in protein sequence. DEFB1 is an antimicrobial peptide implicated in the resistance of epithelial surfaces to microbial colonization. The DEFB1 gene maps in close proximity to defensin family member defensin alpha 1 and has been implicated in the pathogenesis of cystic fibrosis.

The marker of proliferation Ki-67 (MKI67) is a protein that is encoded in humans by the MKI67 gene. MKI67 is a nuclear protein that is associated with and may be necessary for cellular proliferation.

Additional information such as database accession numbers, relevant publications, primer and/or antibody information are summarised in Tables 1 and 2 provided below.

TABLE 1
List of marker genes. Shown are the NCBI reference ID of the corresponding mRNA,
doi numbers of reference publications and commercially available antibodies.

| | NCBI | | |
|---|---|---|---|
| Gene name | RefSeq | ref DOI | Antibody |
| keratin 1 (KRT1) | NM_006121 | 10.1371/journal.pone.0180853 | HPA017917 Atlas Antibodies |
| keratin 10 (KRT10) | NM_000421.3 | 10.1111/j.1365-2133.2006.07403.x | ab76318 Abcam |
| filaggrin (FLG) | NM_002016.1 | 10.1371/journal.pone.0180853 | PRB-417P-100 Convance |
| loricrin (LOR) | NM_000427 | 10.1371/journal.pone.0180853 | HPA076123 Atlas Antibodies |
| keratin 5 (KRT5) | NM_000424.3 | 10.1038/sj.emboj.7600183 | ab52635 Abcam |
| keratin 14 (KRT14) | NM_000526.4 | 10.1091/mbc.E10-08-0703 | ab7800 Abcam |
| S100 calcium binding | NM_001319196.1 | 10.1016/j.febslet.2010.12.037 | HPA024372 Atlas Antibodies |
| protein A8 (S100A8) | | | |
| S100 calcium binding | NM_002965.3 | 10.1016/j.febslet.2010.12.037 | HPA004193 Atlas Antibodies |
| protein A9 (S100A9) | | | |
| C-X-C motif chemokine | NM_001511.3 | 10.1186/1471-2164-14-527 | NBP2-16748 Novus Biologicals |
| ligand 1 (CXCL1) | | | |
| C-X-C motif chemokine | NM_000584.3 | 10.1038/jid.2010.340 | HPA057179 Atlas Antibodies |
| ligand 8 (CXCL8) | | | |
| small proline rich | NR_003062.1 | 10.1186/1471-2164-14-527 | Not found |
| protein 2C (SPRR2C) | | | |
| small proline rich | NM_006945.4 | 10.1038/jid.2010.340 | 23046-1-AP Proteintech |
| protein 2D (SPRR2D) | | | |
| serpin family B | NM_006919.2 | 10.1186/1471-2164-14-527 | Not found |
| member 3 (SERPINB3) | | | |
| serpin family B | NM_002974.3 | 10.1186/1471-2164-14-527 | Thermo Scientific PA5-62118 |
| member 4 (SERPINB4) | | | |
| peptidase inhibitor 3 (PI3) | NM_002638.3 | 10.1186/1471-2164-14-527 | HPA017737 Atlas Antibodies |
| lipocalin 2 (LCN2) | NM_005564.4 | 181/10/7420 [pii] | HPA002695 Atlas Antibodies |
| transglutaminase 1 (TGM1) | NM_000359 | 10.1371/journal.pone.0180853 | HPA040171 Atlas Antibodies |
| keratin 6A (KRT6A) | NM_005554.3 | 10.1111/j.1365-2133.2006.07403.x | ThermoScientific MAI-35561 |
| | | 10.1111/exd.13023 | |
| keratin 16 (KRT16) | NM_005557.3 | 10.1111/exd.13023 | HPA000539 Atlas Antibodies |
| defensin Beta 1 (DEFB1) | NM_080389.2 | 10.1016/j.jaci.2016.06.038 | Thermo Scientific PA5-51286 |
| involucrin (IVL) | NM_005547.3 | 10.1007/s12013-012-9499-y. | ab20202 Abcam |
| S100 calcium binding | NM_002963.3 | 10.1016/j.jdermsci.2015.05.007 | HPA006997 Atlas Antibodies |
| protein A7 (S100A7) | | | |
| marker of proliferation | NM_002417.4 | 10.1007/s00403-010-1046-3 | ab15580 Abcam |
| Ki-67 (MKI67) | | | |

TABLE 2
List of marker genes. Shown are commercially available TaqMan probes (Thermo
Fisher Scientific) for real-time qRT-PCR analysis and oligonucleotide primer pairs (SEQ ID
NOs: 8 to 41) for RT-PCR analysis derived from the Harvard primer data bank (see the
World Wide Web at pga.mgh.harvard.edu/primerbank/).

| | TaqMan Gene | Primer sequences |
|---|---|---|
| | Expression Array | Forward and reverse 5′ to 3′ |
| Gene name | (Thermo Fisher Scientific) | (Harvard primer data bank) |
| keratin 1 (KRT1) | Hs01549614_g1 | fw 5′ TGAGCCGCATTCTGAACGAG |
| | | rv 5′ GATGACTGCGATCCAGAGGA |
| keratin 10 (KRT10) | Hs00166289_m1 | fw 5′ GGTGGGAGTTATGGAGGCAG |
| | | rv 5′ CGAACTTTGTCCAAGTAGGAAGC |
| filaggrin (FLG) | Hs00856927_g1 | fw 5′ GCACTCGTCATGCAGAGACTT |
| | | rv 5′ GACCCTCGGTTTCCACTGT |
| loricrin (LOR) | Hs01894962_s1 | fw 5′ CTCCTGTGGGTTGTGGAAAGA |
| | | rv 5′ TGGAACCACCTCCATAGGAAC |
| keratin 5 (KRT5) | Hs00361185_m1 | fw 5′ AGGAGTTGGACCAGTCAACAT |
| | | rv 5′ TGGAGTAGTAGCTTCCACTGC |
| keratin 14 (KRT14) | Hs00265033_m1 | fw 5′ TGAGCCGCATTCTGAACGAG |
| | | rv 5′ GATGACTGCGATCCAGAGGA |
| S100 calcium binding protein A8 | Hs00374264_g1 | fw 5′ ATGCCGTCTACAGGGATGAC |
| (S100A8) | | rv 5′ ACTGAGGACACTCGGTCTCTA |
| S100 calcium binding protein A9 | Hs00610058_m1 | fw 5′ GGTCATAGAACACATCATGGAGG |
| (S100A9) | | rv 5′ GGCCTGGCTTATGGTGGTG |
| C-X-C motif chennokine ligand 1 | Hs00236937_m1 | not available |
| (CXCL1) | | |
| C-X-C motif chennokine ligand 8 | Hs00174103_m1 | not available |
| (CXCL8) | | |
| small proline rich protein 2C | Hs00272438_m1 | not available |
| (SPRR2C) | | |
| small proline rich protein 2D | Hs03056964_s1 | not available |
| (SPRR2D) | | |
| serpin family B member 3 | Not available | fw 5′ CGCGGTCTCGTGCTATCTG |
| (SERPINB3) | | rv 5′ ATCCGAATCCTACTACAGCGG |
| serpin family B member 4 | Hs01691258_g1 | fw 5′ CTGGGTGGAAAGTCAAACGAA |
| (SERPINB4) | | rv 5′ TGTCGTATCATTGCCAATAGTCC |
| peptidase inhibitor 3 (PI3) | Hs00160066_m1 | fw 5′ CACGGGAGTTCCTGTTAAAGG |
| | | rv 5′ TCTTTCAAGCAGCGGTTAGGG |
| lipocalin 2 (LCN2) | Hs01008571_m1 | fw 5′ GAAGTGTGACTACTGGATCAGGA |
| | | rv 5′ ACCACTCGGACGAGGTAACT |
| transglutaminase 1 (TGM1) | Hs01070310_m1 | fw 5′ ATCATCGGCAAGTTTCAGTTCA |
| | | rv 5′ TCCCGTAGTAAATTCTCCCAGAC |
| keratin 6A (KRT6A) | Hs04194231_s1 | not available |
| keratin 16 (KRT16) | Hs00373910_g1 | fw 5′ GACCGGCGGAGATGTGAAC |
| | | rv 5′ CTGCTCGTACTGGTCACGC |
| defensin Beta 1 (DEFB1) | Hs00414476_m1 | fw 5′ AGACTTGTGCTGCTATTAGCCG |
| | | rv 5′ GGGCAGTCCCATAACCACATA |
| involucrin (IVL) | Hs00846307_s1 | fw 5′ GACTGCTGTAAAGGGACTGCC |
| | | rv 5′ CATTCCCAGTTGCTCATCTCTC |
| S100 calcium binding protein A7 | Hs01923188_u1 | fw 5′ ACGTGATGACAAGATTGACAAGC |
| (S100A7) | | rv 5′ GCGAGGTAATTTGTGCCCTTT |
| marker of proliferation Ki-67 | Hs04260396_g1 | not found |
| (MKI67) | | |

The term “at least one”, as used herein, encompasses also at least two, at least three, at least four, at least five, at least six, at least seven, at least eight, at least nine, at least ten different amino acids or more, such as at least eleven, at least 12, at least 13, at least 14, at least 15, at least 16, at least 17, at least 18 or all 19 of the recited markers. It will be appreciated by the skilled person that this term further encompasses exactly one, exactly two, exactly three, exactly four, exactly five, exactly six, exactly seven, exactly eight, exactly nine, exactly ten, exactly eleven, exactly 12, exactly 13, exactly 14, exactly 15, exactly 16, exactly 17, exactly 18 or exactly all 19 markers from the recited list of markers. Particularly preferred markers are selected from KRT1, KRT10, IVL, FLG, LOR, and TGM1 as markers for differentiation of keratinocytes and/or from KRT6, KRT16 and MK167 as markers of proliferating keratinocytes.

The skilled person is aware of suitable methods of determining whether at least one of the above recited markers are expressed by the cells. Such methods include, without being limiting, determining the expression of a marker on the amino acid level as well as on the nucleic acid level, as defined herein above. For example, such methods include Western Blot analysis, qRT-PCR analysis, immunohisto- or cytochemistry analysis, as well as microarray or RNA sequencing analysis. It is particularly preferred that the expression of said at least one marker is determined by Western Blot analysis or qRT-PCR analysis.

Some of the above recited genes are known as differentiation markers of keratinocytes. Generally, the expression of KRT1, KRT10, FLG and LOR is known to increase upon keratinocyte differentiation, while KRT4 and KRT15 are typically down-regulated upon differentiation. In accordance with the present invention, the following expression profiles are of particular relevance:
(i) Analysis of the expression of at least one marker selected from KRT1, KRT10, FLG, LOR, IVL, TGM1 and/or at least one marker selected from KRT4 and KRT15 in normal, healthy keratinocytes cultured in vitro in a 2D cell culture. An increase in the expression of KRT1, KRT10, FLG, LOR, IVL and/or TGM1 and/or a decrease in the expression of KRT4 and/or KRT15 after stimulation with a test compound as compared to untreated normal, healthy keratinocytes indicates an increased rate of differentiation. This model can, for example, serve to identify test compounds suitable as lead compounds for the treatment of skin conditions associated with an altered differentiation of keratinocytes, such as psoriasis or dermatitis, preferably atopic dermatitis.
(ii) Analysis of the expression of at least one marker selected from KRT1, KRT10, FLG and LOR and/or at least one marker selected from IVL, S100A8, DEFB1, KRT6, KRT16 and MK167 in a psoriatic keratinocyte model (for example a model generated by stimulation with the cytokines TNF-α and IL-17) cultured in vitro in a 2D cell culture. An increase in the expression of KRT1, KRT10, FLG and/or LOR and/or a decrease in the expression of IVL, S100A8, DEFB1, KRT6, KRT16 and/or MK167 after stimulation with a test compound as compared to untreated psoriatic keratinocytes of the same model indicates an increased rate of differentiation, and, hence, an improvement in differentiation accompanied by a reduction of proliferation, as well as an improvement in disease conditions. This model can serve in particular to identify test compounds suitable as lead compounds for the treatment of psoriasis.
(iii) Analysis of the expression of at least one marker selected from KRT1, KRT10, FLG and TGM1 and/or at least one marker selected from S100A8, S100A9, CXCL1, CXCL8/IL-8, SPRR2C, SPRR2D, SERPINB3, SERPINB4, PI3, LCN2, IVL, KRT6, KRT16 and MK167 in the skin of psoriasis patients (e.g. skin biopsies). An increase in the expression of KRT1, KRT10, FLG and/or TGM1, and/or a decrease in the expression of S100A8, S100A9, CXCL1, CXCL8/IL-8, SPRR2C, SPRR2D, SERPINB3, SERPINB4, PI3, LCN2, IVL, KRT6, KRT16 and/or MK167 after stimulation with a test compound as compared to untreated skin of psoriasis patients indicates an increased rate of differentiation and, hence, an improvement in differentiation accompanied by a reduction of proliferation, as well as an improvement in the psoriasis phenotype. This model can serve in particular to identify test compounds suitable as lead compounds for the treatment of psoriasis.
(iv) Analysis of the expression of at least one marker selected from KRT1, KRT10, FLG and LOR and/or at least one marker selected from KRT4, KRT15, KRT6 and KRT16 in normal, healthy keratinocytes cultured in vitro in a 2D cell culture. A decrease in the expression of KRT1, KRT10, FLG and/or LOR and/or an increase in the expression of KRT4, KRT15, KRT6 and/or KRT16 after stimulation with a test compound as compared to untreated normal, healthy keratinocytes indicates a decreased rate of differentiation accompanied by an enhanced proliferation. This model can, for example, serve to identify test compounds suitable as lead compounds for promoting wound healing, for example after skin injury or in cases of impaired would healing.
(v) Analysis of the expression of at least one marker selected from KRT1, KRT10, FLG and LOR and/or at least one marker selected from KRT4, KRT15, KRT6 and KRT16 in keratinocytes obtained from wounds of patients. A decrease in the expression of KRT1, KRT10, FLG and/or LOR and/or an increase in the expression of KRT4, KRT15, KRT6 and/or KRT16 after stimulation with a test compound as compared to untreated keratinocytes obtained from wounds of patients indicates a decreased rate of differentiation, accompanied by an enhanced proliferation. This model can also serve to identify test compounds suitable as lead compounds for promoting wound healing, for example after skin injury or in cases of impaired would healing.

Preferably, the expression level of all of the markers of the marker combinations cited in (i) to (v) above are determined. It will be appreciated that the expression level in the untreated controls can either be determined in parallel, subsequently or prior to the determination of the expression level in the sample stimulation with the test compound. In addition, said determination in untreated controls can be carried out once to provide a reference value for future use, or may be carried out each time the method is carried out. It is also envisaged that previously published data are relied on as reference value for the control.

In a further preferred embodiment of the method of the present invention, the method further comprises optimising the pharmacological properties of the compound identified. Accordingly, the compound identified by the present methods may be regarded as “lead compound”, from which further modified and potentially improved compounds can be developed.

The identified lead compound may for example be optimized by modifying the compound to achieve: (i) modified spectrum of activity, (ii) improved potency, (iii) decreased toxicity (improved therapeutic index), (iv) decreased side effects, (v) modified onset of action, duration of effect, (vi) modified physico-chemical parameters (solubility, hygroscopicity, color, taste, odor, stability, state), and/or (vii) optimised application form and route. Means and methods to achieved such modification include, without being limiting (a) esterification of carboxyl groups, (b) esterification of hydroxyl groups with carboxylic acids, (c) esterification of hydroxyl groups to, e.g. phosphates, pyrophosphates or sulfates or hemi-succinates, (d) formation of pharmaceutically acceptable salts, (e) formation of pharmaceutically acceptable complexes, (f) synthesis of pharmacologically active polymers, (g) introduction of hydrophilic moieties, (h) introduction/exchange of substituents on aromates or side chains, change of substituent pattern, (i) modification by introduction of isosteric or bioisosteric moieties, (j) synthesis of homologous compounds, (k) introduction of branched side chains, (l) conversion of alkyl substituents to cyclic analogues, (m) derivatisation of hydroxyl groups to ketales, acetales, (n) N-acetylation to amides, phenylcarbamates, (o) synthesis of Mannich bases, imines, and/or (p) transformation of ketones or aldehydes to Schiff's bases, oximes, acetales, ketales, enolesters, oxazolidines, thiazolidines or combinations thereof.

The various steps recited above are generally known in the art.

Both methods of the invention (herein also referred to as the “screening methods of the invention”) are suitable to identify compounds that are capable of altering the differentiation of keratinocytes.

In a preferred embodiment of both methods, an increase in the amount of LRRC8A protein or LRRC8A transcript after contacting the keratinocytes with the test compound and/or an increase in the activity of (a) VRAC(s) comprising LRRC8A after contacting the keratinocytes with the test compound indicates that the test compound is a compound suitable for use in the treatment and/or prevention of a skin condition associated with an altered differentiation of keratinocytes. In a particularly preferred embodiment of the methods of the invention, the skin condition associated with an altered differentiation of keratinocytes is psoriasis or dermatitis, preferably atopic dermatitis. Definitions and preferred embodiments of these conditions have been provided herein above.

In an alternative, preferred embodiment of both methods, a decrease in the amount of LRRC8A protein or LRRC8A transcript after contacting the keratinocytes with the test compound and/or a decrease in the activity of (a) VRAC(s) comprising LRRC8A after contacting the keratinocytes with the test compound indicates that the test compound is a compound suitable for use in the treatment and/or prevention of a skin condition selected from skin injury and impaired wound healing.

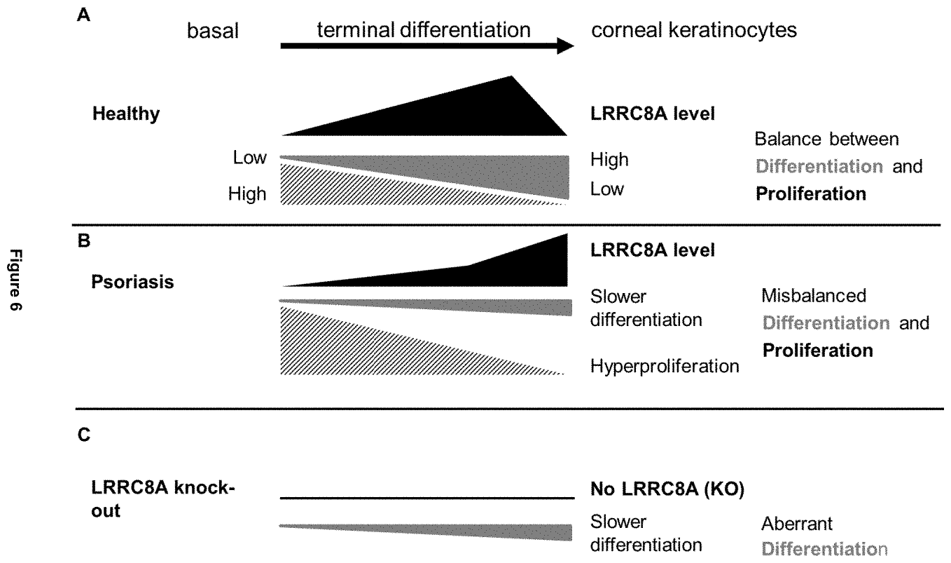

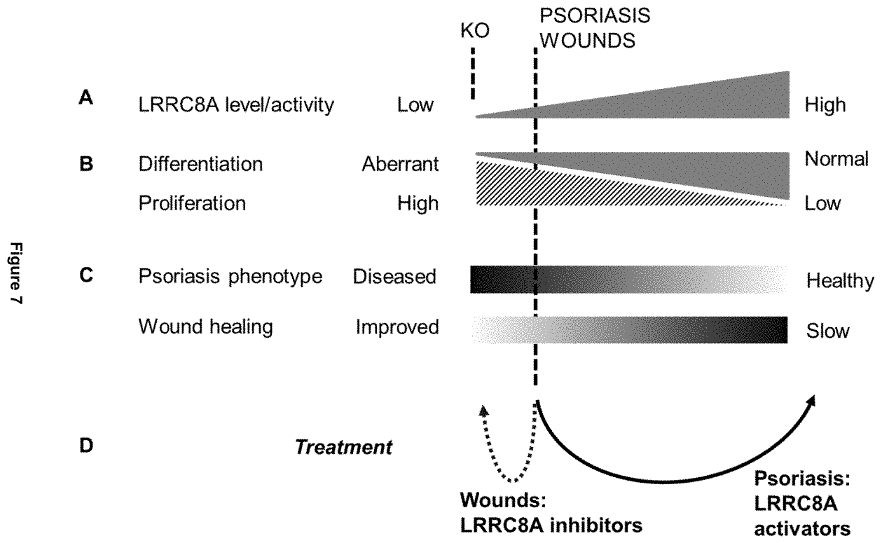

Wound healing is characterized by a decreased differentiation of keratinocytes, on the one hand, and by fast proliferation of keratinocytes, on the other hand. While in psoriasis and similar skin conditions an increase in differentiation and a decrease in proliferation is desirable, the opposite is desirable in promoting wound healing. Here, it is beneficial to further promote this type of epidermal change, such that differentiation is further slowed down, whereas proliferation is even more enhanced. As shown in the appended examples and depicted in FIGS. 6 and 7, the inhibition of LRRC8A was found to reduce differentiation of keratinocytes. Thus, it is beneficial to promote wound healing with LRRC8A inhibitors, which result in less differentiation and more proliferation of keratinocytes.

Accordingly, the present invention further relates to an inhibitor of the leucine-rich repeat-containing protein 8A (LRRC8A) for use in the treatment and/or prevention of a skin condition selected from skin injury and impaired wound healing.

The term “inhibitor”, as used in this embodiment, is defined as a compound suppressing or reducing the expression and/or activity of a target molecule, i.e. of LRRC8A. Preferably, the inhibitor mediates one or more of the following effects: (i) the expression, i.e. transcription and/or translation, of the gene encoding LRRC8A is suppressed or decreased, and (ii) LRRC8A performs its function, such as e.g. its biochemical and/or cellular function, with decreased efficiency in the presence of the activator.

Compounds falling in class (i) include compounds interacting with the transcriptional machinery and/or with the promoter of the LRRC8A gene and/or with expression control elements remote from the promoter such as enhancers. Also included are antisense constructs and constructs for performing RNA interference (e.g. siRNA, shRNA, miRNA) well known in the art (see, e.g. Zamore (2001) Nat. Struct. Biol. 8(9), 746; Tuschl (2001) Chembiochem. 2(4), 239), targeted to molecules that e.g. inhibit LRRC8A expression. Compounds of class (ii) decrease the function of the protein to be inhibited, in the present case of LRRC8A they decrease its VRAC activity as e.g. described in Example 5 below as well as its activity as a differentiation regulator of keratinocytes, as e.g. described in Example 6 below.

In accordance with the present invention, the term “inhibitor” encompasses both molecules that have a directly inhibiting effect on LRRC8A but also molecules that are indirectly inhibiting, e.g. by interacting for example with molecules that regulate LRRC8A expression or function. Accordingly, a molecule having a direct effect on LRRC8A necessarily will be an inhibitor, whereas a molecule having an indirect effect on LRRC8A can be a positive (i.e. activating) or negative (i.e. inhibiting) regulator, as long as the overall effect on LRRC8A is an inhibition. Preferably, the inhibitor acts directly on LRRC8A, more preferably it directly decreases the transcription and/or translation of LRRC8A.

The inhibitor, in accordance with the present invention, can be provided as any of the types of molecules defined herein above for the activator of LRRC8A, i.e. it can be provided as a small molecule, a proteinaceous compound or as a nucleic acid molecule. All of the general definitions and general embodiments provided herein above for these compounds, i.e. small molecules, proteinaceous compounds and nucleic acid molecules, apply mutatis mutandis, although it will be appreciated that the target of a compound that is to serve as an inhibitor of LRRC8A will have to be chosen differently from the target of a compound that serves as an activator of LRRC8A. Preferably, the inhibitor is an antibody that specifically recognises LRRC8A or a nucleic acid molecule that specifically removes or silences LRRC8A nucleic acid sequences, such as e.g. an siRNA, CRISPR-Cas9- and CRISPR-Cpf1-based constructs, as well as a meganuclease, zinc finger nuclease or transcription activator-like (TAL) effector (TALE) nuclease, or the inhibitor is a small molecule that specifically recognises LRRC8A. Also envisaged in accordance with the present invention are inhibitors, such as e.g. antibodies, interfering nucleic acid sequences or small molecule compounds that indirectly inhibit LRRC8A, e.g. by acting on upstream regulators of LRRC8A expression.

The term “inhibitor or LRRC8A”, as used herein, refers to a compound that reduces the biological function of LRRC8A to at least 50%, preferably to at least 75%, more preferred to at least 90% and even more preferred to at least 95% such as at least 98% or even at least 99%. Biological function denotes in particular any known biological function of LRRC8A or any combination thereof including functions elucidated in accordance with the present invention. Examples of said biological function are those described herein above with regard to the activator of LRRC8A. All these functions can be tested for by the skilled person either on the basis of common general knowledge or on the basis of the teachings of this specification, optionally in conjunction with the teachings of the documents cited therein.

The inhibitor in accordance with the present invention is for use in the treatment and/or prevention of a skin condition selected from skin injury and impaired wound healing. Skin injuries encompass wounds that go through the skin, such as cuts, lacerations, gashes and tears, as well as wounds that are on the surface of the skin, such as e.g. scrapes, abrasions, scratches and floor burns. The term “impaired wound healing”, as used herein, relates to a condition wherein wounds that are already present do not heal, leading to chronic wounds, or heal slowly.

All other definitions and preferred embodiments provided herein above with regard to the LRRC8A or the activator of LRRC8A but also the methods described above apply mutatis mutandis also to this inhibitor of LRRC8A.

In a preferred embodiment of the inhibitor for use according to the invention, the inhibitor is used in combination with one or more additional compounds selected from an inhibitor of LRRC8B, an inhibitor of LRRC8C, an inhibitor of LRRC8D, an inhibitor of LRRC8E, and an inhibitor of LRRC8F.

In a preferred embodiment of the inhibitor of the invention, (i) the inhibitor decreases the expression of LRRC8A; and/or (ii) the inhibitor decreases the activity of volume-regulated anion channels (VRACs) comprising LRRC8A.

Means and methods to determine whether the expression of LRRC8A and/or the activity of volume-regulated anion channels (VRACs) comprising LRRC8A have been decreased have been provided in detail herein above.

In an alternative embodiment, the present invention further relates to a method of treatment and/or prevention of a skin condition selected from skin injury and impaired wound healing, the method comprising administering an inhibitor of the leucine-rich repeat-containing protein 8A (LRRC8A) to a subject in need thereof. All definition and preferred embodiments provided herein with regard to the inhibitor of LRRC8A for use in accordance with the invention apply mutatis mutandis to this method of treatment.

In a further preferred that the LRRC8A and/or the activator for use according to the invention, or the inhibitor for use according to the invention, is comprised in a pharmaceutical composition.

In accordance with the present invention, the term “pharmaceutical composition” relates to a composition for administration to a patient, preferably a human patient. The pharmaceutical composition of the invention comprises the compound(s) recited above. The pharmaceutical composition of the present invention may, optionally and additionally, comprise a pharmaceutically acceptable carrier. By “pharmaceutically acceptable carrier” is meant a non-toxic solid, semisolid or liquid filler, diluent, encapsulating material or formulation auxiliary of any type. Examples of suitable pharmaceutically acceptable carriers are well known in the art and include sodium chloride solutions, such as phosphate-buffered sodium chloride solutions, water, emulsions, such as oil/water emulsions, various types of wetting agents, sterile solutions, organic solvents etc. Such pharmaceutically acceptable carriers often contain minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) peptides, e.g., polyarginine or tripeptides; proteins, such as serum albumin, gelatin, or further immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; sugar alcohols such as mannitol or sorbitol; counter-ions such as sodium; and/or non-ionic surfactants such as polysorbates, poloxamers, or PEG. Also chitosan may be comprised in the pharmaceutical composition, e.g. for use in delaying the release of the active ingredients upon administration.

The pharmaceutical composition may comprise further agents, or may be administered in conjunction (not necessarily at the same time) with further agents, depending on the intended use of the pharmaceutical composition, such as e.g. (a) the additional compounds described herein above targeting the remaining LRRC8 subtypes of the VRAC of interest; as well as (b) established therapeutics for the treatment of psoriasis, such as e.g. (i) topical agents such as corticosteroids, vitamin D analogues, topical retinoids and calcineurin inhibitors, (ii) systemic drugs such as methotrexate, ciclosporin, acitretin and, where allowed, fumaric acid esters, or (iii) biologics such as antibodies that target TNF-α, IL-17A or IL-12/IL-23; or (c) established therapeutics for the treatment of dermatitis, such as e.g. (i) topical agents such as hydrocortisone or calcineurin inhibitors, (ii) antibodies targeting interleukin signaling, or (iii) agents targeting the JAK-STAT pathway by using both orally and topically applied small-molecule JAK-1/2 inhibitors.

Administration of the pharmaceutical compositions of the invention may be effected by different ways, e.g. by topical (e.g. as creams, lotions, sprays, powders, ointments, drops or transdermal patch), intravenous, intraperitoneal, subcutaneous, intramuscular, intradermal, intranasal or intrabronchial administration, but also bucally or as an oral spray. Accordingly, it is preferred that the pharmaceutically acceptable carrier is a carrier suitable for these modes of administration.

Most preferably, the carrier is suitable for topical administration. In order to be suitable for topical administration, the carrier should be, or contain, a penetration enhancer. Penetration enhancers typically help to overcome the barrier properties of the skin or mucosal surfaces and facilitate the percutaneous absorption of an active agent added to the pharmaceutical formulations. Alternative names for penetration enhancers include e.g. permeation enhancers, sorption promoters and accelerants. Penetration enhancers act by interacting with molecules in the stratum corneum to modify its permeability to achieve delivery at therapeutically effective rates. Such permeation enhancers can be applied to the skin by pretreatment or concomitantly or jointly with the drug. Penetration enhancers as substances used in pharmaceutical preparation have to meet a set of qualitative criteria: they must not be toxic and should not irritate the skin, for example by inducing a sensitization or causing an allergic reaction; they should also be pharmacologically inert at the concentration required to exert adequate permeation action; their effect should be predictive and reversible and, preferably, immediate; and they should be readily incorporated into pharmaceutical preparations.

In general, penetration enhancers can be divided into several chemical classes: alcohols (e.g. ethanol, propylene glycol or lauryl alcohol), amides (e.g. I-dodecylazepan-2-one (Azone)), esters (e.g. isopropyl myristrate, dodecyl-2-dimethylaminopropanoate (DDAIP)), ether alcohols (e.g. Transcutol), fatty acids (e.g. linoleic acid, oleic acid), glycols (e.g. propylene glycol), pyrrolidones (e.g. N-methyl-2-pyrrolidone), sulphoxides (dimethyl sulphoxide), surfactants (e.g. sodium lauryl sulphate, alkyl trimethyl ammonium halides, tween 80, 2-nonyl-1,3-dioxolane (SEPA 009)), terpenes (e.g. eugenol, farnesol)⁴⁹.

Compositions comprising such carriers can be formulated by well known conventional methods. Generally, the formulations are prepared by contacting the components of the pharmaceutical composition uniformly and intimately with liquid carriers or finely divided solid carriers or both. Then, if necessary, the product is shaped into the desired formulation. Preferred modes of administration of the pharmaceutical compositions of the invention are by topical administration.

The pharmaceutical compositions can be administered to the subject at a suitable dose. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. The skilled person knows that the effective amount of a pharmaceutical composition administered to an individual will, inter alia, depend on the nature of the compound. For example, if said compound is a polypeptide or protein, the total pharmaceutically effective amount of pharmaceutical composition administered parenterally per dose will be in the range of about 1 μg protein/kg/day to 10 mg protein/kg/day of patient body weight, although, as noted above, this will be subject to therapeutic discretion. More preferably, this dose is at least 0.01 mg protein/kg/day, and most preferably for humans between about 0.01 and 1 mg protein/kg/day. If given continuously, the pharmaceutical composition is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. Furthermore, if for example said compound is an iRNA agent, such as an siRNA, the total pharmaceutically effective amount of pharmaceutical composition administered will typically be less than about 75 mg per kg of body weight, such as for example less than about 70, 60, 50, 40, 30, 20, 10, 5, 2, 1, 0.5, 0.1, 0.05, 0.01, 0.005, 0.001, or 0.0005 mg per kg of body weight. More preferably, the amount will be less than 2000 nmol of iRNA agent (e.g., about 4.4×1016 copies) per kg of body weight, such as for example less than 1500, 750, 300, 150, 75, 15, 7.5, 1.5, 0.75, 0.15, 0.075, 0.015, 0.0075, 0.0015, 0.00075 or 0.00015 nmol of iRNA agent per kg of body weight.

The length of treatment needed to observe changes and the interval following treatment for responses to occur appears to vary depending on the desired effect. The particular therapeutically effective amount for a given situation will readily be determined by routine experimentation and is within the skills and judgment of the ordinary clinician or physician.

The components of the pharmaceutical composition to be used for therapeutic administration must be sterile. Sterility is readily accomplished, for example, by filtration through sterile filtration membranes (e.g., 0.2 μm membranes).

The present invention further relates to a cosmetic method for treating the skin of an individual, the method comprising topically administering an effective amount of (i) leucine-rich repeat-containing protein 8A (LRRC8A); (ii) an activator of LRRC8A; (iii) LRRC8A and an activator of LRRC8A; or (iv) an inhibitor of LRRC8A.

The term “cosmetic method”, as used herein, relates to a method for improving the optical appearance and/or skin sensation of the skin of individuals affected by a skin condition. Said optical appearances include, without being limiting, scarring, e.g. as the result of wounds to the skin, redness, aging, dullness, blotchy skin, wrinkling, unevenness, shiny skin, oiliness and scaliness. Examples of skin sensations that can be treated include, without being limiting, stiff or tense skin, itching or burning sensations, as well as a feeling of soreness. To provide a non-limiting example, skin disorders such as psoriasis and dermatitis are often accompanied by a worsening of the appearance of the skin or nails of the affected individual and/or of the skin sensation experienced by the affected person, including without being limiting, redness, scaling, peeling, dryness, itching, burning, soreness, nail discoloration or nail crumbling. These cosmetic problems can be alleviated by the cosmetic method of the present invention comprising topically administering an effective amount of the compounds recited in (i), (ii) or (iii). On the other hand, skin injuries and wound healing are often accompanied by unwanted and unsightly changes of the skin as well as unwanted skin sensations including, without being limiting, scarring, stiffness, itching, dryness, maceration, swelling, tension, and pigmentation. Also these cosmetic problems can be alleviated by the cosmetic method of the present invention, wherein the method comprises topically administering an effective amount of the compound recited in (iv).

In accordance with this cosmetic method of the invention, the recited compounds are to be administered topically. Topical administration is well known in the art and relates to the application of a compound to body surfaces such as the skin or mucous membranes, preferably the skin. The compound can be provided in various forms for topical administration including, without being limiting, in form of creams, lotions, foams, gels, sprays, powders, ointments, drops, pastes, tinctures, and transdermal patches.

As discussed herein above and as summarized in FIGS. 6 and 7, it was surprisingly found that a knock-out of expression of the leucine-rich repeat-containing protein 8A (LRRC8A) resulted in altered differentiation of keratinocytes. Accordingly, compounds that influence the amount of LRRC8A expression and/or activity represent promising tools for improving the appearance of skin by steering keratinocyte differentiation in the desired direction: by enhancing differentiation, the negative effects of diseases such as psoriasis and dermatitis on the appearance of skin can be alleviated, while by reducing differentiation and promoting proliferation, the appearance of scarred skin can be improved.

Unless otherwise defined, all technical and scientific terms used herein have the same meaning as commonly understood by one of ordinary skill in the art to which this invention belongs. In case of conflict, the patent specification, including definitions, will prevail.

All the sequences accessible through the Database Accession Numbers cited herein are within the scope of the present invention and also include potential future updates in the database, in order to account for future corrections and modifications in the entries of the respective databases, which might occur due to the continuing progress of science.

All amino acid sequences provided herein are presented starting with the most N-terminal residue and ending with the most C-terminal residue (N→C), as customarily done in the art, and the one-letter or three-letter code abbreviations as used to identify amino acids throughout the present invention correspond to those commonly used for amino acids.

Regarding the embodiments characterised in this specification, in particular in the claims, it is intended that each embodiment mentioned in a dependent claim is combined with each embodiment of each claim (independent or dependent) said dependent claim depends from.

For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims. To give a non-limiting example, the combination of claims 10, 9 and 6 is clearly and unambiguously envisaged in view of the claim structure. The same applies for example to the combination of claims 10, 9 and 5, or the combination of claims 13, 12 and 11, etc.

The figures show:

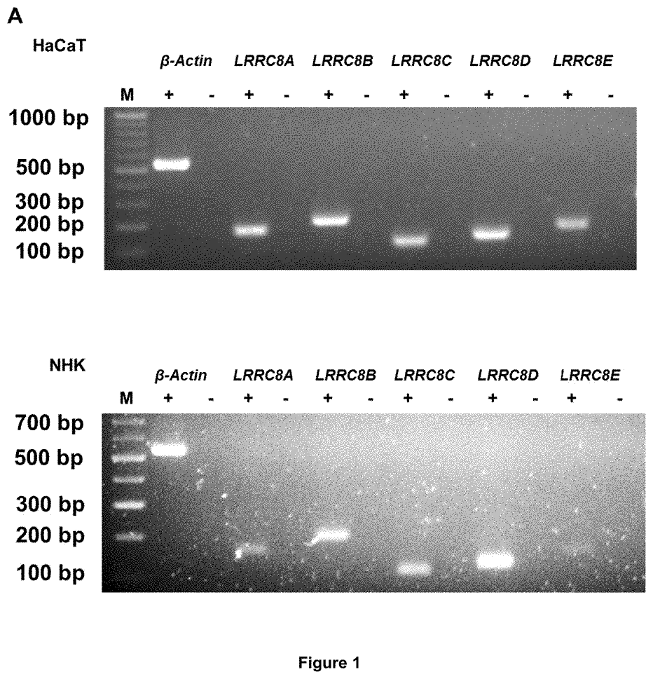

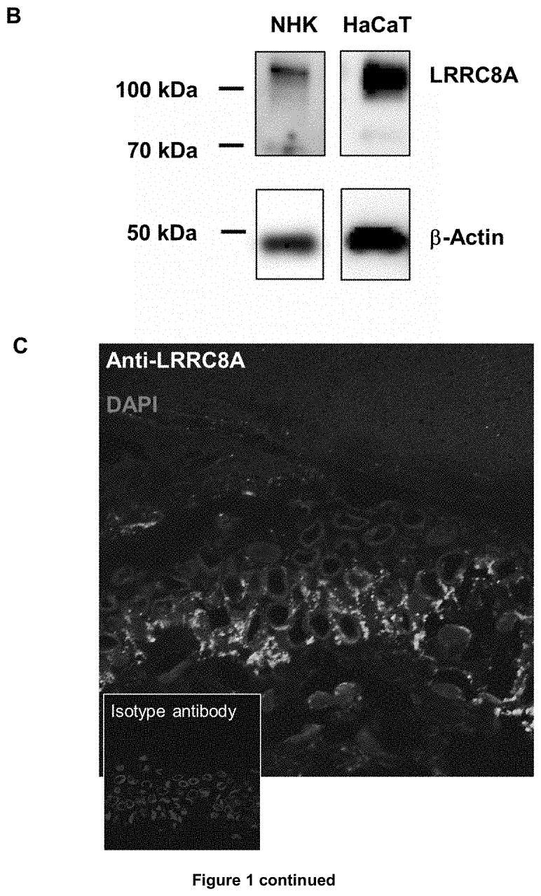

FIG. 1: LRRC8A expression in cultured keratinocytes and in human skin.

(A) RT-PCR analysis using total RNA isolated from HaCaT keratinocytes and normal human epidermal keratinocytes (NHK) revealed specific PCR products for all LRRC8 gene family members (LRRC8A-E). β-Actin-specific PCR product was obtained and served as loading control. No PCR product was obtained when reverse transcriptase (−RT) was omitted. M, Molecular weight standard. (B) Western blot analysis using whole cell extracts of normal human epidermal keratinocytes (NHK) and HaCaT keratinocytes showed a strong LRRC8A antibody signal at around 100 kDa, which is close to the calculated molecular mass of 94 kDa. β-Actin-specific antibody signal was obtained and served as loading control. M, Molecular weight standard. (C) Immunohistochemistry analysis using LRRC8A antibody revealed the existence of LRRC8A protein in human skin biopsies. Localization of LRRCA8 was visualized by using primary Anti-LRRC8A antibody and FITC-labeled secondary antibody. Green fluorescent FITC signal was preferentially detected in basal epidermal keratinocytes and declined towards the outer keratinocyte layers. Isotype antibody control showed no green fluorescent signal confirming specificity of the LRRC8A antibody. DNA was counterstained with DAPI to identify the cell nucleus. Note: The green fluorescent FITC signal is shown in white/light grey and DAPI is shown in dark grey in this picture.

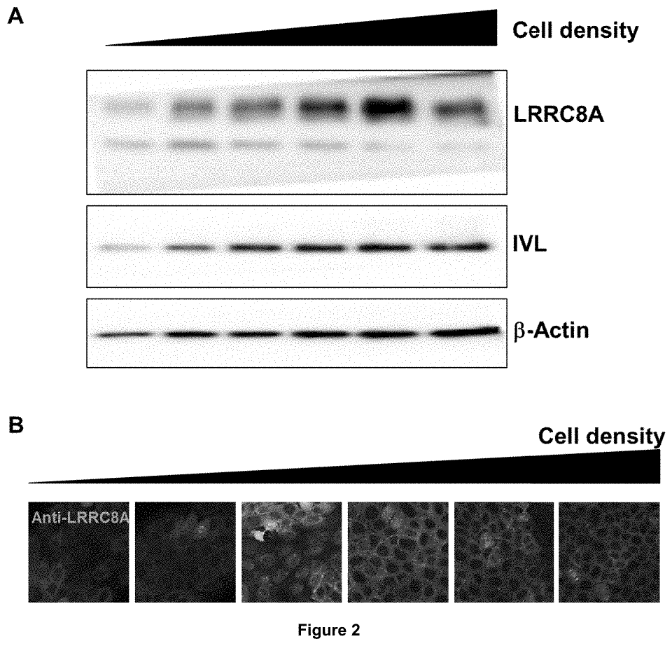

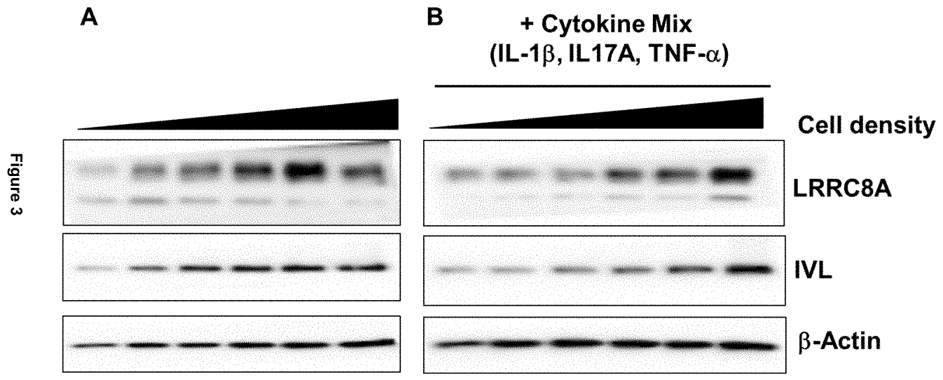

FIG. 2: Dynamic regulation of LRRC8A during keratinocyte differentiation.

Western blot (A) and immunofluorescence analysis (B) of HaCaT cells at different stages of differentiation showed that LRRC8A protein level (A) as well as membrane localization of LRRC8A (B) is first increased before it reached its maximum and then declined again when terminal differentiation is achieved. HaCaT cells were seeded at different cell densities (post-confluent growth) to induce differentiation. (A) Progressing differentiation was monitored by using an antibody against the differentiation marker involucrin (IVL) in whole cell extracts. 13-Actin served as loading control. (B) Membrane localization of LRRCA8 was visualized by using primary Anti-LRRC8A antibody and FITC-labeled secondary antibody. DNA was counterstained with DAPI to identify the cell nucleus. Note: The green fluorescent FITC signal is shown in white/light grey in this picture.

FIG. 3: LRRC8A expression in psoriatic keratinocytes and in psoriasis skin lesions.

Western Blot analysis of normally and abnormally differentiating HaCaT cells. HaCaT cells were seeded at different cell densities and cultivated in the presence (B) or absence (A) of pro-inflammatory cytokines (IL-1β, IL-17A, TNF-α) to mimic psoriatic conditions. Progressing normal (A) and abnormal (B) differentiation was monitored by using antibody against the differentiation marker involucrin (IVL) in whole cell extracts of HaCaT cells. By using Anti-LRRC8A antibody, a bell-shaped expression pattern of LRRC8A was observed during normal differentiation (A), whereas in psoriasis-like HaCaT cells, LRRC8A protein was detected much later and did not decrease at later stages of abnormal differentiation (B). (C) Punch biopsy (6 mm) from diseased, lesional skin (a, d) and non-lesional skin (d, e) of two different psoriasis vulgaris patients (patient 1: a, b; patient 2: d, e) or from healthy donors (c, f) were fixed in 4% PFA and paraffin embedded. 4 μm sections were processed routinely. For immunohistochemistry, primary anti-LRRC8A antibody was applied overnight after antigen retrieval with EDTA solution. Histofine Simple Stain AP Multi (Medac Diagnostika, Wedel, Germany) was used for detection, according to the manufacturer's instructions. Nuclei were stained with hematoxylin. Images were acquired by using a Nikon Eclipse slide scanning microscope. Black color indicates antibody binding. Bars represent 100 μm. Healthy human skin shows strong LRRC8A staining in the epidermis and especially in the basal layer (c, f). In contrast non-lesional skin of psoriatic patients shows reduced staining for LRRC8A (b, e) while hardly any (d) or very weak (a) staining can be detected in lesional psoriatic skin. Thus, LRRC8A expressions seems to be downregulated during the psoriatic inflammation.

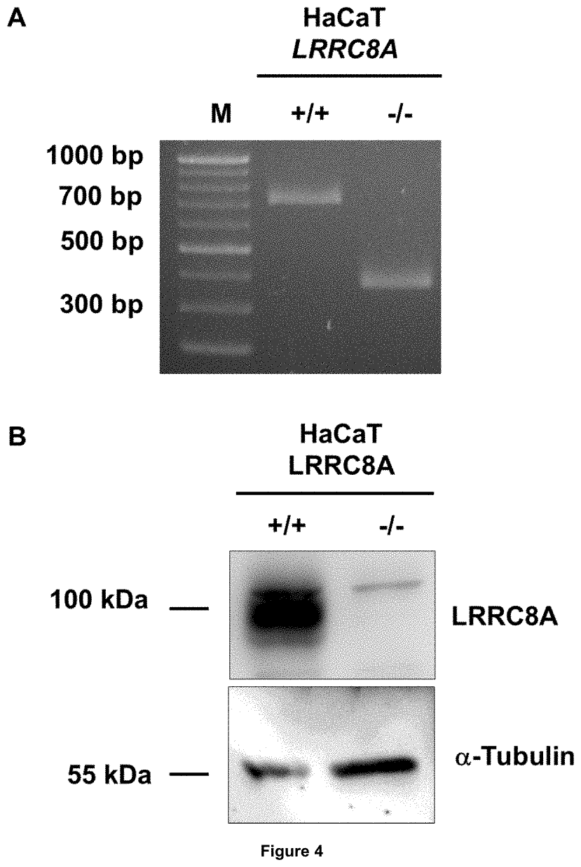

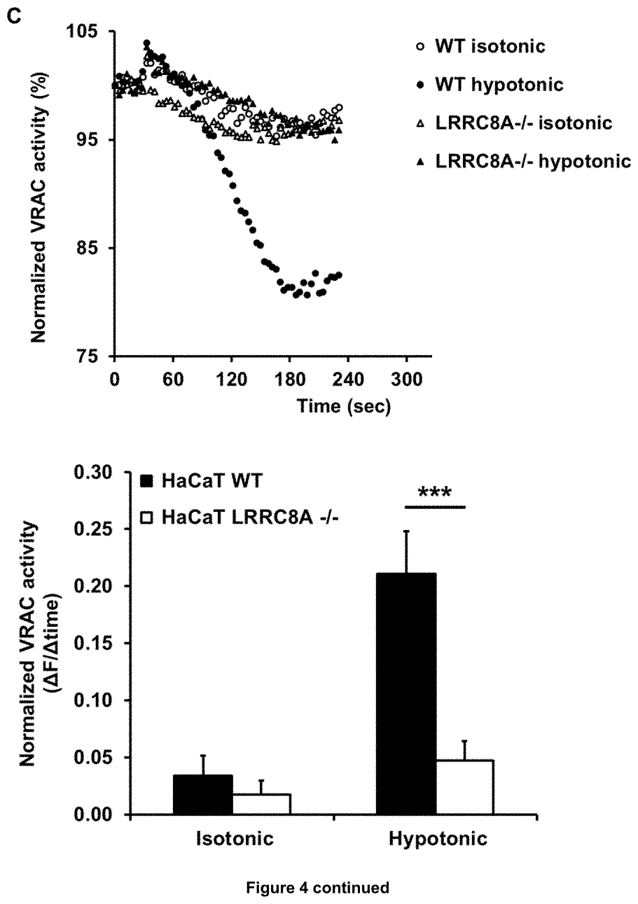

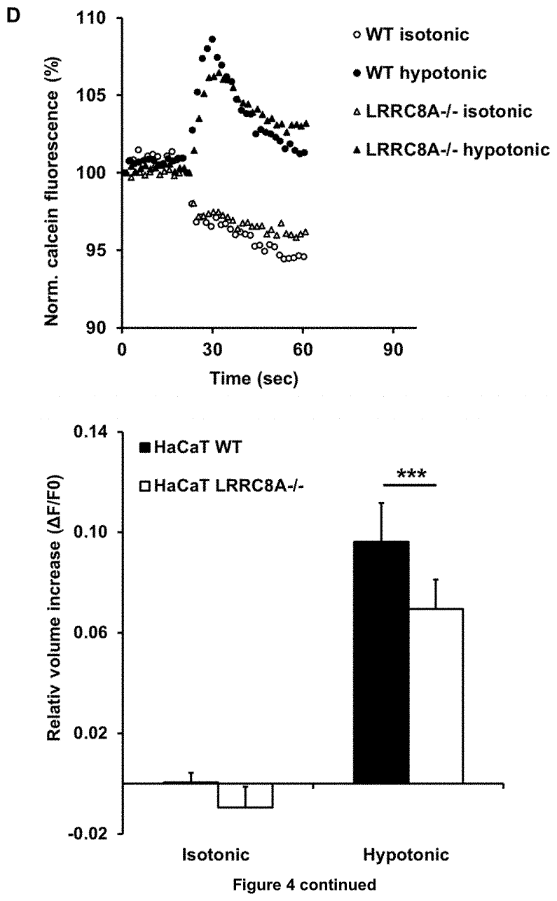

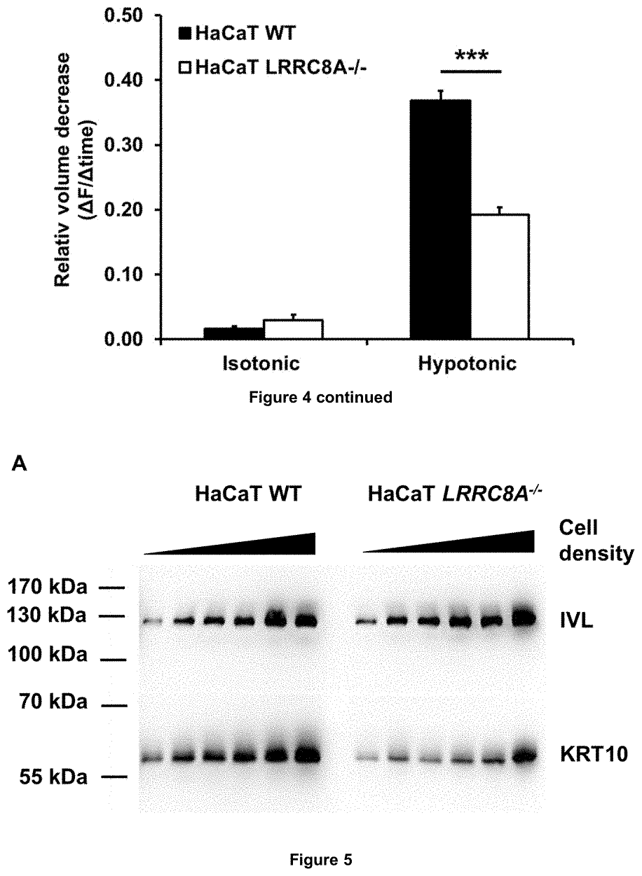

FIG. 4: Modified VRAC activity and RVD in HaCaT-LRRC8A^{-/-} cells generated by CRISPR-Cas9.

(A) PCR using genomic DNA as template and LRRC8A specific primer pairs results in a 700 bp PCR product in HaCaT wildtype cells (HaCaT-LRRC8A^{+/+}) and in a 400 bp PCR product in the HaCaT-LRRC8A^{-/-} cell clone, confirming the CRISPR-Cas9-induced 300 bp gene deletion of LRRC8A. M, Molecular weight standard. (B) Western blot analysis using whole cell extracts and LRRC8A antibody showed a strong LRRC8A antibody signal at around 100 kDa in HaCaT wildtype cells (HaCaT-LRRC8A^{+/+}), which is lacking in HaCaT-LRRC8A^{-/-} cells, confirming the absence of detectable LRRC8A protein in HaCaT-LRRC8A^{-/-} cells. Note the unspecific antibody signal at above 100 kDa, which is present both in wildtype and in knock out cells. β-Actin-specific antibody signal was obtained and served as loading control. M, Molecular weight standard. (C) Measuring VRAC activity using hsYFP in HaCaT cells. HaCaT wildtype (WT) and HaCaT LRRC8A^{-/-} cells were transduced with adenovirus containing hsYFP gene expression cassette, pre-incubated with isotonic buffer and then treated with iodide-containing isotonic or hypotonic buffer. hsYFP fluorescence was measured and plotted over time. Addition of isotonic buffer did not result in considerable quenching of hsYFP fluorescence whereas hypotonic iodide-containing buffer led to a fast I⁻ influx and strong I⁻-dependent hsYFP quenching. The speed of I⁻ influx (ΔF/Δtime) after hypotonic stimulation was quantified and used as a measure for VRAC activity. Exemplary raw data (left graph) and determined VRAC activity (depicted as mean value and standard deviation, bar diagram) are shown. Clearly, in HaCaT-LRRC8A^{-/-} cells VRAC activity after hypotonic stimulation was almost completely diminished. *** highly statistically significant, student's t-test, p<0.001. (D) Measuring cell volume changes using the volume-sensitive dye calcein in HaCaT cells. HaCaT wildtype (WT) and HaCaT LRRC8A^{-/-} cells were loaded with calcein, pre-incubated with isotonic buffer and stimulated with isotonic or hypotonic buffer. Calcein fluorescence was measured and plotted over time. Hypotonic stimulation of HaCaT cells led to a fast increase and subsequent decrease of calcein fluorescence, indicating cell swelling followed by compensatory RVD. The change of calcein fluorescence (ΔF=Fₘₐₓ−F₀) was normalized to baseline fluorescence (ΔF/F₀) and was used as a measure for relative volume increase (cell swelling). The speed of calcein fluorescence decrease (ΔF/Δtime) was used as a measure of relative volume decrease (RVD). Exemplary raw data (left graph) and calculated relative volume increase (top right) and decrease (bottom right) are shown as mean value and standard deviation. Cell swelling was only mildly affected whereas regulatory volume decrease (RVD) was drastically reduced in HaCaT-LRRC8A^{-/-} cells compared to HaCaT wildtype cells. *** highly statistically significant, student's t-est, p<0.001.

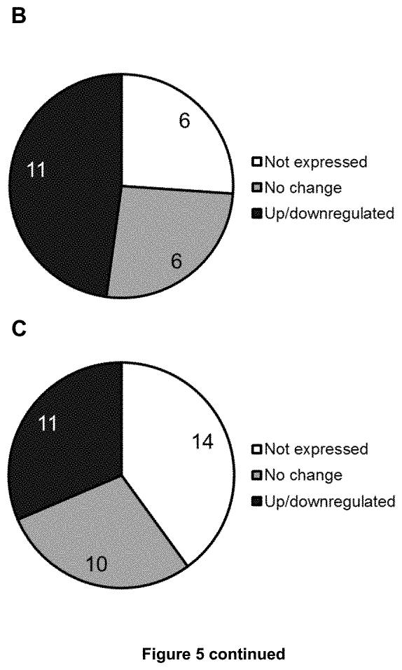

FIG. 5: LRRC8A-dependent changes of differentiation and gene expression in keratinocytes.

(A) Western Blot analysis of differentiating HaCaT wildtype cells (HaCaT WT) and HaCaT cells devoid of LRRC8A (HaCaT LRRC8A^{-/-}). HaCaT cells were seeded at different cell densities (post-confluent growth) to induce differentiation. Progression of differentiation was monitored by using antibodies against the differentiation markers involucrin (IVL) and keratin 10 (KRT10) in whole cell extracts. In cells lacking LRRC8A activity (HaCaT LRRC8A^{-/-}), IVL occurred in earlier stages of the differentiation process and KRT10 protein level did not continuously increase but stayed at a low level throughout the entire differentiation. (B, C) Pie charts to compare differentially regulated genes of the RNA sequencing data of HaCaT LRRC8A^{-/-} cells with published transcriptome studies that are related to psoriasis. Among the 23 genes, which were reported by Chiricozzi et al.⁵ to be differentially expressed in keratinocytes treated with IL-17, 6 genes were found to be not expressed at all in HaCaT cells, 6 genes not to be affected and almost 50% of the genes (i.e. 11 of the 23 genes, namely: IL1F9, PI3, LCN2, 1L8, S100A8, S100A9, SPRR2D, IL-1B, ALDH1A3, CXCL1, SAT1) to be either up- or downregulated in HaCaT-LRRC8A^{-/-} cells (B). Among the 35 genes, which were reported by Swindell et al.⁷ to be most strongly elevated in psoriasis lesions of patients, it was found by RNA sequencing that 14 genes were not expressed at all in HaCaT cells, 10 genes were not affected, whereas approx. 30% of the genes (i.e. 11 of the 35 genes, namely: TCN1, S100A7A, AKR1B10, PI3, S100A9, 1L36G, LCN2, OASL, IGFL1, KRT16, CXCL1) were also up- or downregulated in HaCaT-LRRC8A^{-/-} cells (C).

FIG. 6: Proposed model of dynamic LRRC8A expression during differentiation and proliferation of keratinocytes in healthy and diseased conditions.

(A) In healthy skin, keratinocytes develop gradually from proliferating basal cells into spinous, granular and corneal layers of the epidermis. This process is called terminal differentiation and requires a tight balance between proliferation and differentiation. At the beginning proliferation is high in basal cells, whereas differentiation is low (grey triangle). With proceeding terminal differentiation (black arrow), proliferation decreases (dashed triangle) whereas differentiation increases (grey triangle). Normal differentiation also requires dynamic change of LRRC8A expression. The level of LRRC8A (black triangle) first increases, then reaches its maximum and drops again at later stages of normal differentiation. Triangles indicate die increase/decrease of LRRC8A protein levels (black), differentiation (grey) and proliferation (dashed) during the process of terminal differentiation of keratinocytes (black arrow).

(B) In the skin disorder psoriasis, the balance between differentiation and proliferation is disturbed. Differentiation is slower (grey triangle), whereas proliferation is faster (hyperproliferation, dashed triangle). In this abnormal proliferation process, also LRRC8A expression is disturbed. LRRC8A levels (black triangle) are lower at early stages, increase much slower and do not decline again but instead stay higher in later stages compared to healthy skin.

(C) When LRRC8A is completely absent, e.g. in case of LRRC8A knock-out cells, or when LRRC8A levels are too low (black line) at early stages, e.g. in case of psoriasis, keratinocytes are not able to undergo their normal differentiation program (grey triangle). In summary, LRRC8A is required for normal epidermal differentiation. In contrast, in psoriasis LRRC8A levels are too low leading to abnormal differentiation. As a consequence, increasing LRRC8A expression or enhancing LRRC8A activity represents a novel approach for treatment of skin conditions that are related to disturbed differentiation, e.g. psoriasis.

FIG. 7: Use of LRRC8A activators or inhibitors for the treatment of psoriasis or wound healing, respectively.

The effect of different LRRC8A levels/ion channel activity (black triangle) on differentiation (grey triangle) and proliferation (dashed triangle) and its impact on psoriasis and wound healing are illustrated. Degree of change of psoriasis and wound healing is indicated by a black/grey color gradient, whereby grey indicates improvement and black indicates worsening.

(A) When LRRC8A activity/level is low (or even completely absent e.g. in case of knock-out cells), it causes aberrant differentiation and hyperproliferation (B), which are characteristics of both psoriasis and wound healing (C). To improve wound healing it is beneficial to further promote this type of epidermal change, meaning that proliferation should be even further increased and differentiation even more reduced (C+D). For treatment of psoriasis the opposite effect is beneficial, meaning that proliferation should be reduced and differentiation enhanced (C+D). This can be achieved by modulating LRRC8A activity: Inhibition of LRRC8A (E) will lead to less differentiation but more proliferation (B), thereby improving wound healing. In contrast, activation of LRRC8A (E) will lead to more differentiation and less proliferation (B), thereby improving psoriasis and similar disorders.

The following examples illustrate the invention:

### Example 1: Material and Methods

Cell Culture Conditions and Cell Cultivation

HaCaT cells were cultivated in Dulbecco's Modified Eagle Medium high glucose 4.5 g/L (PAA Laboratories) supplemented with 8% fetal calf serum (Biochrom) and 3.5 mM L-glutamine (PAA Laboratories) at 37° C. and 5% CO₂.

Normal human epidermal keratinocytes (NHK) are primary keratinocytes which were purchased from PromoCell or were isolated from human juvenile foreskin (according to 50). Cells were cultured in keratinocyte growth medium 2 (PromoCell) supplemented with keratinocyte growth medium 2 supplement Mix (PromoCell) containing 0.004 ml/ml bovine pituitary extract, 0.125 ng/ml epidermal growth factor, 5 μg/ml insulin, 0.33 μg/ml hydrocortisone, 0.39 μg/ml epinephrine, 10 μg/ml transferrin and 0.06 mM CaCl₂ up to a confluence of 80%.

Gene Expression Analysis Using RT-PCR

To determine whether and which LRRC8 family members (LRRC8A-E) are expressed in the keratinocyte cell line HaCaT and in normal human epidermal keratinocytes (NHK), RT-PCR was performed. For this purpose, total RNA from HaCaT and NHK cells was extracted by using NucleoSpin RNA II Kit (Macherey-Nagel). cDNA was synthesized by reverse transcription using 1 μg total RNA and Poly-dT primer (ProtoScript M-MuLV First Strand Synthesis Kit, New England Biolabs). PCR amplification of LRRC8A-E was performed using gene specific oligonucleotide primers and Phire Hot Start II PCR Master Mix (Thermo Scientific) including initial denaturation at 98° C. for 30 sec followed by 27 amplification cycles comprising denaturation at 98° C. for 5 sec, primer annealing at 55° C. for 5 sec, and elongation at 72° C. for 12 sec followed by a final elongation step at 72° C. for 1 min. PCR amplification products were separated on 2% agarose gels and visualized by Midori Green (Biozym) staining. The following sense and antisense oligonucleotides were used:

β-Actin fw
(SEQ ID NO: 42)
5′-GTGGGGCGCCCCAGGCACCA-3′;
β-Actin rv
(SEQ ID NO: 43)
5′-CTCCTTAATGTCACGCACGATTTC-3′;
LRRC8A fw
(SEQ ID NO: 44)
5′-CCTGCCTTGTAAGTGGGTCAC-3′;
LRRC8A rv
(SEQ ID NO: 45)
5′-CACAGCGTCCACGTAGTTGTA-3′;
LRRC8B fw
(SEQ ID NO: 46)
5′-CTGGCATAGAAAGCCCAACTT-3′;
LRRC8B rv
(SEQ ID NO: 47)
5′-CGATTTCAAGAGTGATGTGGGT-3′;
LRRC8C fw
(SEQ ID NO: 48)
5′-CTGGGGAAGTGTTTTGACTCTC-3′;
LRRC8C rv
(SEQ ID NO: 49)
5′-GGACCAGATTGGATGGTGTTG-3′;
LRRC8D fw
(SEQ ID NO: 50)
5′-GTGGTCTGTTTGCCAGTATTGC-3′;
LRRC8D rv
(SEQ ID NO: 51)
5′-CCCAAAGGAAATGTCGTTTGTTG-3′;
LRRC8E fw
(SEQ ID NO: 52)
5′-CAAGCAGTTCACGGAACAGC-3′;
LRRC8E rv
(SEQ ID NO: 53)
5′-GGGCCTCTGATAAGTTCTCCTG-3′.

Protein Detection Using Western Blot Analysis

Total protein was extracted by incubation of cells with RIPA lysis buffer (50 mM Tris pH 7.4, 150 mM NaCl, 1% Nonidet P-40, 0.5% Deoxycholate, 0.1% SDS, 1 mM EDTA) for 30 min at 4° C. Supernatant was centrifuged for 1 min and protein extracts were denatured by incubation with Laemmli sample buffer (0.5 M Tris pH 6.8, 4% SDS, 40% glycerol, 100 mM DTT, 0.08% bromophenol blue) for 10 min at 95° C. Protein concentration was determined by BCA assay (Thermo Fisher). 10-15 μg total protein was separated on 7.5% or 12.5% SDS-PAA gels and analysed by western blot using specific antibodies against LRRC8A (Novus NBP2-32158 1:500), Cytokeratin 10 (abcam ab76318, 1:10000), Involucrin (abcam ab20202, 1:10000), β-Actin (Sigma Aldrich A1978, 1:10000) and α-Tubulin (Sigma Aldrich T9026, 1:5000) and species specific secondary antibodies (a-mouse IgG, a-rabbit IgG, VWR, 1:5000). Protein was visualized by ECL reagent (Merck Chemicals) and sizes of proteins were estimated by comparison with protein marker IV (10-170 kDa) (PeqLab).

Immunohistology and Immunofluorescence Staining of Skin Specimen

Healthy individuals were recruited and gave written informed consent. The study was approved by the ethics committee of the Clinic of the Goethe-University (116/11); the Declaration of Helsinki protocols were followed. Punch biopsies (6 mm) were taken. For immunohistochemistry they were fixed in 4% PFA, paraffin embedded and cut into 4 μm sections. Paraffin sections were processed routinely⁵¹. Primary antibody (anti-Swell Novus 1:100; also available under the name “anti-LRRC8A NBP2-32158” from Novus Biologicals)) or rabbit isotype control antibody (#3900 Cell Signaling, 1:2,5) was applied overnight after antigen retrieval with citrate solution pH 6. Histofine Simple Stain AP Multi (Medac Diagnostika) was used for detection, according to the manufacturer's instructions. Images were acquired by using a Nikon Eclipse slide scanning microscope. For immunofluorescence staining biopsies were collected in TissueTek OCT (Sakura) and cut into 8 μm cryosections, fixed in methanol. Specimens were blocked with 5% goat serum/TBS-T and incubated overnight at 4° C. with primary anti-Swell1 Novus 1:100 or isotype antibody (#3900 Cell Signaling, 1:2,5). After washing with TBS, samples were incubated with AlexaFluor488 labeled secondary antibody (1:1000 LifeTechnologies) and nuclei were stained with DAPI. Confocal images were generated using a ZeissLSM510 microscope⁴.

Immunofluorescence Staining of Cells

HaCaT cells were seeded on glass slides, fixed in methanol and permeabilized with TBS-T. Specimens were blocked with 5% goat serum/TBS-T and incubated overnight at 4° C. with primary or isotype antibodies. After washing, samples were incubated with AlexaFluor488 labeled secondary antibody and nuclei were stained with DAPI. Confocal images were generated using a ZeissLSM510 microscope⁵².

Differentiation of Keratinocytes In Vitro

Differentiation of HaCaT cells was initiated by post-confluence growth. Therefore HaCaT cells were cultivated in increasing cell numbers (0.1 up to 1.0*10⁶ cells/6 well) for 48 h resulting in sub-confluent to post-confluent cell density as described in Buerger C. et al.⁴. Then, cells were harvested and used for RNA extraction, protein extraction or were fixed for immunofluorescence analysis. If indicated, cells were treated with a mix of inflammatory cytokines, consisting of IL-1b, IL-17A and TNF-α (from Peprotech; 20 ng/ml each) for the indicated period. Then, cells were harvested and used for RNA extraction, protein extraction.

Generation of LRRC8A Knock-Out Cell Lines by CRISPR/Cas9

To generate monoclonal HaCaT cells devoid of the LRRC8A gene, HaCaT cells were transduced with adenovirus Ad5-CMV-Cas9-wt-2A-OFP (produced by Sirion Biotech) delivering a gene expression cassette encoding for Cas9 nuclease and orange fluorescent protein under the control of the CMV promoter and with adenovirus Ad5-U6-sgRNA-G-SWELL1-U6-sgRNA-G-SWELL4 (produced by Sirion Biotech) delivering a gene expression cassette encoding two single guide RNAs targeting different positions of LRRC8A under the control of the U6 promoter. The sequence of used sgRNAs are as followed:

sgRNA-G-LRRC8A#1:
(SEQ ID NO: 54)
5′-GCTGCGTGTCCGCAAAGTAG;
and
sgRNA-G-LRRC8A#4:
(SEQ ID NO: 55)
5′-CCGGCACCAGTACAACTACG.

After adenoviral transduction, monoclonal HaCaT-LRRC8A^{-/-} cells were isolated from the heterogeneous cell pool by limiting dilution. The Cas9-mediated genomic deletion of LRRC8A was then confirmed by target site-specific PCR and subsequent Sanger sequencing. PCR amplification of the genomic region was performed using specific oligonucleotides (Seq_LRRC8A_24839 fw 5′-TGGTTTCCCAGCCAAGTG (SEQ ID NO:56); and Seq_LRRC8A_965 rv 5′-GCGGGAATTTGAACCAGAAG (SEQ ID NO:57)), dNTPs, Phusion Polymerase (Thermo Fischer) and genomic DNA as template. Genomic DNA was initially denatured at 98° C. for 30 sec followed by 30 amplification cycles including 10 sec denaturation at 98° C., 10 sec annealing at 65° C. and 30 sec elongation at 72° C. and a final elongation of 10 min at 72° C. PCR amplification products were separated on 2% agarose gels and visualized by Midori Green (Biozym) staining. PCR amplification products were purified using QuiQuick PCR Purifictaion Kit (Quiagen) and sequenced by Sanger sequencing using oligonucleotide Seq_LRRC8A_24839 fw (SEQ ID NO:56).

Measuring of Swelling-Induced VRAC Activity Using hsYFP

To measure VRAC activity in tissue culture cells the hsYFP gene expression cassette is delivered via adenoviral transduction. Therefore 0.25×10⁶ cells/well HaCaT cells were seeded in 6-well plates and incubated at 37° C. and 5% CO₂ for 1 day. Then, cells were transduced with adenovirus Ad-CMV-hsYFP (Sirion) at 300 MOI (multiplicities of infection) and incubated at 37° C. and 5% CO₂ for an additional day. 30,000 transduced cells were seeded in 96-well black-walled, clear bottom microplates (Costar) and cultivated for 1 day. Cells were washed three times with 70 μl isotonic incubation buffer (145 mM NaCl, 5 mM KCl, 1 mM MgCl₂, 2 mM CaCl₂, 10 mM glucose, 10 mM HEPES/NaOH, pH7.2, 329 mOsm) and incubated with 50 μl isotonic incubation buffer for 15 min at 37° C. Cellular hsYFP fluorescence (excitation at 485 nm, emission at 535 nm) was continuously recorded every 3.5 sec in an automated fluorescence plate reader. After baseline recording for 20 sec, cells were stimulated by addition of isotonic I⁻-solution (70 mM Nal, 5 mM NaCl, 140 mM mannitol, 5 mM KCl, 1 mM MgCl₂, 2 mM CaCl₂, 10 mM glucose, 10 mM HEPES/NaOH, pH7.2, 329 mOsm) or hypotonic I⁻-solution (70 mM Nal, 5 mM NaCl, 5 mM KCl, 1 mM MgCl₂, 2 mM CaCl₂, 10 mM glucose, 10 mM HEPES/NaOH, pH7.2, 189 mOsm) to establish an extracellular I⁻ concentration of 50 mM I⁻. Addition of hypotonic buffer resulted in a 30% decrease in osmolarity (final osmolarity of 229 mOsm). For experiments without extracellular calcium, 2 mM CaCl₂ were replaced by 2 mM MgCl₂.

HsYFP fluorescence (arbitrary units) was plotted over time and I⁻ influx rate was derived from the initial slope (ΔF fluorescence/Δtime) as a measure of swelling induced ion channel activity. VRAC activity was determined from at least four independent experiments performed in duplicates and were presented as the mean±standard deviation (SD). A two-sided, unpaired Student's t-test was performed for statistical analysis and significance was indicated as * p<0.05, **p<0.01 and ***p<0.001.

Measuring of Cell Volume Changes Using Calcein-AM

30,000 cells were seeded in 96-well black-walled, clear bottom microplates (Costar) and cultivated for 1 day. Prior to measurement, cells were loaded with 10 μM Calcein-AM (Fisher Scientific) in normal cultivation medium for 75 min at 37° C. For experiments investigating the influence of intracellular calcium, cells were additionally loaded with 20 μM BAPTA-AM. Cells were washed three times with 70 μl isotonic incubation buffer (145 mM NaCl, 5 mM KCl, 1 mM MgCl₂, 2 mM CaCl₂, 10 mM glucose, 10 mM HEPES/NaOH, pH7.2, 329 mOsm) and incubated in 50 μl isotonic incubation buffer at 37° C. Cellular calcein fluorescence (excitation at 485 nm, emission at 538 nm) was continuously recorded every 1.6 sec in an automated fluorescence plate reader. After baseline recording for 20 sec, cells were stimulated by addition of isotonic solution (20 mM NaCl, 250 mM mannitol, 5 mM KCl, 1 mM MgCl₂, 2 mM CaCl₂, 10 mM glucose, 10 mM HEPES/NaOH, pH7.2, 329 mOsm) or hypotonic solution (20 mM NaCl, 5 mM KCl, 1 mM MgCl₂, 2 mM CaCl₂, 10 mM glucose, 10 mM HEPES/NaOH, pH7.2, 79 mOsm). Addition of hypotonic buffer resulted in a 55% decrease in osmolarity (final osmolarity of 150 mOsm). Calcein fluorescence (arbitrary units) was plotted over time. Fluorescence (ΔF=Fₘₐₓ−F₀) before (F₀) and after (Fₘₐₓ) stimulation was normalized to baseline fluorescence (ΔF/F₀) and used as a relative measure for cell volume increase. Fluorescence quenching was derived from the slope (ΔF fluorescence/Δtime) after maximal fluorescence increase (Fₘₐₓ) and used as a measure of cell volume decrease. Cell volume changes were measured in two independent experiments carried out in triplicates and was presented as the mean±SD. A two-sided, unpaired Student's t-test was performed for statistical analysis and significance was indicated as * p<0.05, **p<0.01 and ***p<0.001.

Transcriptome Data Analysis of HaCaT Wildtype and LRRC8A Knock-Out Cells

Cell lysis and RNA isolation were performed using the NucleoSpin RNA Kit II from Macherey-Nagel. For library preparation, the TruSeq RNA Library Prep Kit v2 from Illumina was used starting with an input amount of 500 ng of total RNA. The prepared libraries were sequenced with a 2×150 bp read length using the HiSeq 3000/4000 SBS Kit and an Illumina Hiseq 4000 sequencer. The adapter trimmed, demultiplexed and quality filtered reads were aligned to the hg19 reference genome and transcriptome using Hisat2 (2.0.4)⁵³. The Hisat2 output files (SAM) were converted to the BAM format and were sorted and indexed using SAMtools (1.3.1)⁵⁴. The BAM files were evaluated by Cuffdiff2 (2.1.1), as previously described⁵⁵. The differential transcript abundance was calculated using Cuffdiff2. All samples were compared and evaluated in one calculation cycle, allowing the algorithm to estimate the FPKM values at the transcript level resolution and to control for variability across the replicate libraries⁵⁵. Cuffdiff2 was used because its normalization of read counts is equal to that of DESeq⁵⁵, which has been shown to be one of the most reliable normalization methods for RNA sequencing⁵⁶. Here, a scaling factor for each gene in a given sample was calculated as the median of the ratio of the read count of that gene over its geometric mean across all samples⁵⁷.

### Example 2: LRRC8A is Expressed in Cultured Human Keratinocytes and in Human Skin

First it was asked whether LRRC8A and its other LRRC family members (LRRC8B-E) are expressed in human skin cells. RT-PCR analysis was performed and showed that mRNA transcripts for all LRRC8 subunits are readily detectable in cells of the keratinocyte cell line HaCaT as well as in primary normal human epidermal keratinocytes (NHKs) (FIG. 1A). We also confirmed that mRNA transcripts are translated into LRRC8A protein by Western blot analysis using an LRRC8A-antibody. LRRC8A proteins could be detected in whole cell extracts of HaCaT cells as well as in NHKs (FIG. 1B).

It was next analyzed whether LRRC8A is also found in the context of normal human skin. LRRC8A-antibody staining was indeed observed in histological sections of human skin biopsies (FIG. 1C). Surprisingly, it was observed that LRRC8A is not uniformly expressed in all epidermal keratinocyte layers. LRRC8A was preferentially found in basal layers of the skin and decreased towards the outer layers (FIG. 1C). This observation suggests a differentiation-dependent LRRC8A expression, which led to the hypothesis that LRRC8A might have a not yet described role in terminal differentiation.

### Example 3: LRRC8A is Dynamically Regulated During Keratinocyte Differentiation

Since the gradual distribution of LRRC8A proteins along the epidermal keratinocyte layers (FIG. 1C) indicates that LRRC8A might also be involved in terminal differentiation, it was further explored how LRRC8A expression changes during the differentiation process of keratinocytes. For this purpose, differentiation was induced by cultivating HaCaT cells at post-mitotic cell densities. By Western blot analysis of whole cell extracts, it was observed that LRRC8A protein level first increased before it reached its maximum, and then declined again when terminal differentiation was achieved (FIG. 2A). This bell-shaped expression pattern was also confirmed by immunofluorescence analysis of cells of increasing differentiation status. Clearly, membrane localization of LRRC8A increased to its maximum and then decreased again with proceeding keratinocyte maturation (FIG. 2B). These findings show that LRRC8A is dynamically regulated during keratinocyte differentiation.

### Example 4: LRRC8A Expression is Changed in Inflammatory, Psoriasis-Like Keratinocytes and in Psoriasis Skin Lesions

After having found that LRRC8A is dynamically regulated during normal differentiation (FIG. 2A, B), it was next analysed whether LRRC8A might also be found in the context of aberrant differentiation processes such as occurring in the inflammatory skin disease psoriasis. In HaCaT cells differentiation was induced by post-confluent growth and in the presence of pro-inflammatory cytokines (IL-1β, IL-17A, TNF-α) to mimic the psoriatic conditions. Expression of LRRC8A as well as expression of the differentiation marker involucrin (IVL) was monitored by Western blot analysis (FIG. 3A, B). As expected, psoriasis-like HaCaT cells showed drastically delayed expression of IVL when compared to untreated HaCaT cells. Strikingly, also expression of LRRC8A was changed in psoriasis-like HaCaT cells (FIG. 3A, B). In contrast to the typical bell-shaped expression pattern that was observed during normal differentiation (FIG. 3A), LRRC8A proteins were detected much later and did not decrease at later stages of abnormal differentiation in psoriasis-like HaCaT cells (FIG. 3B).

Next it was asked whether LRRC8A is also changed in primary psoriatic keratinocytes and in skin lesions of psoriasis patients. Since it is difficult to obtain and analyze psoriatic skin biopsy samples, a search in publicly available whole transcriptome data from psoriatic skin lesions was initially conducted. Several studies were identified which gathered transcriptome data from primary keratinocytes treated with TNF-α and IL-17, which mimics a psoriatic, inflammatory skin cell, as well as studies from psoriasis patients^{5,7,41-43}. Evaluation of the data yielded that these studies focused on the importance of the most strongly affected genes in psoriasis. These are approx. 35 genes, which can be considered as key deregulated genes in psoriasis⁷, albeit, more than 200 genes^{5,41} and up to 2200 genes⁷ were described to be additionally changed. The function of many of these genes is not known and, therefore, scientific studies at present primarily address the function of the known genes. As a consequence, new potential key players and targets in psoriasis are often dismissed or overseen.

Nonetheless, the available transcriptome data were searched and it was asked whether LRRC8A is one of the so far overseen deregulated genes in psoriasis. Indeed, analysis of the supplementary data revealed that LRRC8A belongs to the list of differentially regulated genes in primary keratinocytes treated with TNF-α and IL-17⁵ (Table 3), which is in perfect accordance with the results from the psoriasis-like HaCaT cell model provided herein (FIG. 3B). It was also found that another LRRC8 family member, LRRCB, is among the list of genes that are deregulated in skin lesions of psoriasis patients, as judged from the transcriptome data⁴² (Table 3). Taken together, the experiments and data analysis provided herein strongly suggest that LRRC8A and other LRRC subunits are deregulated not only in inflammatory, psoriasis-like keratinocytes but also in psoriasis skin lesions and that LRRC8A might be a so far overseen player in psoriasis.

TABLE 3
The psoriatic model investigated, log2 change and fold
change of gene expression based on transcriptome data
are listed. Two genes of the LRRC8 gene family, LRRC8A
and LRRCB, are upregulated by approx. 1.5-2 in keratinocytes
treated with TNF-α and/or IL-17, which mimics
a psoriatic inflammatory skin cell, or in pathogenic
keratinocytes of psoriasis patients.

| | | | log2 | fold |
|---|---|---|---|---|
| Gene | Study | Psoriatic model | change | change |
| LRRC8A | Chiricozzi et al. | TNF-α | 0.510 | 1.42 |
| LRRC8B | Chiricozzi et al. | IL-17 + TNF-α | 0.562 | 1.47 |
| LRRC8B | Suarez-Farinas et al. | Patients | 1.100 | 2.14 |

Next it was investigated whether LRRC8A expression is changed in human skin of psoriasis patients (FIG. 3C). For this purpose, punch biopsies from diseased, lesional skin (a, d) and non-lesional skin (b, e) of two different psoriasis vulgaris patients (patient 1: a, b; patient 2: d, e) were compared with healthy donors (c, f). Healthy human skin showed strong LRRC8A staining (indicated as black color) in the epidermis and especially in the basal layer (c, f). In contrast non-lesional skin of psoriatic patients shows reduced staining for LRRC8A (b, e) while hardly any (d) or very weak (a) staining can be detected in lesional psoriatic skin. Thus, LRRCA protein levels are reduced in psoriatic epidermis.

### Example 5: Reduction of LRRC8A Activity by CRISPR/Cas9 Approach Influences VRAC Activity and RVD in HaCaT Keratinocytes

The deregulated expression of LRRC8A in psoriasis renders it a potentially attractive new target to modulate differentiation in psoriasis or other skin disorders such as atopic dermatitis, with the aim to attenuate the differentiation defects in the diseased skin cells. In order to prove that modulation of LRRC8A activity can be employed to manipulate the differentiation process, the differentiation process in the absence of LRRC8A activity was monitored.

For this purpose, a HaCaT-LRRC8A^{-/-} knock out cell line, which is devoid of functional LRRC8A, was created by employing the CRISPR-Cas genome editing technology. Two single-guide RNAs (sgRNA-G-LRRC8A #1: 5′-GCTGCGTGTCCGCAAAGTAG (SEQ ID NO:54); sgRNA-G-LRRC8A #4: 5′-CCGGCACCAGTACAACTACG (SEQ ID NO:55)) were designed to tether the Cas9 nuclease to defined regions in the genomic LRRC8A loci, which then leads to the defined deletion of approx. 300 bp of the LRRC8A coding sequence. The predicted LRRC8A gene deletion was confirmed by PCR using specific oligonucleotide primer pairs (Seq_LRRC8A_24839 fw 5′-TGGTTTCCCAGCCAAGTG (SEQ ID NO:56); Seq_LRRC8A_965 rv 5′-GCGGGAATTTGAACCAGAAG (SEQ ID NO:57)) and isolated genomic DNA (FIG. 4A). The constitutive LRRC8A gene disruption was confirmed by DNA sequencing of the genomic LRRC8A loci (data not shown) and by the absence of detectable LRRC8A protein in HaCaT-LRRC8A^{-/-} cells in Western blot analysis using whole cell lysates and anti-LRRC8A antibody (FIG. 4B).

It was then analyzed whether these HaCaT cells devoid of LRRC8A have a reduced VRAC activity and whether this influences cell volume regulation. Cell volume changes were determined by loading HaCaT cells with the fluorescent volume-sensitive dye calcein^{18,58,59}, whereas VRAC activity was determined by expressing the fluorescent halide-sensitive YFP (hsYFP), which allows to monitor the characteristic I⁻ influx of chloride channels including VRACs^{18,31,60-62}.

Strikingly, HaCaT-LRRC8A^{-/-} cells completely lacked VRAC activity upon hypotonic stimulation (FIG. 4C). Interestingly, cell swelling was only mildly affected whereas regulatory volume decrease (RVD) was drastically reduced in LRRC8A knock-out cells compared to HaCaT wildtype cells (FIG. 4D). These findings not only show for the first time that LRRC8A is clearly an essential component of VRACs also in HaCaT keratinocytes and mainly mediate RVD in HaCaTs, but it also proves that the approach to specifically reduce LRRC8A activity by reducing LRRC8A gene dosage via knocking out the LRRC8A gene is valid.

### Example 6: Modulation of LRRC8A Activity Influences Differentiation of Keratinocytes

After having established that the LRRC8A activity is almost completely diminished in HaCaT-LRRC8A−/− cells, this cell line was used to monitor the effect on keratinocyte differentiation in the absence of LRRC8A activity (FIG. 5A). The differentiation process was initiated by cultivating the HaCaT-LRRC8A−/− cells at post-mitotic cell densities and expression of the important differentiation markers involucrin (IVL) and keratin 10 (KRT10) was determined by Western blot analysis (FIG. 5A). Clearly, in HaCaT cells lacking LRRC8A activity, IVL occurred in much earlier stages of the differentiation process. Moreover, KRT10 protein level did not continuously increase but stayed at a low level throughout the entire differentiation (FIG. 5A). The manner in which IVL and KRT10 are changing are hallmarks of abnormal differentiation of keratinocytes showing that a decrease in LRRC8A activity leads to aberrant differentiation, which suggests that LRRC8A is required for proper differentiation.

To obtain a more comprehensive overview of other potentially deregulated genes, RNA-Seq analysis of HaCaT-LRRC8A^{-/-} cells was performed. In addition to IVL and KRT10 this transcriptomics approach revealed differential gene expression of additional crucial keratinocyte differentiation markers such as TGM1, KRT4 and KRT15 and markers for abnormal hyperproliferation KRT6 and KRT16. It was also found that several key genes, which were described to be deregulated in psoriasis^{5,7} are also deregulated in HaCaT cells devoid of LRRC8A (HaCaT-LRRC8A^{-/-} cells) (FIG. 5B, C).

In particular, Chiricozzi et al. had focused on a common set of 23 genes that are differentially expressed in keratinocytes treated with IL-17⁵. It was found by RNA sequencing in the present study that among those 23 genes, 6 genes were not expressed at all in HaCaT cells, 6 genes were not affected, and almost 50% of the genes (i.e. 11 of the 23 genes) were also deregulated in HaCaT-LRRC8A^{-/-} cells (FIG. 5B).

In the second study, Swindell et al. reported a group of 35 genes that are most strongly elevated in psoriasis lesions of patients⁷. Among those 35 genes, 14 genes were found by RNA sequencing that are not expressed at all in HaCaT cells, 10 genes were not affected, and approx. 30% of the genes (i.e. 11 of the 35 genes) were also deregulated in HaCaT-LRRC8A^{-/-} cells (FIG. 5C). This shows that not only single differentiation markers are deregulated, but that the lack of LRRC8A activity is instead linked to drastic gene expression changes including well-known genes that are linked to abnormal differentiation in psoriatic keratinocytes and in pathologic skin of psoriasis patients.

In summary, it was shown that a decrease in LRRC8A expression/activity leads to changed differentiation, i.e. it causes aberrant differentiation. Consequently, increasing LRRC8A activity will have beneficial effects on abnormal differentiation process, such as those found in psoriasis. Thus, differentiation can be affected by modulating LRRC8A activity, which can be used for the therapeutic treatment of differentiation defects, for example in psoriasis, or for cosmetically alleviating the effects of a skin condition, such as psoriasis, on the appearance of the skin of an affected individual.

### ADDITIONAL REFERENCES

1. El-Chami C, Haslam I S, Steward M C, O'Neill C A. Role of organic osmolytes in water homoeostasis in skin. Experimental dermatology. 2014; 23(8):534-537.
2. Warskulat U, Reinen A, Grether-Beck S, Krutmann J, Haussinger D. The osmolyte strategy of normal human keratinocytes in maintaining cell homeostasis. J Invest Dermatol. 2004; 123(3):516-521.
3. Boehncke W H, Schon M P. Psoriasis. Lancet. 2015; 386(9997):983-994.
4. Buerger C, Shirsath N, Lang V, et al. Inflammation dependent mTORC1 signaling interferes with the switch from keratinocyte proliferation to differentiation. PloS one. 2017, 12(7):e0180853.
5. Chiricozzi A, Guttman-Yassky E, Suarez-Farinas M, et al. Integrative responses to IL-17 and TNF-alpha in human keratinocytes account for key inflammatory pathogenic circuits in psoriasis. J Invest Dermatol. 2011, 131(3):677-687.
6. Guilloteau K, Paris I, Pedretti N, et al. Skin Inflammation Induced by the Synergistic Action of IL-17A, IL-22, Oncostatin M, IL-1{alpha}, and TNF-{alpha} Recapitulates Some Features of Psoriasis. J Immunol. 2010.
7. Swindell W R, Johnston A, Voorhees J J, Elder J T, Gudjonsson J E. Dissecting the psoriasis transcriptome: inflammatory- and cytokine-driven gene expression in lesions from 163 patients. BMC Genomics. 2013; 14:527.
8. Leigh I M, Naysaria H, Purkis P E, McKay I A, Bowden P E, Riddle P N. Keratins (K16 and K17) as markers of keratinocyte hyperproliferation in psoriasis in vivo and in vitro. Br J Dermatol. 1995, 133(4):501-511.
9. Nast A, Boehncke W H, Mrowietz U, et al. S3—Guidelines on the treatment of psoriasis vulgaris (English version). Update. J Dtsch Dermatol Ges. 2012; 10 Suppl 2:S1-95.
10. Mendonca C O, Burden A D. Current concepts in psoriasis and its treatment.

Pharmacol Ther. 2003, 99(2):133-147.
11. Jensen J M, Folster-Holst R, Baranowsky A, et al. Impaired sphingomyelinase activity and epidermal differentiation in atopic dermatitis. J Invest Dermatol. 2004; 122(6):1423-1431.
12. Harris V R, Cooper A J. Atopic dermatitis: the new frontier. Med J Aust. 2017; 207(8):351-356.
13. Cotter D G, Schairer D, Eichenfield L. Emerging Therapies for Atopic Dermatitis: Jak Inhibitors. Journal of the American Academy of Dermatology. 2017.
14. Wu J J, Lynde C W, Kleyn C E, et al. Identification of key research needs for topical therapy treatment of psoriasis—a consensus paper by the International Psoriasis Council. J Eur Acad Dermatol Venereol. 2016; 30(7):1115-1119.
15. Stauber T. The volume-regulated anion channel is formed by LRRC8 heteromers—molecular identification and roles in membrane transport and physiology. Biol Chem. 2015, 396(9-10):975-990.
16. Abascal F, Zardoya R. LRRC8 proteins share a common ancestor with pannexins, and may form hexameric channels involved in cell-cell communication. Bioessays. 2012; 34(7):551-560.
17. Syeda R, Qiu Z, Dubin A E, et al. LRRC8 Proteins Form Volume-Regulated Anion Channels that Sense Ionic Strength. Cell. 2016, 164(3):499-511.
18. Voss F K, Ullrich F, Munch J, et al. Identification of LRRC8 heteromers as an essential component of the volume-regulated anion channel VRAC. Science. 2014, 344(6184):634-638.
19. Kumar L, Chou J, Yee C S, et al. Leucine-rich repeat containing 8A (LRRC8A) is essential for T lymphocyte development and function. J Exp Med. 2014, 211(5):929-942.
20. Zhang Y, Xie L, Gunasekar S K, et al. SWELL1 is a regulator of adipocyte size, insulin signalling and glucose homeostasis. Nat Cell Biol. 2017; 19(5):504-517.
21. Choi H, Ettinger N, Rohrbough J, Dikalova A, Nguyen H N, Lamb F S. LRRC8A channels support TNFalpha-induced superoxide production by Nox1 which is required for receptor endocytosis. Free Radic Biol Med. 2016; 101:413-423.
22. Wang R, Lu Y, Gunasekar S, et al. The volume-regulated anion channel (LRRC8) in nodose neurons is sensitive to acidic pH. JCI Insight. 2017; 2(5):e90632.
23. Wang H, La Russa M, Qi L S. CRISPR/Cas9 in Genome Editing and Beyond. Annu Rev Biochem. 2016; 85:227-264.
24. Tak Y E, Kleinstiver B P, Nunez J K, et al. Inducible and multiplex gene regulation using CRISPR-Cpf1-based transcription factors. Nat Methods. 2017; 14(12):1163-1166.
25. Konermann S, Brigham M D, Trevino A E, et al. Genome-scale transcriptional activation by an engineered CRISPR-Cas9 complex. Nature. 2015, 517(7536):583-588.
26. Silva G, Poirot L, Galetto R, et al. Meganucleases and other tools for targeted genome engineering: perspectives and challenges for gene therapy. Curr Gene Ther. 2011; 11(1):11-27.
27. Miller J C, Tan S, Qiao G, et al. A TALE nuclease architecture for efficient genome editing. Nature biotechnology. 2011, 29(2):143-148.
28. Klug A. The discovery of zinc fingers and their applications in gene regulation and genome manipulation. Annual review of biochemistry. 2010; 79:213-231.
29. Guo M, Pegoraro A F, Mao A, et al. Cell volume change through water efflux impacts cell stiffness and stem cell fate. Proceedings of the National Academy of Sciences of the United States of America. 2017, 114(41):E8618-E8627.
30. Lutter D, Ullrich F, Lueck J C, Kempa S, Jentsch T J. Selective transport of neurotransmitters and modulators by distinct volume-regulated LRRC8 anion channels. Journal of cell science. 2017; 130(6):1122-1133.
31. Qiu Z, Dubin A E, Mathur J, et al. SWELL1, a plasma membrane protein, is an essential component of volume-regulated anion channel. Cell. 2014; 157(2):447-458.
32. Hoffmann E K, Lambert I H, Pedersen S F. Physiology of cell volume regulation in vertebrates. Physiol Rev. 2009; 89(1):193-277.
33. Jentsch T J. VRACs and other ion channels and transporters in the regulation of cell volume and beyond. Nat Rev Mol Cell Biol. 2016; 17(5):293-307.
34. Okada Y, Sato K, Numata T. Pathophysiology and puzzles of the volume-sensitive outwardly rectifying anion channel. The Journal of physiology. 2009; 587(Pt 10):2141-2149.
35. Dubois J M, Rouzaire-Dubois B. Roles of cell volume in molecular and cellular biology. Prog Biophys Mol Biol. 2012; 108(3):93-97.
36. Pedersen S F, Hoffmann E K, Novak I. Cell volume regulation in epithelial physiology and cancer. Front Physiol. 2013; 4:233.
37. Barrandon Y, Green H. Cell size as a determinant of the clone-forming ability of human keratinocytes. Proceedings of the National Academy of Sciences of the United States of America. 1985, 82(16):5390-5394.
38. Dascalu A, Matithyou A, Oron Y, Korenstein R. A hyperosmotic stimulus elevates intracellular calcium and inhibits proliferation of a human keratinocyte cell line. J Invest Dermatol. 2000, 115(4):714-718.
39. Gonczi M, Szentandrassy N, Fulop L, et al. Hypotonic stress influence the membrane potential and alter the proliferation of keratinocytes in vitro. Experimental dermatology. 2007; 16(4):302-310.
40. Kippenberger S, Loitsch S, Guschel M, Muller J, Kaufmann R, Bernd A. Hypotonic stress induces E-cadherin expression in cultured human keratinocytes. FEBS Lett. 2005; 579(1):207-214.
41. Gudjonsson J E, Ding J, Li X, et al. Global gene expression analysis reveals evidence for decreased lipid biosynthesis and increased innate immunity in uninvolved psoriatic skin. J Invest Dermatol. 2009, 129(12):2795-2804.
42. Suarez-Farinas M, Lowes M A, Zaba L C, Krueger J G. Evaluation of the psoriasis transcriptome across different studies by gene set enrichment analysis (GSEA). PloS one. 2010; 5(4):e10247.
43. Yao Y, Richman L, Morehouse C, et al. Type I interferon: potential therapeutic target for psoriasis? PloS one. 2008; 3(7):e2737.
44. Lowes M A, Russell C B, Martin D A, Towne J E, Krueger J G. The IL-23/T17 pathogenic axis in psoriasis is amplified by keratinocyte responses. Trends Immunol. 2013, 34(4):174-181.
45. Proksch E, Folster-Holst R, Jensen J M. Skin barrier function, epidermal proliferation and differentiation in eczema. J Dermatol Sci. 2006, 43(3):159-169.
46. Thomsen S F. Atopic dermatitis: natural history, diagnosis, and treatment. ISRN Allergy. 2014; 2014:354250.
47. Soboleva A G, Mezentsev A, Zolotorenko A, Bruskin S, Pirusian E. Three-dimensional skin models of psoriasis. Cells Tissues Organs. 2014; 199(5-6):301-310.
48. van den Bogaard E H, Tjabringa G S, Joosten I, et al. Crosstalk between keratinocytes and T cells in a 3D microenvironment: a model to study inflammatory skin diseases. J Invest Dermatol. 2014, 134(3):719-727.
49. Lane M E. Skin penetration enhancers. Int J Pharm. 2013; 447(1-2):12-21.
50. Zoller N N, Kippenberger S, Thaci D, et al. Evaluation of beneficial and adverse effects of glucocorticoids on a newly developed full-thickness skin model. Toxicol In Vitro. 2008, 22(3):747-759.
51. Malisiewicz B, Boehncke S, Lang V, Boehncke W H, Buerger C. Epidermal insulin resistance as a therapeutic target in acanthosis nigricans? Acta Derm Venereol. 2014, 94(5):607-608.
52. Buerger C, DeVries B, Stambolic V. Localization of Rheb to the endomembrane is critical for its signaling function. Biochemical and biophysical research communications. 2006, 344(3):869-880.
53. Kim D, Langmead B, Salzberg S L. HISAT: a fast spliced aligner with low memory requirements. Nat Methods. 2015, 12(4):357-360.
54. Li H, Handsaker B, Wysoker A, et al. The Sequence Alignment/Map format and SAMtools. Bioinformatics. 2009; 25(16):2078-2079.
55. Trapnell C, Hendrickson D G, Sauvageau M, Goff L, Rinn J L, Pachter L. Differential analysis of gene regulation at transcript resolution with RNA-seq. Nat Biotechnol. 2013, 31(1):46-53.
56. Dillies M A, Rau A, Aubert J, et al. A comprehensive evaluation of normalization methods for Illumina high-throughput RNA sequencing data analysis. Brief Bioinform. 2013; 14(6):671-683.
57. Anders S, Huber W. Differential expression analysis for sequence count data. Genome Biol. 2010; 11(10):R106.
58. Hamann S, Kiilgaard J F, Litman T, Avarez-Leefmans F J, Winther B R, Zeuthen T. Measurement of cell volume changes by fluorescence self-quenching. J Fluoresc. 2002, 12(2):139-145.
59. Davidson A F, Higgins A Z. Detection of volume changes in calcein-stained cells using confocal microscopy. J Fluoresc. 2013; 23(3):393-398.
60. Ertongur-Fauth T, Hochheimer A, Buescher J M, Rapprich S, Krohn M. A novel TMEM16A splice variant lacking the dimerization domain contributes to calcium-activated chloride secretion in human sweat gland epithelial cells. Experimental dermatology. 2014, 23(11):825-831.
61. Galietta L V, Jayaraman S, Verkman A S. Cell-based assay for high-throughput quantitative screening of CFTR chloride transport agonists. American journal of physiology. 2001, 281(5):C1734-1742.
62. Galietta L J, Haggie P M, Verkman A S. Green fluorescent protein-based halide indicators with improved chloride and iodide affinities. FEBS Lett. 2001, 499(3):220-224.

1. The leucine-rich repeat-containing protein 8A (LRRC8A), and/or an activator of LRRC8A, for use in the treatment and/or prevention of a skin condition associated with an altered differentiation of keratinocytes.

2. The LRRC8A and/or the activator for use according to claim 1, wherein the skin condition associated with an altered differentiation of keratinocytes is a condition characterised by enhanced epidermal proliferation.

3. The LRRC8A and/or the activator for use according to claim 1, wherein the skin condition associated with an altered differentiation of keratinocytes is psoriasis or dermatitis, preferably atopic dermatitis.

4. The activator of LRRC8A for use according to claim 1, wherein the activator is
(i) a vector encoding, in expressible form, LRRC8A; or
(ii) a regulator of gene expression that up-regulates the expression of endogenously present LRRC8A.

5. The activator of LRRC8A for use according to claim 4 (ii), wherein the regulator of gene expression that up-regulates the expression of endogenously present LRRC8A is selected from (i) CRISPR-Cas9-based regulators; (ii) CRISPR-Cpf1-based regulators; (iii) programmable sequence-specific genome editing nucleases selected from zinc-finger nucleases (ZNFs) and transcriptional activator-like effector nucleases (TALENs); (iv) meganucleases; (v) small molecules; (vi) antibodies or antibody mimetics; (vii) aptamers; and (viii) inhibitory nucleic acid molecules selected from siRNA, shRNA, miRNA, ribozymes and antisense nucleic acid molecules.

6. A method of identifying a compound capable of altering the differentiation of keratinocytes, the method comprising the steps of
(a) contacting keratinocytes with a test compound and determining the amount of LRRC8A protein or LRRC8A transcript in said keratinocytes; and
(b) comparing the amount of LRRC8A protein or LRRC8A transcript determined in step (a) with the amount of LRRC8A protein or LRRC8A transcript in a control not contacted with said test compound,
wherein a change in the amount of LRRC8A protein or LRRC8A transcript after contacting the keratinocytes with the test compound indicates that the test compound is capable of altering the differentiation of keratinocytes.

7. A method of identifying a compound capable of altering the differentiation of keratinocytes, the method comprising the steps of
(a) contacting keratinocytes with a test compound and determining the activity of (a) VRAC(s) comprising LRRC8A in said keratinocytes; and
(b) comparing the activity determined in step (a) with the activity in a control not contacted with said test compound,
wherein a change in the activity of (a) VRAC(s) comprising LRRC8A after contacting the keratinocytes with the test compound indicates that the test compound is capable of altering the differentiation of keratinocytes.

8. The method of claim 6 or 7, further comprising determining the expression level of at least one marker selected from keratin 1 (KRT1), keratin 10 (KRT10), involucrin (IVL), filaggrin (FLG), loricrin (LOR), keratin 4 (KRT4), keratin 15 (KRT15), transglutaminase 1 (TGM1), S100 calcium binding protein A7 (S100A7), S100 calcium binding protein A8 (S100A8), S100 calcium binding protein A9 (S100A9), C-X-C motif chemokine ligand 1 (CXCL1), C-X-C motif chemokine ligand 8 (CXCL8), small proline rich protein 2C (SPRR2C), small proline rich protein 2D (SPRR2D), serpin family B member 3 (SERPINB3), serpin family B member 4 (SERPINB4), peptidase inhibitor 3 (PI3), lipocalin 2 (LCN2), keratin 6A (KRT6A), keratin 16 (KRT16), beta-defensin 1 (DEFB1) and marker of proliferation Ki-67 (MK167)

9. The method of claim 6, wherein an increase in the amount of LRRC8A protein or LRRC8A transcript after contacting the keratinocytes with the test compound and/or an increase in the activity of (a) VRAC(s) comprising LRRC8A after contacting the keratinocytes with the test compound indicates that the test compound is a compound suitable for use in the treatment and/or prevention of a skin condition associated with an altered differentiation of keratinocytes.

10. The method of claim 9, wherein the skin condition associated with an altered differentiation of keratinocytes is psoriasis or dermatitis, preferably atopic dermatitis.

11. An inhibitor of the leucine-rich repeat-containing protein 8A (LRRC8A) for use in the treatment and/or prevention of a skin condition selected from skin injury and impaired wound healing.

12. The inhibitor for use according to claim 11, wherein
(i) the inhibitor decreases the expression of LRRC8A; and/or
(ii) the inhibitor decreases the activity of volume-regulated anion channels (VRACs) comprising LRRC8A.

13. The LRRC8A and/or the activator for use according to claim 1, wherein the LRRC8A and/or the activator is comprised in a pharmaceutical composition.

14. The method of claim 6, wherein a decrease in the amount of LRRC8A protein or LRRC8A transcript after contacting the keratinocytes with the test compound and/or a decrease in the activity of (a) VRAC(s) comprising LRRC8A after contacting the keratinocytes with the test compound indicates that the test compound is a compound suitable for use in the treatment and/or prevention of a skin condition selected from skin injury and impaired wound healing.

15. A cosmetic method for treating the skin of an individual, the method comprising topically administering an effective amount of
(i) leucine-rich repeat-containing protein 8A (LRRC8A);
(ii) an activator of LRRC8A;
(iii) LRRC8A and an activator of LRRC8A; or
(iv) an inhibitor of LRRC8A.

16. The inhibitor for use according to claim 11, wherein the inhibitor is comprised in a pharmaceutical composition.
